SEQUENCE LISTING (64 sequences)

SEQ ID NO: 1 - DNA, 2301 bp, Homo sapiens
atggcggcgctgagcggtggcggtggtggcggcgcggagccgggccaggctctgttcaacggggacatggagcccgaggccggcgccggcgccggcgccgcggcctcttcggctgcggaccctgccattccggaggaggtgtggaatatcaaacaaatgattaagttgacacaggaacatatagaggccctattggacaaatttggtggggagcataatccaccatcaatatatctggaggcctatgaagaatacaccagcaagctagatgcactccaacaaagagaacaacagttattggaatctctggggaacggaactgatttttctgtttctagctctgcatcaatggataccgttacatcttcttcctcttctagcctttcagtgctaccttcatctctttcagtttttcaaaatcccacagatgtggcacggagcaaccccaagtcaccacaaaaacctatcgttagagtcttcctgcccaacaaacagaggacagtggtacctgcaaggtgtggagttacagtccgagacagtctaaagaaagcactgatgatgagaggtctaatcccagagtgctgtgctgtttacagaattcaggatggagagaagaaaccaattggttgggacactgatatttcctggcttactggagaagaattgcatgtggaagtgttggagaatgttccacttacaacacacaactttgtacgaaaaacgtttttcaccttagcattttgtgacttttgtcgaaagctgcttttccagggtttccgctgtcaaacatgtggttataaatttcaccagcgttgtagtacagaagttccactgatgtgtgttaattatgaccaacttgatttgctgtttgtctccaagttctttgaacaccacccaataccacaggaagaggcgtccttagcagagactgccctaacatctggatcatccccttccgcacccgcctcggactctattgggccccaaattctcaccagtccgtctccttcaaaatccattccaattccacagcccttccgaccagcagatgaagatcatcgaaatcaatttgggcaacgagaccgatcctcatcagctcccaatgtgcatataaacacaatagaacctgtcaatattgatgacttgattagagaccaaggatttcgtggtgatggaggatcaaccacaggtttgtctgctaccccccctgcctcattacctggctcactaactaacgtgaaagccttacagaaatctccaggacctcagcgagaaaggaagtcatcttcatcctcagaagacaggaatcgaatgaaaacacttggtagacgggactcgagtgatgattgggagattcctgatgggcagattacagtgggacaaagaattggatctggatcatttggaacagtctacaagggaaagtggcatggtgatgtggcagtgaaaatgttgaatgtgacagcacctacacctcagcagttacaagccttcaaaaatgaagtaggagtactcaggaaaacacgacatgtgaatatcctactcttcatgggctattccacaaagccacaactggctattgttacccagtggtgtgagggctccagcttgtatcaccatctccatatcattgagaccaaatttgagatgatcaaacttatagatattgcacgacagactgcacagggcatggattacttacacgccaagtcaatcatccacagagacctcaagagtaataatatatttcttcatgaagacctcacagtaaaaataggtgattttggtctagctacagtgaaatctcgatggagtgggtcccatcagtttgaacagttgtctggatccattttgtggatggcaccagaagtcatcagaatgcaagataaaaatccatacagctttcagtcagatgtatatgcatttggaattgttctgtatgaattgatgactggacagttaccttattcaaacatcaacaacagggaccagataatttttatggtgggacgaggatacctgtctccagatctcagtaaggtacggagtaactgtccaaaagccatgaagagattaatggcagagtgcctcaaaaagaaaagagatgagagaccactctttccccaaattctcgcctctattgagctgctggcccgctcattgccaaaaattcaccgcagtgcatcagaaccctccttgaatcgggctggtttccaaacagaggattttagtctatatgcttgtgcttctccaaaaacacccatccaggcagggggatatggtgcgtttcctgtccactga

SEQ ID NO: 2 - protein, 766 aa, Homo sapiens [sequence omitted]

SEQ ID NO: 3 - DNA, 1212 bp, Homo sapiens
atgacagccatcatcaaagagatcgttagcagaaacaaaaggagatatcaagaggatggattcgacttagacttgacctatatttatccaaacattattgctatgggatttcctgcagaaagacttgaaggcgtatacaggaacaatattgatgatgtagtaaggtttttggattcaaagcataaaaaccattacaagatatacaatctttgtgctgaaagacattatgacaccgccaaatttaattgcagagttgcacaatatccttttgaagaccataacccaccacagctagaacttatcaaacccttttgtgaagatcttgaccaatggctaagtgaagatgacaatcatgttgcagcaattcactgtaaagctggaaagggacgaactggtgtaatgatatgtgcatatttattacatcggggcaaatttttaaaggcacaagaggccctagatttctatggggaagtaaggaccagagacaaaaagggagtaactattcccagtcagaggcgctatgtgtattattatagctacctgttaaagaatcatctggattatagaccagtggcactgttgtttcacaagatgatgtttgaaactattccaatgttcagtggcggaacttgcaatcctcagtttgtggtctgccagctaaaggtgaagatatattcctccaattcaggacccacacgacgggaagacaagttcatgtactttgagttccctcagccgttacctgtgtgtggtgatatcaaagtagagttcttccacaaacagaacaagatgctaaaaaaggacaaaatgtttcacttttgggtaaatacattcttcataccaggaccagaggaaacctcagaaaaagtagaaaatggaagtctatgtgatcaagaaatcgatagcatttgcagtatagagcgtgcagataatgacaaggaatatctagtacttactttaacaaaaaatgatcttgacaaagcaaataaagacaaagccaaccgatacttttctccaaattttaaggtgaagctgtacttcacaaaaacagtagaggagccgtcaaatccagaggctagcagttcaacttctgtaacaccagatgttagtgacaatgaacctgatcattatagatattctgacaccactgactctgatccagagaatgaaccttttgatgaagatcagcatacacaaattacaaaagtctga

SEQ ID NO: 4 - protein, 403 aa, Homo sapiens [sequence omitted]

SEQ ID NO: 5 - DNA, 1425 bp, Homo sapiens
atgaacaagctgagtggaggcggcgggcgcaggactcgggtggaagggggccagcttgggggcgaggagtggacccgccacgggagctttgtcaataagcccacgcggggctggctgcatcccaacgacaaagtcatgggacccggggtttcctacttggttcggtacatgggttgtgtggaggtcctccagtcaatgcgtgccctggacttcaacacccggactcaggtcaccagggaggccatcagtctggtgtgtgaggctgtgccgggtgctaagggggcgacaaggaggagaaagccctgtagccgcccgctcagctctatcctggggaggagtaacctgaaatttgctggaatgccaatcactctcaccgtctccaccagcagcctcaacctcatggccgcagactgcaaacagatcatcgccaaccaccacatgcaatctatctcatttgcatccggcggggatccggacacagccgagtatgtcgcctatgttgccaaagaccctgtgaatcagagagcctgccacattctggagtgtcccgaagggcttgcccaggatgtcatcagcaccattggccaggccttcgagttgcgcttcaaacaatacctcaggaacccacccaaactggtcacccctcatgacaggatggctggctttgatggctcagcatgggatgaggaggaggaagagccacctgaccatcagtactataatgacttcccggggaaggaaccccccttggggggggtggtagacatgaggcttcgggaaggagccgctccaggggctgctcgacccactgcacccaatgcccagacccccagccacttgggagctacattgcctgtaggacagcctgttgggggagatccagaagtccgcaaacagatgccacctccaccaccctgtccagcaggcagagagctttttgatgatccctcctatgtcaacgtccagaacctagacaaggcccggcaagcagtgggtggtgctgggccccccaatcctgctatcaatggcagtgcaccccgggacctgtttgacatgaagcccttcgaagatgctcttcgcgtgcctccacctccccagtcggtgtccatggctgagcagctccgaggggagccctggttccatgggaagctgagccggcgggaggctgaggcactgctgcagctcaatggggacttcctggtacgggagagcacgaccacacctggccagtatgtgctcactggcttgcagagtgggcagcctaagcatttgctactggtggaccctgagggtgtggttcggactaaggatcaccgctttgaaagtgtcagtcaccttatcagctaccacatggacaatcacttgcccatcatctctgcgggcagcgaactgtgtctacagcaacctgtggagcggaaactgtga

SEQ ID NO: 6 - protein, 474 aa, Homo sapiens
MNKLSGGGGRRTRVEGGQLGGEEWTRHGSFVNKPTRGWLHPNDKVMGPGVSYLVRYMGCVEVLQSMRALDFNTRTQVTREAISLVCEAVPGAKGATRRRKPCSRPLSSILGRSNLKFAGMPITLTVSTSSLNLMAADCKQIIANHHMQSISFASGGDPDTAEYVAYVAKDPVNQRACHILECPEGLAQDVISTIGQAFELRFKQYLRNPPKLVTPHDRMAGFDGSAWDEEEEEPPDHQYYNDFPGKEPPLGGVVDMRLREGAAPGAARPTAPNAQTPSHLGATLPVGQPVGGDPEVRKQMPPPPPCPAGRELFDDPSYVNVQNLDKARQAVGGAGPPNPAINGSAPRDLFDMKPFEDALRVPPPPQSVSMAEQLRGEPWFHGKLSRREAEALLQLNGDFLVRESTTTPGQYVLTGLQSGQPKHLLLVDPEGVVRTKDHRFESVSHLISYHMDNHLPIISAGSELCLQQPVERKL

SEQ ID NO: 7 - DNA, 639 bp, Homo sapiens
atgaactccttctccacaagcgccttcggtccagttgccttctccctggggctgctcctggtgttgcctgctgccttccctgccccagtacccccaggagaagattccaaagatgtagccgccccacacagacagccactcacctcttcagaacgaattgacaaacaaattcggtacatcctcgacggcatctcagccctgagaaaggagacatgtaacaagagtaacatgtgtgaaagcagcaaagaggcactggcagaaaacaacctgaaccttccaaagatggctgaaaaagatggatgcttccaatctggattcaatgaggagacttgcctggtgaaaatcatcactggtcttttggagtttgaggtatacctagagtacctccagaacagatttgagagtagtgaggaacaagccagagctgtgcagatgagtacaaaagtcctgatccagttcctgcagaaaaaggcaaagaatctagatgcaataaccacccctgacccaaccacaaatgccagcctgctgacgaagctgcaggcacagaaccagtggctgcaggacatgacaactcatctcattctgcgcagctttaaggagttcctgcagtccagcctgagggctcttcggcaaatgtag

SEQ ID NO: 8 - protein, 212 aa, Homo sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 9 - DNA, 1071 bp, Homo sapiens
atgtccattcctttgcctcttttgcagatatacacttcagataactacaccgaggaaatgggctcaggggactatgactccatgaaggaaccctgtttccgtgaagaaaatgctaatttcaataaaatcttcctgcccaccatctactccatcatcttcttaactggcattgtgggcaatggattggtcatcctggtcatgggttaccagaagaaactgagaagcatgacggacaagtacaggctgcacctgtcagtggccgacctcctctttgtcatcacgcttcccttctgggcagttgatgccgtggcaaactggtactttgggaacttcctatgcaaggcagtccatgtcatctacacagtcaacctctacagcagtgtcctcatcctggccttcatcagtctggaccgctacctggccatcgtccacgccaccaacagtcagaggccaaggaagctgttggctgaaaaggtggtctatgttggcgtctggatccctgccctcctgctgactattcccgacttcatctttgccaacgtcagtgaggcagatgacagatatatctgtgaccgcttctaccccaatgacttgtgggtggttgtgttccagtttcagcacatcatggttggccttatcctgcctggtattgtcatcctgtcctgctattgcattatcatctccaagctgtcacactccaagggccaccagaagcgcaaggccctcaagaccacagtcatcctcatcctggctttcttcgcctgttggctgccttactacattgggatcagcatcgactccttcatcctcctggaaatcatcaagcaagggtgtgagtttgagaacactgtgcacaagtggatttccatcaccgaggccctagctttcttccactgttgtctgaaccccatcctctatgctttccttggagccaaatttaaaacctctgcccagcacgcactcacctctgtgagcagagggtccagcctcaagatcctctccaaaggaaagcgaggtggacattcatctgtttccactgagtctgagtcttcaagttttcactccagctaa

SEQ ID NO: 10 - protein, 356 aa, Homo sapiens
MSIPLPLLQIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 11 - DNA, 5013 bp, Homo sapiens
atgagcgcccggaccgcccccaggccgcaggtgctcctgctgccgctcctgctggtgctcctggcggcggcgcccgcagccagcaagggttgtgtctgtaaagacaaaggccagtgcttctgtgacggggccaaaggggagaagggggagaagggctttcctggaccccccggttctcctggccagaaaggattcacaggtcctgaaggcttgcctggaccgcagggacccaagggctttccaggacttccaggactcacgggttccaaaggtgtaaggggaataagtggattgccaggattttctggttctcctggacttccaggcaccccaggcaataccgggccttacggacttgtcggtgtaccaggatgcagtggttctaagggtgagcaggggtttccaggactcccagggacactgggctacccagggatcccgggtgctgctggtttgaaaggacaaaagggtgctcctgctaaagaagaagatatagaacttgatgcaaaaggcgaccccgggttgccaggggctccaggaccccagggtttgccaggccctccaggttttcctgggcctgttggcccacctggtcctccgggattctttggctttccaggagccatgggacctagaggacctaagggtcacatgggtgaaagagtgataggacataaaggagagcggggtgtgaaagggttaacaggacccccgggaccaccaggaacagttattgtgaccctaactggcccagataacagaacggacctcaagggggaaaagggagacaagggagcaatgggcgagcctggacctcctggaccctcaggactgcctggagaatcatatggatctgaaaagggtgctcctggagaccctggcctgcagggaaaacccggaaaagatggtgttcctggcttccctggaagtgagggagtcaagggcaacaggggtttccctgggttaatgggtgaagatggcattaagggacagaaaggggacattggccctccaggatttcgtggtccaacagaatattatgacacataccaggaaaagggagatgaaggcactccaggcccaccagggcccagaggagctcgtggcccacaaggtcccagtggtccccccggagttcctggaagtcctggatcatcaaggcctggcctcagaggagcccctggatggccaggcctgaaaggaagtaaaggggaacgaggccgcccaggaaaggatgccatggggactcctgggtccccaggttgtgctggttcaccaggtcttccaggatcaccgggacctccaggaccgccaggtgacatcgtttttcgcaagggtccacctggagatcacggactgccaggctatctagggtctccaggaatcccaggagttgatgggcccaaaggagaaccaggcctcctgtgtacacagtgcccttatatcccagggcctcccggtctcccaggattgccagggttacatggtgtaaaaggaatcccaggaagacaaggcgcagctggcttgaaaggaagcccagggtccccaggaaatacaggtcttccaggatttccaggtttcccaggtgcccagggtgacccaggacttaaaggagaaaaaggtgaaacacttcagcctgaggggcaagtgggtgtcccaggtgacccggggctcagaggccaacctgggagaaagggcttggatggaattcctggaactccgggagtgaaaggattaccaggacctaaaggcgaactggctctgagtggtgagaaaggggaccaaggtcctccaggggatcctggctcccctgggtccccaggacctgcaggaccagctggaccacctggctacggaccccaaggagaacctggtctccagggcacgcaaggagttcctggagcccccggaccacccggagaagccggccctaggggagagctcagtgtttcaacaccagttccaggcccaccaggacctccagggccccctggccatcctggcccccaaggtccacctggtatccctggatccctggggaaatgtggagatcctggtcttccagggcctgatggtgaaccaggaattccaggaattggatttcctgggcctcctggacctaagggagaccaaggttttccaggtacaaaaggatcactgggttgtcctggaaaaatgggagagcctgggttacctggaaagccaggcctcccaggagccaagggagaaccagcagtagccatgcctggaggaccaggaacaccaggttttccaggagaaagaggcaattctggggaacatggagaaattggactccctggacttccaggtctccctggaactccaggaaatgaagggcttgatggaccacgaggagatccagggcagcctggaccacctggagaacaaggacccccaggaaggtgcatagagggtcccaggggagcccaaggacttccaggcttaaatggattgaaagggcaacaaggcagaagaggtaaaacggggccaaagggagacccaggaattccaggcttggatagatcaggatttcctggagaaactggatcaccaggaattccaggtcatcaaggtgaaatgggaccactgggtcaaagaggatatccaggaaatccgggaattttagggccaccaggtgaagatggagtgattgggatgatgggctttcctggagccattggccctccagggccccctgggaacccaggcacaccagggcagagggggagccctggaattccaggagtaaagggccagagaggaaccccaggagccaagggggaacaaggagataaaggaaatcccgggccttcagagatatcccacgtaataggggacaaaggagaaccaggtctcaaaggattcgcaggaaatccaggtgagaaaggaaacagaggcgttccagggatgccaggtttaaagggcctcaaaggactacccggaccagcaggaccaccaggccccagaggagatttgggcagcactgggaatcctggagaaccaggactgcgtggtataccaggaagcatggggaacatgggcatgccaggttctaaaggaaaaaggggaactttgggattcccaggtcgagcaggaagaccaggcctcccaggtattcatggtctccagggagataagggagagccaggttattcagaaggtacaaggccaggaccaccgggaccaacgggggatccaggactgccgggtgatatgggaaagaaaggagaaatggggcaacctggcccacctggacatttggggcctgctggacctgagggagcccctggaagtcctggaagtcctggcctcccaggaaagccaggtcctcatggtgatttgggttttaaaggaatcaaaggcctcctgggccctccaggaatcagaggccctccaggtcttccaggatttccaggatctcctggaccaatgggtataagaggtgaccaaggacgtgatggaattcctggtccagccggagaaaagggagaaacgggtttattgagggcccctccaggcccaagagggaaccctggtgctcaaggagccaaaggagacaggggagccccaggttttcctggcctcccgggcagaaaaggggccatgggagatgctggacctcgaggacccacaggcatagaaggattcccagggccaccaggtctgcccggtgcaattatccctggccagacaggaaatcgtggtccaccaggctcaagaggaagcccaggtgcgcctggtccccctggacctccagggagtcatgtaataggcataaaaggagacaaagggtctatgggccaccctggcccaaaaggtccacctggaactgcaggagacatgggaccaccaggtcgtctgggagcaccaggtactccaggtcttccaggacccagaggtgatcctggattccaggggtttccaggcgtgaaaggagaaaagggtaatcctggatttctaggatccattggacctccaggaccaattgggccaaaaggaccacctggtgtacgtggagaccctggcacacttaagattatctcccttccaggaagcccagggccacctggcacacctggagaaccagggatgcagggagaacctgggccaccagggccacctggaaacctaggaccctgtgggccaagaggtaagccaggcaaggatggaaaaccaggaactcctggaccagctggagaaaaaggcaacaaaggttctaaaggagagccaggaccagctggatcagatggattgccaggtttgaaaggaaaacgtggagacagtggatcacctgcaacctggacaacgagaggctttgtcttcacccgacacagtcaaaccacagcaattccttcatgtccagaggggacagtgccactctacagtgggttttcttttctttttgtacaaggaaatcaacgagcccacggacaagaccttggaactcttggcagctgcctgcagcgatttaccacaatgccattcttattctgcaatgtcaatgatgtatgtaattttgcatctcgaaatgattattcatactggctgtcaacaccagctctgatgccaatgaacatggctcccattactggcagagcccttgagccttatataagcagatgcactgtttgtgaaggtcctgcgatcgccatagccgttcacagccaaaccactgacattcctccatgtcctcacggctggatttctctctggaaaggattttcattcatcatgttcacaagtgcaggttctgagggcaccgggcaagcactggcctcccctggctcctgcctggaagaattccgagccagcccatttctagaatgtcatggaagaggaacgtgcaactactattcaaattcctacagtttctggctggcttcattaaacccagaaagaatgttcagaaagcctattccatcaactgtgaaagctggggaattagaaaaaataataagtcgctgtcaggtgtgcatgaagaaaagacactga

SEQ ID NO: 12 - protein, 1670 aa, Homo sapiens
MSARTAPRPQVLLLPLLLVLLAAAPAASKGCVCKDKGQCFCDGAKGEKGEKGFPGPPGSPGQKGFTGPEGLPGPQGPKGFPGLPGLTGSKGVRGISGLPGFSGSPGLPGTPGNTGPYGLVGVPGCSGSKGEQGFPGLPGTLGYPGIPGAAGLKGQKGAPAKEEDIELDAKGDPGLPGAPGPQGLPGPPGFPGPVGPPGPPGFFGFPGAMGPRGPKGHMGERVIGHKGERGVKGLTGPPGPPGTVIVTLTGPDNRTDLKGEKGDKGAMGEPGPPGPSGLPGESYGSEKGAPGDPGLQGKPGKDGVPGFPGSEGVKGNRGFPGLMGEDGIKGQKGDIGPPGFRGPTEYYDTYQEKGDEGTPGPPGPRGARGPQGPSGPPGVPGSPGSSRPGLRGAPGWPGLKGSKGERGRPGKDAMGTPGSPGCAGSPGLPGSPGPPGPPGDIVFRKGPPGDHGLPGYLGSPGIPGVDGPKGEPGLLCTQCPYIPGPPGLPGLPGLHGVKGIPGRQGAAGLKGSPGSPGNTGLPGFPGFPGAQGDPGLKGEKGETLQPEGQVGVPGDPGLRGQPGRKGLDGIPGTPGVKGLPGPKGELALSGEKGDQGPPGDPGSPGSPGPAGPAGPPGYGPQGEPGLQGTQGVPGAPGPPGEAGPRGELSVSTPVPGPPGPPGPPGHPGPQGPPGIPGSLGKCGDPGLPGPDGEPGIPGIGFPGPPGPKGDQGFPGTKGSLGCPGKMGEPGLPGKPGLPGAKGEPAVAMPGGPGTPGFPGERGNSGEHGEIGLPGLPGLPGTPGNEGLDGPRGDPGQPGPPGEQGPPGRCIEGPRGAQGLPGLNGLKGQQGRRGKTGPKGDPGIPGLDRSGFPGETGSPGIPGHQGEMGPLGQRGYPGNPGILGPPGEDGVIGMMGFPGAIGPPGPPGNPGTPGQRGSPGIPGVKGQRGTPGAKGEQGDKGNPGPSEISHVIGDKGEPGLKGFAGNPGEKGNRGVPGMPGLKGLKGLPGPAGPPGPRGDLGSTGNPGEPGLRGIPGSMGNMGMPGSKGKRGTLGFPGRAGRPGLPGIHGLQGDKGEPGYSEGTRPGPPGPTGDPGLPGDMGKKGEMGQPGPPGHLGPAGPEGAPGSPGSPGLPGKPGPHGDLGFKGIKGLLGPPGIRGPPGLPGFPGSPGPMGIRGDQGRDGIPGPAGEKGETGLLRAPPGPRGNPGAQGAKGDRGAPGFPGLPGRKGAMGDAGPRGPTGIEGFPGPPGLPGAIIPGQTGNRGPPGSRGSPGAPGPPGPPGSHVIGIKGDKGSMGHPGPKGPPGTAGDMGPPGRLGAPGTPGLPGPRGDPGFQGFPGVKGEKGNPGFLGSIGPPGPIGPKGPPGVRGDPGTLKIISLPGSPGPPGTPGEPGMQGEPGPPGPPGNLGPCGPRGKPGKDGKPGTPGPAGEKGNKGSKGEPGPAGSDGLPGLKGKRGDSGSPATWTTRGFVFTRHSQTTAIPSCPEGTVPLYSGFSFLFVQGNQRAHGQDLGTLGSCLQRFTTMPFLFCNVNDVCNFASRNDYSYWLSTPALMPMNMAPITGRALEPYISRCTVCEGPAIAIAVHSQTTDIPPCPHGWISLWKGFSFIMFTSAGSEGTGQALASPGSCLEEFRASPFLECHGRGTCNYYSNSYSFWLASLNPERMFRKPIPSTVKAGELEKIISRCQVCMKKRH

SEQ ID NO: 13 - DNA, 2781 bp, Homo sapiens
atggatatgtttcctctcacctgggttttcttagccctctacttttcaagacaccaagtgagaggccaaccagacccaccgtgcggaggtcgtttgaattccaaagatgctggctatatcacctctcccggttacccccaggactacccctcccaccagaactgcgagtggattgtttacgcccccgaacccaaccagaagattgtcctcaacttcaaccctcactttgaaatcgagaagcacgactgcaagtatgactttatcgagattcgggatggggacagtgaatccgcagacctcctgggcaaacactgtgggaacatcgccccgcccaccatcatctcctcgggctccatgctctacatcaagttcacctccgactacgcccggcagggggcaggcttctctctgcgctacgagatcttcaagacaggctctgaagattgctcaaaaaacttcacaagccccaacgggaccatcgaatctcctgggtttcctgagaagtatccacacaacttggactgcacctttaccatcctggccaaacccaagatggagatcatcctgcagttcctgatctttgacctggagcatgaccctttgcaggtgggagagggggactgcaagtacgattggctggacatctgggatggcattccacatgttggccccctgattggcaagtactgtgggaccaaaacaccctctgaacttcgttcatcgacggggatcctctccctgacctttcacacggacatggcggtggccaaggatggcttctctgcgcgttactacctggtccaccaagagccactagagaactttcagtgcaatgttcctctgggcatggagtctggccggattgctaatgaacagatcagtgcctcatctacctactctgatgggaggtggacccctcaacaaagccggctccatggtgatgacaatggctggacccccaacttggattccaacaaggagtatctccaggtggacctgcgctttttaaccatgctcacggccatcgcaacacagggagcgatttccagggaaacacagaatggctactatgtcaaatcctacaagctggaagtcagcactaatggagaggactggatggtgtaccggcatggcaaaaaccacaaggtatttcaagccaacaacgatgcaactgaggtggttctgaacaagctccacgctccactgctgacaaggtttgttagaatccgccctcagacctggcactcaggtatcgccctccggctggagctcttcggctgccgggtcacagatgctccctgctccaacatgctggggatgctctcaggcctcattgcagactcccagatctccgcctcttccacccaggaatacctctggagccccagtgcagcccgcctggtcagcagccgctcgggctggttccctcgaatccctcaggcccagcccggtgaggagtggcttcaggtagatctgggaacacccaagacagtgaaaggtgtcatcatccagggagcccgcggaggagacagtatcactgctgtggaagccagagcatttgtgcgcaagttcaaagtctcctacagcctaaacggcaaggactgggaatacattcaggaccccaggacccagcagccaaagctgttcgaagggaacatgcactatgacacccctgacatccgaaggtttgaccccattccggcacagtatgtgcgggtatacccggagaggtggtcgccggcggggattgggatgcggctggaggtgctgggctgtgactggacagactccaagcccacggtagagacgctgggacccactgtgaagagcgaagagacaaccaccccctaccccaccgaagaggaggccacagagtgtggggagaactgcagctttgaggatgacaaagatttgcagctcccttcgggattcaattgcaacttcgatttcctcgaggagccctgtggttggatgtatgaccatgccaagtggctccggaccacctgggccagcagctccagcccaaacgaccggacgtttccagatgacaggaatttcttgcggctgcagagtgacagccagagagagggccagtatgcccggctcatcagcccccctgtccacctgccccgaagcccggtgtgcatggagttccagtaccaggccacgggcggccgcggggtggcgctgcaggtggtgcgggaagccagccaggagagcaagttgctgtgggtcatccgtgaggaccagggcggcgagtggaagcacgggcggatcatcctgcccagctacgacatggagtaccagattgtgttcgagggagtgatagggaaaggacgttccggagagattgccattgatgacattcggataagcactgatgtcccactggagaactgcatggaacccatctcggcttttgcagtggacatcccagaaatacatgagagagaaggatatgaagatgaaattgatgatgaatacgaggtggactggagcaattcttcttctgcaacctcagggtctggcgccccctcgaccgacaaagaaaagagctggctgtacaccctggatcccatcctcatcaccatcatcgccatgagctcactgggcgtcctcctgggggccacctgtgcaggcctcctgctctactgcacctgttcctactcgggcctgagctcccgaagctgcaccacactggagaactacaacttcgagctctacgatggccttaagcacaaggtcaagatgaaccaccaaaagtgctgctccgaggcatga

SEQ ID NO: 14 - protein, 926 aa, Homo sapiens
MDMFPLTWVFLALYFSRHQVRGQPDPPCGGRLNSKDAGYITSPGYPQDYPSHQNCEWIVYAPEPNQKIVLNFNPHFEIEKHDCKYDFIEIRDGDSESADLLGKHCGNIAPPTIISSGSMLYIKFTSDYARQGAGFSLRYEIFKTGSEDCSKNFTSPNGTIESPGFPEKYPHNLDCTFTILAKPKMEIILQFLIFDLEHDPLQVGEGDCKYDWLDIWDGIPHVGPLIGKYCGTKTPSELRSSTGILSLTFHTDMAVAKDGFSARYYLVHQEPLENFQCNVPLGMESGRIANEQISASSTYSDGRWTPQQSRLHGDDNGWTPNLDSNKEYLQVDLRFLTMLTAIATQGAISRETQNGYYVKSYKLEVSTNGEDWMVYRHGKNHKVFQANNDATEVVLNKLHAPLLTRFVRIRPQTWHSGIALRLELFGCRVTDAPCSNMLGMLSGLIADSQISASSTQEYLWSPSAARLVSSRSGWFPRIPQAQPGEEWLQVDLGTPKTVKGVIIQGARGGDSITAVEARAFVRKFKVSYSLNGKDWEYIQDPRTQQPKLFEGNMHYDTPDIRRFDPIPAQYVRVYPERWSPAGIGMRLEVLGCDWTDSKPTVETLGPTVKSEETTTPYPTEEEATECGENCSFEDDKDLQLPSGFNCNFDFLEEPCGWMYDHAKWLRTTWASSSSPNDRTFPDDRNFLRLQSDSQREGQYARLISPPVHLPRSPVCMEFQYQATGGRGVALQVVREASQESKLLWVIREDQGGEWKHGRIILPSYDMEYQIVFEGVIGKGRSGEIAIDDIRISTDVPLENCMEPISAFAVDIPEIHEREGYEDEIDDEYEVDWSNSSSATSGSGAPSTDKEKSWLYTLDPILITIIAMSSLGVLLGATCAGLLLYCTCSYSGLSSRSCTTLENYNFELYDGLKHKVKMNHQKCCSEA

SEQ ID NO: 15 - DNA, 1173 bp, Homo sapiens
atggcgcaaaggtacgacgatctaccccattacgggggcatggatggagtaggcatcccctccacgatgtatggggacccgcatgcagccaggtccatgcagccggtccaccacctgaaccacgggcctcctctgcactcgcatcagtacccgcacacagctcataccaacgccatggcccccagcatgggctcctctgtcaatgacgctttaaagagagataaagatgccatttatggacaccccctcttccctctcttagcactgatttttgagaaatgtgaattagctacttgtaccccccgcgagccgggggtggcgggcggggacgtctgctcgtcagagtcattcaatgaagatatagccgtgttcgccaaacagattcgcgcagaaaaacctctattttcttctaatccagaactggataacttgatgattcaagccatacaagtattaaggtttcatctattggaattagagaaggtacacgaattatgtgacaatttctgccaccggtatattagctgtttgaaagggaaaatgcctatcgatttggtgatagacgatagagaaggaggatcaaaatcagacagtgaagatataacaagatcagcaaatctaactgaccagccctcttggaacagagatcatgatgacacggcatctactcgttcaggaggaaccccaggcccttccagcggtggccacacgtcacacagtggggacaacagcagtgagcaaggtgatggcttggacaacagtgtagcttcccccagcacaggtgacgatgatgaccctgataaggacaaaaagcgtcacaaaaagcgtggcatctttcccaaagtagccacaaatatcatgagggcgtggctgttccagcatctaacacacccttacccttctgaagaacagaaaaagcagttggcacaagacacgggactcaccatccttcaagtgaacaattggtttattaatgcccggagaagaatagtgcagcccatgatagaccagtccaaccgagcagtaagtcaaggaacaccttataatcctgatggacagcccatgggaggtttcgtaatggacggtcagcaacatatgggaattagagcaccaggacctatgagtggaatgggcatgaatatgggcatggaggggcagtggcactacatgtaa

SEQ ID NO: 16 - protein, 390 aa, Homo sapiens
MAQRYDDLPHYGGMDGVGIPSTMYGDPHAARSMQPVHHLNHGPPLHSHQYPHTAHTNAMAPSMGSSVNDALKRDKDAIYGHPLFPLLALIFEKCELATCTPREPGVAGGDVCSSESFNEDIAVFAKQIRAEKPLFSSNPELDNLMIQAIQVLRFHLLELEKVHELCDNFCHRYISCLKGKMPIDLVIDDREGGSKSDSEDITRSANLTDQPSWNRDHDDTASTRSGGTPGPSSGGHTSHSGDNSSEQGDGLDNSVASPSTGDDDDPDKDKKRHKKRGIFPKVATNIMRAWLFQHLTHPYPSEEQKKQLAQDTGLTILQVNNWFINARRRIVQPMIDQSNRAVSQGTPYNPDGQPMGGFVMDGQQHMGIRAPGPMSGMGMNMGMEGQWHYM

SEQ ID NO: 17 - DNA, 2097 bp, Homo sapiens
atgctgagcccaaagatcaggcaggccaggagggcccgctccaaaagcctagtgatgggggagcagagccggagccctgggcggatgccgtgccctcacaggctgggccccgtgctgaaggcgggctggctgaagaagcagaggagcatcatgaagaactggcagcagcgctggtttgtgctgcgtggggatcagcttttctactacaaggacaaagatgagatcaagccccagggatttatttctctacaagggacacaggtgactgaacttcctcctggccccgaggacccagggaagcacctctttgagatcagcccaggtggtgccggggagcgggagaaggtgccggccaaccccgaggcgctcctgctcatggccagctcccagcgtgacatggaggactgggtgcaggccatccgccgagtcatctgggccccgctgggcggagggatctttgggcagcgcctagaggaaacagtccaccacgagcggaagtatggcccccgcctggcgcccctgctggtggagcagtgtgtggacttcatccgggagcgcgggctcactgaggaggggctgttccgcatgccaggccaggccaacctggtgagggacctgcaggattccttcgactgtggggagaagccactgtttgacagcacaacagacgtgcacacggtggcctccctgctgaagctgtacctgcgggagctccccgagcccgtggtccccttcgccaggtacgaggacttcctcagctgcgcccagctgctcaccaaggacgagggggagggcactctggagttggctaaacaagtgagcaaccttcctcaggcaaattacaacctgctcagatacatctgcaagtttctggatgaagttcaggcatactcaaatgtcaacaagatgagtgtccagaatctggcaaccgtttttggacctaacattctgcggccacaggtagaggacccagtaaccatcatggaaggcacttccctcgtccagcacctgatgaccgtcctcatccgcaaacacagccagctcttcacggcaccggtcccggaagggcccacctccccgcgcgggggcctgcaatgcgcagtggggtggggctccgaggaggtcaccagggacagccaaggagagcccggcggccccggcctgcccgcgcacaggacctcttccctggacggggcggccgtggcggtgctctccagaacagcccccacggggccggggagccggtgcagccctgggaagaaggtgcagaccctgcccagttggaagtcctccttccggcagccgaggtccctatcgggaagcccgaaggggggcggctcatccctggaggtgcccatcatctcctccggcgggaactggcttatgaacgggctgtcctccctgcgcggacaccgccgggcctcgtcgggagaccggctcaaggactcgggctccgtgcagagactctccacctacgacaatgtgcccgcgccgggcctggtccccggcatacccagcgtggccagtatggcgtggtccggggcctcgtccagcgagtcgtcggtggggggctcactcagcagctgcacggcctgccgcgccagcgactcgtctgcccgcagttccctgcacaccgactgggccctggagccctccccgctccccagcagcagcgaggaccccaagtccctggacctggaccacagcatggacgaggcgggcgcgggtgccagcaacagcgagcccagcgagccggacagccccacccgggaacacgcgcgccgctccgaggccttacaggggctggtcactgagctcagggccgagctgtgccgccagcggactgagtacgagaggagtgtgaaaagaatcgaagaagggagtgctgacctgagaaaacgaatgtcccggttagaagaagaactggaccaggaaaagaaaaaatacatcatgctggaaataaagctgcggaactctgaacgggcgcgggaggatgcggagaggaggaaccagctgttgcagagggaaatggaggagtttttttcgaccctaggaagcttgactgttggggcaaaaggtgccagggccccaaagtaa

SEQ ID NO: 18 - protein, 698 aa, Homo sapiens
MLSPKIRQARRARSKSLVMGEQSRSPGRMPCPHRLGPVLKAGWLKKQRSIMKNWQQRWFVLRGDQLFYYKDKDEIKPQGFISLQGTQVTELPPGPEDPGKHLFEISPGGAGEREKVPANPEALLLMASSQRDMEDWVQAIRRVIWAPLGGGIFGQRLEETVHHERKYGPRLAPLLVEQCVDFIRERGLTEEGLFRMPGQANLVRDLQDSFDCGEKPLFDSTTDVHTVASLLKLYLRELPEPVVPFARYEDFLSCAQLLTKDEGEGTLELAKQVSNLPQANYNLLRYICKFLDEVQAYSNVNKMSVQNLATVFGPNILRPQVEDPVTIMEGTSLVQHLMTVLIRKHSQLFTAPVPEGPTSPRGGLQCAVGWGSEEVTRDSQGEPGGPGLPAHRTSSLDGAAVAVLSRTAPTGPGSRCSPGKKVQTLPSWKSSFRQPRSLSGSPKGGGSSLEVPIISSGGNWLMNGLSSLRGHRRASSGDRLKDSGSVQRLSTYDNVPAPGLVPGIPSVASMAWSGASSSESSVGGSLSSCTACRASDSSARSSLHTDWALEPSPLPSSSEDPKSLDLDHSMDEAGAGASNSEPSEPDSPTREHARRSEALQGLVTELRAELCRQRTEYERSVKRIEEGSADLRKRMSRLEEELDQEKKKYIMLEIKLRNSERAREDAERRNQLLQREMEEFFSTLGSLTVGAKGARAPK

SEQ ID NO: 19 - DNA, 19 bp, Artificial
  Synthetic DNA
agtcgcctgtcaccatttc

SEQ ID NO: 20 - DNA, 20 bp, Artificial
  Synthetic DNA
actacggacattttcgcatc

SEQ ID NO: 21 - DNA, 22 bp, Artificial
  Synthetic DNA
gtttgattgctgcatatttcag

SEQ ID NO: 22 - DNA, 23 bp, Artificial
  Synthetic DNA
ggcttagaaatcttttctaaatg

SEQ ID NO: 23 - DNA, 21 bp, Artificial
  Synthetic DNA
aatgacatgattactactcta

SEQ ID NO: 24 - DNA, 21 bp, Artificial
  Synthetic DNA
ttaatcggtttaggaatacaa

SEQ ID NO: 25 - DNA, 22 bp, Artificial
  Synthetic DNA
cattataaagattcaggcaatg

SEQ ID NO: 26 - DNA, 21 bp, Artificial
  Synthetic DNA
gacagtaagatacagtctatc

SEQ ID NO: 27 - DNA, 22 bp, Artificial
  Synthetic DNA
acctgttaagtttgtatgcaac

SEQ ID NO: 28 - DNA, 20 bp, Artificial
  Synthetic DNA
tccaggaagaggaaaggaaa

SEQ ID NO: 29 - DNA, 23 bp, Artificial
  Synthetic DNA
catagcaatttagtgaaataact

SEQ ID NO: 30 - DNA, 22 bp, Artificial
  Synthetic DNA
gatatggttaagaaaactgttc

SEQ ID NO: 31 - DNA, 21 bp, Artificial
  Synthetic DNA
tgacagtttgacagttaaagg

SEQ ID NO: 32 - DNA, 21 bp, Artificial
  Synthetic DNA
ggatatttctcccaatgaaag

SEQ ID NO: 33 - DNA, 21 bp, Artificial
  Synthetic DNA
ctcagattgccttataatagt

SEQ ID NO: 34 - DNA, 21 bp, Artificial
  Synthetic DNA
tcatgttactgctacgtaaac

SEQ ID NO: 35 - DNA, 20 bp, Artificial
  Synthetic DNA
aaggcctcttaaagatcatg

SEQ ID NO: 36 - DNA, 21 bp, Artificial
  Synthetic DNA
ttttcatggtgttttatccct

SEQ ID NO: 37 - DNA, 23 bp, Artificial
  Synthetic DNA
tctgccatctctctcctcctttt

SEQ ID NO: 38 - DNA, 24 bp, Artificial
  Synthetic DNA
tctgacacaatgtcctattgccat

SEQ ID NO: 39 - DNA, 24 bp, Artificial
  Synthetic DNA
gccccggctctcggttataagatg

SEQ ID NO: 40 - DNA, 20 bp, Artificial
  Synthetic DNA
ccgttccccagagattccaa

SEQ ID NO: 41 - DNA, 20 bp, Artificial
  Synthetic DNA
tgccattccggaggaggtgt

SEQ ID NO: 42 - DNA, 20 bp, Artificial
  Synthetic DNA
gcccatcaggaatctcccaa

SEQ ID NO: 43 - DNA, 21 bp, Artificial
  Synthetic DNA
atctggatcatccccttccgc

SEQ ID NO: 44 - DNA, 26 bp, Artificial
  Synthetic DNA
cccggaacagaaagtaaagcctctag

SEQ ID NO: 45 - DNA, 1497 bp, Homo sapiens
atgacagttttcctttcctttgctttcctcgctgccattctgactcacatagggtgcagcaatcagcgccgaagtccagaaaacagtgggagaagatataaccggattcaacatgggcaatgtgcctacactttcattcttccagaacacgatggcaactgtcgtgagagtacgacagaccagtacaacacaaacgctctgcagagagatgctccacacgtggaaccggatttctcttcccagaaacttcaacatctggaacatgtgatggaaaattatactcagtggctgcaaaaacttgagaattacattgtggaaaacatgaagtcggagatggcccagatacagcagaatgcagttcagaaccacacggctaccatgctggagataggaaccagcctcctctctcagactgcagagcagaccagaaagctgacagatgttgagacccaggtactaaatcaaacttctcgacttgagatacagctgctggagaattcattatccacctacaagctagagaagcaacttcttcaacagacaaatgaaatcttgaagatccatgaaaaaaacagtttattagaacataaaatcttagaaatggaaggaaaacacaaggaagagttggacaccttaaaggaagagaaagagaaccttcaaggcttggttactcgtcaaacatatataatccaggagctggaaaagcaattaaacagagctaccaccaacaacagtgtccttcagaagcagcaactggagctgatggacacagtccacaaccttgtcaatctttgcactaaagaaggtgttttactaaagggaggaaaaagagaggaagagaaaccatttagagactgtgcagatgtatatcaagctggttttaataaaagtggaatctacactatttatattaataatatgccagaacccaaaaaggtgttttgcaatatggatgtcaatgggggaggttggactgtaatacaacatcgtgaagatggaagtctagatttccaaagaggctggaaggaatataaaatgggttttggaaatccctccggtgaatattggctggggaatgagtttatttttgccattaccagtcagaggcagtacatgctaagaattgagttaatggactgggaagggaaccgagcctattcacagtatgacagattccacataggaaatgaaaagcaaaactataggttgtatttaaaaggtcacactgggacagcaggaaaacagagcagcctgatcttacacggtgctgatttcagcactaaagatgctgataatgacaactgtatgtgcaaatgtgccctcatgttaacaggaggatggtggtttgatgcttgtggcccctccaatctaaatggaatgttctatactgcgggacaaaaccatggaaaactgaatgggataaagtggcactacttcaaagggcccagttactccttacgttccacaactatgatgattcgacctttagatttttga

SEQ ID NO: 46 - protein, 498 aa, Homo sapiens
MTVFLSFAFLAAILTHIGCSNQRRSPENSGRRYNRIQHGQCAYTFILPEHDGNCRESTTDQYNTNALQRDAPHVEPDFSSQKLQHLEHVMENYTQWLQKLENYIVENMKSEMAQIQQNAVQNHTATMLEIGTSLLSQTAEQTRKLTDVETQVLNQTSRLEIQLLENSLSTYKLEKQLLQQTNEILKIHEKNSLLEHKILEMEGKHKEELDTLKEEKENLQGLVTRQTYIIQELEKQLNRATTNNSVLQKQQLELMDTVHNLVNLCTKEGVLLKGGKREEEKPFRDCADVYQAGFNKSGIYTIYINNMPEPKKVFCNMDVNGGGWTVIQHREDGSLDFQRGWKEYKMGFGNPSGEYWLGNEFIFAITSQRQYMLRIELMDWEGNRAYSQYDRFHIGNEKQNYRLYLKGHTGTAGKQSSLILHGADFSTKDADNDNCMCKCALMLTGGWWFDACGPSNLNGMFYTAGQNHGKLNGIKWHYFKGPSYSLRSTTMMIRPLDF

SEQ ID NO: 47 - DNA, 1335 bp, Homo sapiens
atgtggcagattgttttctttactctgagctgtgatcttgtcttggccgcagcctataacaactttcggaagagcatggacagcataggaaagaagcaatatcaggtccagcatgggtcctgcagctacactttcctcctgccagagatggacaactgccgctcttcctccagcccctacgtgtccaatgctgtgcagagggacgcgccgctcgaatacgatgactcggtgcagaggctgcaagtgctggagaacatcatggaaaacaacactcagtggctaatgaaggtattaaatcagaccacgagacttgaacttcagctcttggaacactccctctcgacaaacaaattggaaaaacagattttggaccagaccagtgaaataaacaaattgcaagataagaacagtttcctagaaaagaaggtgctagctatggaagacaagcacatcatccaactacagtcaataaaagaagagaaagatcagctacaggtgttagtatccaagcaaaattccatcattgaagaactagaaaaaaaaatagtgactgccacggtgaataattcagttcttcagaagcagcaacatgatctcatggagacagttaataacttactgactatgatgtccacatcaaactcagctaaggaccccactgttgctaaagaagaacaaatcagcttcagagactgtgctgaagtattcaaatcaggacacaccacgaatggcatctacacgttaacattccctaattctacagaagagatcaaggcctactgtgacatggaagctggaggaggcgggtggacaattattcagcgacgtgaggatggcagcgttgattttcagaggacttggaaagaatataaagtgggatttggtaacccttcaggagaatattggctgggaaatgagtttgtttcgcaactgactaatcagcaacgctatgtgcttaaaatacaccttaaagactgggaagggaatgaggcttactcattgtatgaacatttctatctctcaagtgaagaactcaattataggattcaccttaaaggacttacagggacagccggcaaaataagcagcatcagccaaccaggaaatgattttagcacaaaggatggagacaacgacaaatgtatttgcaaatgttcacaaatgctaacaggaggctggtggtttgatgcatgtggtccttccaacttgaacggaatgtactatccacagaggcagaacacaaataagttcaacggcattaaatggtactactggaaaggctcaggctattcgctcaaggccacaaccatgatgatccgaccagcagatttctaa

SEQ ID NO: 48 - protein, 444 aa, Homo sapiens
MWQIVFFTLSCDLVLAAAYNNFRKSMDSIGKKQYQVQHGSCSYTFLLPEMDNCRSSSSPYVSNAVQRDAPLEYDDSVQRLQVLENIMENNTQWLMKVLNQTTRLELQLLEHSLSTNKLEKQILDQTSEINKLQDKNSFLEKKVLAMEDKHIIQLQSIKEEKDQLQVLVSKQNSIIEELEKKIVTATVNNSVLQKQQHDLMETVNNLLTMMSTSNSAKDPTVAKEEQISFRDCAEVFKSGHTTNGIYTLTFPNSTEEIKAYCDMEAGGGGWTIIQRREDGSVDFQRTWKEYKVGFGNPSGEYWLGNEFVSQLTNQQRYVLKIHLKDWEGNEAYSLYEHFYLSSEELNYRIHLKGLTGTAGKISSISQPGNDFSTKDGDNDKCICKCSQMLTGGWWFDACGPSNLNGMYYPQRQNTNKFNGIKWYYWKGSGYSLKATTMMIRPADF

SEQ ID NO: 49 - DNA, 1854 bp, Homo sapiens
atggctgaagcaaagacccactggcttggagcagccctgtctcttatccctttaattttcctcatctctggggctgaagcagcttcatttcagagaaaccagctgcttcagaaagaaccagacctcaggttggaaaatgtccaaaagtttcccagtcctgaaatgatcagggctttggagtacatagaaaacctccgacaacaagctcataaggaagaaagcagcccagattataatccctaccaaggtgtctctgtcccccttcagcaaaaagaaaatggcgatgaaagccacttgcccgagagggattcactgagtgaagaagactggatgagaataatactcgaagctttgagacaggctgaaaatgagcctcagtctgcaccaaaagaaaataagccctatgccttgaattcagaaaagaactttccaatggacatgagtgatgattatgagacacagcagtggccagaaagaaagcttaagcacatgcaattccctcctatgtatgaagagaattccagggataacccctttaaacgcacaaatgaaatagtggaggaacaatatactcctcaaagccttgctacattggaatctgtcttccaagagctggggaaactgacaggaccaaacaaccagaaacgtgagaggatggatgaggagcaaaaactttatacggatgatgaagatgatatctacaaggctaataacattgcctatgaagatgtggtcgggggagaagactggaacccagtagaggagaaaatagagagtcaaacccaggaagaggtgagagacagcaaagagaatatagaaaaaaatgaacaaatcaacgatgagatgaaacgctcagggcagcttggcatccaggaagaagatcttcggaaagagagtaaagaccaactctcagatgatgtctccaaagtaattgcctatttgaaaaggttagtaaatgctgcaggaagtgggaggttacagaatgggcaaaatggggaaagggccaccaggctttttgagaaacctcttgattctcagtctatttatcagctgattgaaatctcaaggaatttacagatacccccagaagacttaattgagatgctcaaaactggggagaagccgaatggatcagtggaaccggagcgggagcttgaccttcctgttgacctagatgacatctcagaggctgacttagaccatccagacctgttccaaaataggatgctctccaagagtggctaccctaaaacacctggtcgtgctgggactgaggccctaccagacgggctcagtgttgaggatattttaaatcttttagggatggagagtgcagcaaatcagaaaacgtcgtattttcccaatccatataaccaggagaaagttctgccaaggctcccttatggtgctggaagatctagatcgaaccagcttcccaaagctgcctggattccacatgttgaaaacagacagatggcatatgaaaacctgaacgacaaggatcaagaattaggtgagtacttggccaggatgctagttaaataccctgagatcattaattcaaaccaagtgaagcgagttcctggtcaaggctcatctgaagatgacctgcaggaagaggaacaaattgagcaggccatcaaagagcatttgaatcaaggcagctctcaggagactgacaagctggccccggtgagcaaaaggttccctgtggggcccccgaagaatgatgataccccaaataggcagtactgggatgaagatctgttaatgaaagtgctggaatacctcaaccaagaaaaggcagaaaagggaagggagcatattgctaagagagcaatggaaaatatgtaa

SEQ ID NO: 50 - protein, 617 aa, Homo sapiens
MAEAKTHWLGAALSLIPLIFLISGAEAASFQRNQLLQKEPDLRLENVQKFPSPEMIRALEYIENLRQQAHKEESSPDYNPYQGVSVPLQQKENGDESHLPERDSLSEEDWMRIILEALRQAENEPQSAPKENKPYALNSEKNFPMDMSDDYETQQWPERKLKHMQFPPMYEENSRDNPFKRTNEIVEEQYTPQSLATLESVFQELGKLTGPNNQKRERMDEEQKLYTDDEDDIYKANNIAYEDVVGGEDWNPVEEKIESQTQEEVRDSKENIEKNEQINDEMKRSGQLGIQEEDLRKESKDQLSDDVSKVIAYLKRLVNAAGSGRLQNGQNGERATRLFEKPLDSQSIYQLIEISRNLQIPPEDLIEMLKTGEKPNGSVEPERELDLPVDLDDISEADLDHPDLFQNRMLSKSGYPKTPGRAGTEALPDGLSVEDILNLLGMESAANQKTSYFPNPYNQEKVLPRLPYGAGRSRSNQLPKAAWIPHVENRQMAYENLNDKDQELGEYLARMLVKYPEIINSNQVKRVPGQGSSEDDLQEEEQIEQAIKEHLNQGSSQETDKLAPVSKRFPVGPPKNDDTPNRQYWDEDLLMKVLEYLNQEKAEKGREHIAKRAMENM

SEQ ID NO: 51 - DNA, 627 bp, Homo sapiens
atggctcccttaggtgaagttgggaactatttcggtgtgcaggatgcggtaccgtttgggaatgtgcccgtgttgccggtggacagcccggttttgttaagtgaccacctgggtcagtccgaagcaggggggctccccaggggacccgcagtcacggacttggatcatttaaaggggattctcaggcggaggcagctatactgcaggactggatttcacttagaaatcttccccaatggtactatccagggaaccaggaaagaccacagccgatttggcattctggaatttatcagtatagcagtgggcctggtcagcattcgaggcgtggacagtggactctacctcgggatgaatgagaagggggagctgtatggatcagaaaaactaacccaagagtgtgtattcagagaacagttcgaagaaaactggtataatacgtactcatcaaacctatataagcacgtggacactggaaggcgatactatgttgcattaaataaagatgggaccccgagagaagggactaggactaaacggcaccagaaattcacacattttttacctagaccagtggaccccgacaaagtacctgaactgtataaggatattctaagccaaagttga

SEQ ID NO: 52 - protein, 208 aa, Homo sapiens
MAPLGEVGNYFGVQDAVPFGNVPVLPVDSPVLLSDHLGQSEAGGLPRGPAVTDLDHLKGILRRRQLYCRTGFHLEIFPNGTIQGTRKDHSRFGILEFISIAVGLVSIRGVDSGLYLGMNEKGELYGSEKLTQECVFREQFEENWYNTYSSNLYKHVDTGRRYYVALNKDGTPREGTRTKRHQKFTHFLPRPVDPDKVPELYKDILSQS

SEQ ID NO: 53 - DNA, 1902 bp, Homo sapiens
atggagcctgcacccgcccgatctccgaggccccagcaggaccccgcccggccccaggagcccaccatgcctccccccgagaccccctctgaaggccgccagcccagccccagccccagccctacagagcgagcccccgcttcggaggaggagttccagtttctgcgctgccagcaatgccaggcggaagccaagtgcccgaagctgctgccttgtctgcacacgctgtgctcaggatgcctggaggcgtcgggcatgcagtgccccatctgccaggcgccctggcccctaggtgcagacacacccgccctggataacgtctttttcgagagtctgcagcggcgcctgtcggtgtaccggcagattgtggatgcgcaggctgtgtgcacccgctgcaaagagtcggccgacttctggtgctttgagtgcgagcagctcctctgcgccaagtgcttcgaggcacaccagtggttcctcaagcacgaggcccggcccctagcagagctgcgcaaccagtcggtgcgtgagttcctggacggcacccgcaagaccaacaacatcttctgctccaaccccaaccaccgcacccctacgctgaccagcatctactgccgaggatgttccaagccgctgtgctgctcgtgcgcgctccttgacagcagccacagtgagctcaagtgcgacatcagcgcagagatccagcagcgacaggaggagctggacgccatgacgcaggcgctgcaggagcaggatagtgcctttggcgcggttcacgcgcagatgcacgcggccgtcggccagctgggccgcgcgcgtgccgagaccgaggagctgatccgcgagcgcgtgcgccaggtggtagctcacgtgcgggctcaggagcgcgagctgctggaggctgtggacgcgcggtaccagcgcgactacgaggagatggccagtcggctgggccgcctggatgctgtgctgcagcgcatccgcacgggcagcgcgctggtgcagaggatgaagtgctacgcctcggaccaggaggtgctggacatgcacggtttcctgcgccaggcgctctgccgcctgcgccaggaggagccccagagcctgcaagctgccgtgcgcaccgatggcttcgacgagttcaaggtgcgcctgcaggacctcagctcttgcatcacccaggggaaagatgcagctgtatccaagaaagccagcccagaggctgccagcactcccagggaccctattgacgttgacctgcccgaggaggcagagagagtgaaggcccaggttcaggccctggggctggctgaagcccagcctatggctgtggtacagtcagtgcccggggcacaccccgtgccagtgtacgccttctccatcaaaggcccttcctatggagaggatgtctccaatacaacgacagcccagaagaggaagtgcagccagacccagtgccccaggaaggtcatcaagatggagtctgaggaggggaaggaggcaaggttggctcggagctccccggagcagcccaggcccagcacctccaaggcagtctcaccaccccacctggatggaccgcctagccccaggagccccgtcataggaagtgaggtcttcctgcccaacagcaaccacgtggccagtggcgccggggaggcagaggaacgcgttgtggtgatcagcagctcggaagactcagatgccgaaaactcgtcctcccgagagctggatgacagcagcagtgagtccagtgacctccagctggaaggccccagcaccctcagggtcctggacgagaaccttgctgacccccaagcagaagacagacctctggttttctttgacctcaagattgacaatgaaagtgggttctcctggggctacccccacccctttctaatttag

SEQ ID NO: 54 - protein, 633 aa, Homo sapiens
MEPAPARSPRPQQDPARPQEPTMPPPETPSEGRQPSPSPSPTERAPASEEEFQFLRCQQCQAEAKCPKLLPCLHTLCSGCLEASGMQCPICQAPWPLGADTPALDNVFFESLQRRLSVYRQIVDAQAVCTRCKESADFWCFECEQLLCAKCFEAHQWFLKHEARPLAELRNQSVREFLDGTRKTNNIFCSNPNHRTPTLTSIYCRGCSKPLCCSCALLDSSHSELKCDISAEIQQRQEELDAMTQALQEQDSAFGAVHAQMHAAVGQLGRARAETEELIRERVRQVVAHVRAQERELLEAVDARYQRDYEEMASRLGRLDAVLQRIRTGSALVQRMKCYASDQEVLDMHGFLRQALCRLRQEEPQSLQAAVRTDGFDEFKVRLQDLSSCITQGKDAAVSKKASPEAASTPRDPIDVDLPEEAERVKAQVQALGLAEAQPMAVVQSVPGAHPVPVYAFSIKGPSYGEDVSNTTTAQKRKCSQTQCPRKVIKMESEEGKEARLARSSPEQPRPSTSKAVSPPHLDGPPSPRSPVIGSEVFLPNSNHVASGAGEAEERVVVISSSEDSDAENSSSRELDDSSSESSDLQLEGPSTLRVLDENLADPQAEDRPLVFFDLKIDNESGFSWGYPHPFLI

SEQ ID NO: 55 - DNA, 2421 bp, Homo sapiens
atgggcgcccctgcctgcgccctcgcgctctgcgtggccgtggccatcgtggccggcgcctcctcggagtccttggggacggagcagcgcgtcgtggggcgagcggcagaagtcccgggcccagagcccggccagcaggagcagttggtcttcggcagcggggatgctgtggagctgagctgtcccccgcccgggggtggtcccatggggcccactgtctgggtcaaggatggcacagggctggtgccctcggagcgtgtcctggtggggccccagcggctgcaggtgctgaatgcctcccacgaggactccggggcctacagctgccggcagcggctcacgcagcgcgtactgtgccacttcagtgtgcgggtgacagacgctccatcctcgggagatgacgaagacggggaggacgaggctgaggacacaggtgtggacacaggggccccttactggacacggcccgagcggatggacaagaagctgctggccgtgccggccgccaacaccgtccgcttccgctgcccagccgctggcaaccccactccctccatctcctggctgaagaacggcagggagttccgcggcgagcaccgcattggaggcatcaagctgcggcatcagcagtggagcctggtcatggaaagcgtggtgccctcggaccgcggcaactacacctgcgtcgtggagaacaagtttggcagcatccggcagacgtacacgctggacgtgctggagcgctccccgcaccggcccatcctgcaggcggggctgccggccaaccagacggcggtgctgggcagcgacgtggagttccactgcaaggtgtacagtgacgcacagccccacatccagtggctcaagcacgtggaggtgaatggcagcaaggtgggcccggacggcacaccctacgttaccgtgctcaagacggcgggcgctaacaccaccgacaaggagctagaggttctctccttgcacaacgtcacctttgaggacgccggggagtacacctgcctggcgggcaattctattgggttttctcatcactctgcgtggctggtggtgctgccagccgaggaggagctggtggaggctgacgaggcgggcagtgtgtatgcaggcatcctcagctacggggtgggcttcttcctgttcatcctggtggtggcggctgtgacgctctgccgcctgcgcagcccccccaagaaaggcctgggctcccccaccgtgcacaagatctcccgcttcccgctcaagcgacaggtgtccctggagtccaacgcgtccatgagctccaacacaccactggtgcgcatcgcaaggctgtcctcaggggagggccccacgctggccaatgtctccgagctcgagctgcctgccgaccccaaatgggagctgtctcgggcccggctgaccctgggcaagccccttggggagggctgcttcggccaggtggtcatggcggaggccatcggcattgacaaggaccgggccgccaagcctgtcaccgtagccgtgaagatgctgaaagacgatgccactgacaaggacctgtcggacctggtgtctgagatggagatgatgaagatgatcgggaaacacaaaaacatcatcaacctgctgggcgcctgcacgcagggcgggcccctgtacgtgctggtggagtacgcggccaagggtaacctgcgggagtttctgcgggcgcggcggcccccgggcctggactactccttcgacacctgcaagccgcccgaggagcagctcaccttcaaggacctggtgtcctgtgcctaccaggtggcccggggcatggagtacttggcctcccagaagtgcatccacagggacctggctgcccgcaatgtgctggtgaccgaggacaacgtgatgaagatcgcagacttcgggctggcccgggacgtgcacaacctcgactactacaagaagacgaccaacggccggctgcccgtgaagtggatggcgcctgaggccttgtttgaccgagtctacactcaccagagtgacgtctggtcctttggggtcctgctctgggagatcttcacgctggggggctccccgtaccccggcatccctgtggaggagctcttcaagctgctgaaggagggccaccgcatggacaagcccgccaactgcacacacgacctgtacatgatcatgcgggagtgctggcatgccgcgccctcccagaggcccaccttcaagcagctggtggaggacctggaccgtgtccttaccgtgacgtccaccgacgagtacctggacctgtcggcgcctttcgagcagtactccccgggtggccaggacacccccagctccagctcctcaggggacgactccgtgtttgcccacgacctgctgcccccggccccacccagcagtgggggctcgcggacgtga

SEQ ID NO: 56 - protein, 806 aa, Homo sapiens
MGAPACALALCVAVAIVAGASSESLGTEQRVVGRAAEVPGPEPGQQEQLVFGSGDAVELSCPPPGGGPMGPTVWVKDGTGLVPSERVLVGPQRLQVLNASHEDSGAYSCRQRLTQRVLCHFSVRVTDAPSSGDDEDGEDEAEDTGVDTGAPYWTRPERMDKKLLAVPAANTVRFRCPAAGNPTPSISWLKNGREFRGEHRIGGIKLRHQQWSLVMESVVPSDRGNYTCVVENKFGSIRQTYTLDVLERSPHRPILQAGLPANQTAVLGSDVEFHCKVYSDAQPHIQWLKHVEVNGSKVGPDGTPYVTVLKTAGANTTDKELEVLSLHNVTFEDAGEYTCLAGNSIGFSHHSAWLVVLPAEEELVEADEAGSVYAGILSYGVGFFLFILVVAAVTLCRLRSPPKKGLGSPTVHKISRFPLKRQVSLESNASMSSNTPLVRIARLSSGEGPTLANVSELELPADPKWELSRARLTLGKPLGEGCFGQVVMAEAIGIDKDRAAKPVTVAVKMLKDDATDKDLSDLVSEMEMMKMIGKHKNIINLLGACTQGGPLYVLVEYAAKGNLREFLRARRPPGLDYSFDTCKPPEEQLTFKDLVSCAYQVARGMEYLASQKCIHRDLAARNVLVTEDNVMKIADFGLARDVHNLDYYKKTTNGRLPVKWMAPEALFDRVYTHQSDVWSFGVLLWEIFTLGGSPYPGIPVEELFKLLKEGHRMDKPANCTHDLYMIMRECWHAAPSQRPTFKQLVEDLDRVLTVTSTDEYLDLSAPFEQYSPGGQDTPSSSSSGDDSVFAHDLLPPAPPSSGGSRT

SEQ ID NO: 57 - DNA, 2115 bp, Homo sapiens
atggtcagctggggtcgtttcatctgcctggtcgtggtcaccatggcaaccttgtccctggcccggccctccttcagtttagttgaggataccacattagagccagaaggagcaccatactggaccaacacagaaaagatggaaaagcggctccatgctgtgcctgcggccaacactgtcaagtttcgctgcccagccggggggaacccaatgccaaccatgcggtggctgaaaaacgggaaggagtttaagcaggagcatcgcattggaggctacaaggtacgaaaccagcactggagcctcattatggaaagtgtggtcccatctgacaagggaaattatacctgtgtagtggagaatgaatacgggtccatcaatcacacgtaccacctggatgttgtggagcgatcgcctcaccggcccatcctccaagccggactgccggcaaatgcctccacagtggtcggaggagacgtagagtttgtctgcaaggtttacagtgatgcccagccccacatccagtggatcaagcacgtggaaaagaacggcagtaaatacgggcccgacgggctgccctacctcaaggttctcaaggccgccggtgttaacaccacggacaaagagattgaggttctctatattcggaatgtaacttttgaggacgctggggaatatacgtgcttggcgggtaattctattgggatatcctttcactctgcatggttgacagttctgccagcgcctggaagagaaaaggagattacagcttccccagactacctggagatagccatttactgcataggggtcttcttaatcgcctgtatggtggtaacagtcatcctgtgccgaatgaagaacacgaccaagaagccagacttcagcagccagccggctgtgcacaagctgaccaaacgtatccccctgcggagacaggtttcggctgagtccagctcctccatgaactccaacaccccgctggtgaggataacaacacgcctctcttcaacggcagacacccccatgctggcaggggtctccgagtatgaacttccagaggacccaaaatgggagtttccaagagataagctgacactgggcaagcccctgggagaaggttgctttgggcaagtggtcatggcggaagcagtgggaattgacaaagacaagcccaaggaggcggtcaccgtggccgtgaagatgttgaaagatgatgccacagagaaagacctttctgatctggtgtcagagatggagatgatgaagatgattgggaaacacaagaatatcataaatcttcttggagcctgcacacaggatgggcctctctatgtcatagttgagtatgcctctaaaggcaacctccgagaatacctccgagcccggaggccacccgggatggagtactcctatgacattaaccgtgttcctgaggagcagatgaccttcaaggacttggtgtcatgcacctaccagctggccagaggcatggagtacttggcttcccaaaaatgtattcatcgagatttagcagccagaaatgttttggtaacagaaaacaatgtgatgaaaatagcagactttggactcgccagagatatcaacaatatagactattacaaaaagaccaccaatgggcggcttccagtcaagtggatggctccagaagccctgtttgatagagtatacactcatcagagtgatgtctggtccttcggggtgttaatgtgggagatcttcactttagggggctcgccctacccagggattcccgtggaggaactttttaagctgctgaaggaaggacacagaatggataagccagccaactgcaccaacgaactgtacatgatgatgagggactgttggcatgcagtgccctcccagagaccaacgttcaagcagttggtagaagacttggatcgaattctcactctcacaaccaatgaggaatacttggacctcagccaacctctcgaacagtattcacctagttaccctgacacaagaagttcttgttcttcaggagatgattctgttttttctccagaccccatgccttacgaaccatgccttcctcagtatccacacataaacggcagtgttaaaacatga

SEQ ID NO: 58 - protein, 704 aa, Homo sapiens
MVSWGRFICLVVVTMATLSLARPSFSLVEDTTLEPEGAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVERSPHRPILQAGLPANASTVVGGDVEFVCKVYSDAQPHIQWIKHVEKNGSKYGPDGLPYLKVLKAAGVNTTDKEIEVLYIRNVTFEDAGEYTCLAGNSIGISFHSAWLTVLPAPGREKEITASPDYLEIAIYCIGVFLIACMVVTVILCRMKNTTKKPDFSSQPAVHKLTKRIPLRRQVSAESSSSMNSNTPLVRITTRLSSTADTPMLAGVSEYELPEDPKWEFPRDKLTLGKPLGEGCFGQVVMAEAVGIDKDKPKEAVTVAVKMLKDDATEKDLSDLVSEMEMMKMIGKHKNIINLLGACTQDGPLYVIVEYASKGNLREYLRARRPPGMEYSYDINRVPEEQMTFKDLVSCTYQLARGMEYLASQKCIHRDLAARNVLVTENNVMKIADFGLARDINNIDYYKKTTNGRLPVKWMAPEALFDRVYTHQSDVWSFGVLMWEIFTLGGSPYPGIPVEELFKLLKEGHRMDKPANCTNELYMMMRDCWHAVPSQRPTFKQLVEDLDRILTLTTNEEYLDLSQPLEQYSPSYPDTRSSCSSGDDSVFSPDPMPYEPCLPQYPHINGSVKT

SEQ ID NO: 59 - DNA, 2463 bp, Homo sapiens
atgtggagctggaagtgcctcctcttctgggctgtgctggtcacagccacactctgcaccgctaggccgtccccgaccttgcctgaacaagcccagccctggggagcccctgtggaagtggagtccttcctggtccaccccggtgacctgctgcagcttcgctgtcggctgcgggacgatgtgcagagcatcaactggctgcgggacggggtgcagctggcggaaagcaaccgcacccgcatcacaggggaggaggtggaggtgcaggactccgtgcccgcagactccggcctctatgcttgcgtaaccagcagcccctcgggcagtgacaccacctacttctccgtcaatgtttcagatgctctcccctcctcggaggatgatgatgatgatgatgactcctcttcagaggagaaagaaacagataacaccaaaccaaaccgtatgcccgtagctccatattggacatccccagaaaagatggaaaagaaattgcatgcagtgccggctgccaagacagtgaagttcaaatgcccttccagtgggaccccaaaccccacactgcgctggttgaaaaatggcaaagaattcaaacctgaccacagaattggaggctacaaggtccgttatgccacctggagcatcataatggactctgtggtgccctctgacaagggcaactacacctgcattgtggagaatgagtacggcagcatcaaccacacataccagctggatgtcgtggagcggtcccctcaccggcccatcctgcaagcagggttgcccgccaacaaaacagtggccctgggtagcaacgtggagttcatgtgtaaggtgtacagtgacccgcagccgcacatccagtggctaaagcacatcgaggtgaatgggagcaagattggcccagacaacctgccttatgtccagatcttgaagactgctggagttaataccaccgacaaagagatggaggtgcttcacttaagaaatgtctcctttgaggacgcaggggagtatacgtgcttggcgggtaactctatcggactctcccatcactctgcatggttgaccgttctggaagccctggaagagaggccggcagtgatgacctcgcccctgtacctggagatcatcatctattgcacaggggccttcctcatctcctgcatggtggggtcggtcatcgtctacaagatgaagagtggtaccaagaagagtgacttccacagccagatggctgtgcacaagctggccaagagcatccctctgcgcagacaggtgtctgctgactccagtgcatccatgaactctggggttcttctggttcggccatcacggctctcctccagtgggactcccatgctagcaggggtctctgagtatgagcttcccgaagaccctcgctgggagctgcctcgggacagactggtcttaggcaaacccctgggagagggctgctttgggcaggtggtgttggcagaggctatcgggctggacaaggacaaacccaaccgtgtgaccaaagtggctgtgaagatgttgaagtcggacgcaacagagaaagacttgtcagacctgatctcagaaatggagatgatgaagatgatcgggaagcataagaatatcatcaacctgctgggggcctgcacgcaggatggtcccttgtatgtcatcgtggagtatgcctccaagggcaacctgcgggagtacctgcaggcccggaggcccccagggctggaatactgctacaaccccagccacaacccagaggagcagctctcctccaaggacctggtgtcctgcgcctaccaggtggcccgaggcatggagtatctggcctccaagaagtgcatacaccgagacctggcagccaggaatgtcctggtgacagaggacaatgtgatgaagatagcagactttggcctcgcacgggacattcaccacatcgactactataaaaagacaaccaacggccgactgcctgtgaagtggatggcacccgaggcattatttgaccggatctacacccaccagagtgatgtgtggtctttcggggtgctcctgtgggagatcttcactctgggcggctccccataccccggtgtgcctgtggaggaacttttcaagctgctgaaggagggtcaccgcatggacaagcccagtaactgcaccaacgagctgtacatgatgatgcgggactgctggcatgcagtgccctcacagagacccaccttcaagcagctggtggaagacctggaccgcatcgtggccttgacctccaaccaggagtacctggacctgtccatgcccctggaccagtactcccccagctttcccgacacccggagctctacgtgctcctcaggggaggattccgtcttctctcatgagccgctgcccgaggagccctgcctgccccgacacccagcccagcttgccaatggcggactcaaacgccgctga

SEQ ID NO: 60 - protein, 820 aa, Homo sapiens
MWSWKCLLFWAVLVTATLCTARPSPTLPEQAQPWGAPVEVESFLVHPGDLLQLRCRLRDDVQSINWLRDGVQLAESNRTRITGEEVEVQDSVPADSGLYACVTSSPSGSDTTYFSVNVSDALPSSEDDDDDDDSSSEEKETDNTKPNRMPVAPYWTSPEKMEKKLHAVPAAKTVKFKCPSSGTPNPTLRWLKNGKEFKPDHRIGGYKVRYATWSIIMDSVVPSDKGNYTCIVENEYGSINHTYQLDVVERSPHRPILQAGLPANKTVALGSNVEFMCKVYSDPQPHIQWLKHIEVNGSKIGPDNLPYVQILKTAGVNTTDKEMEVLHLRNVSFEDAGEYTCLAGNSIGLSHHSAWLTVLEALEERPAVMTSPLYLEIIIYCTGAFLISCMVGSVIVYKMKSGTKKSDFHSQMAVHKLAKSIPLRRQVSADSSASMNSGVLLVRPSRLSSSGTPMLAGVSEYELPEDPRWELPRDRLVLGKPLGEGCFGQVVLAEAIGLDKDKPNRVTKVAVKMLKSDATEKDLSDLISEMEMMKMIGKHKNIINLLGACTQDGPLYVIVEYASKGNLREYLQARRPPGLEYCYNPSHNPEEQLSSKDLVSCAYQVARGMEYLASKKCIHRDLAARNVLVTEDNVMKIADFGLARDIHHIDYYKKTTNGRLPVKWMAPEALFDRIYTHQSDVWSFGVLLWEIFTLGGSPYPGVPVEELFKLLKEGHRMDKPSNCTNELYMMMRDCWHAVPSQRPTFKQLVEDLDRIVALTSNQEYLDLSMPLDQYSPSFPDTRSSTCSSGEDSVFSHEPLPEEPCLPRHPAQLANGGLKRR

SEQ ID NO: 61 - DNA, 2409 bp, Homo sapiens
atgcggctgctgctggccctgttgggggtcctgctgagtgtgcctgggcctccagtcttgtccctggaggcctctgaggaagtggagcttgagccctgcctggctcccagcctggagcagcaagagcaggagctgacagtagcccttgggcagcctgtgcgtctgtgctgtgggcgggctgagcgtggtggccactggtacaaggagggcagtcgcctggcacctgctggccgtgtacggggctggaggggccgcctagagattgccagcttcctacctgaggatgctggccgctacctctgcctggcacgaggctccatgatcgtcctgcagaatctcaccttgattacaggtgactccttgacctccagcaacgatgatgaggaccccaagtcccatagggacccctcgaataggcacagttacccccagcaagcaccctactggacacacccccagcgcatggagaagaaactgcatgcagtacctgcggggaacaccgtcaagttccgctgtccagctgcaggcaaccccacgcccaccatccgctggcttaaggatggacaggcctttcatggggagaaccgcattggaggcattcggctgcgccatcagcactggagtctcgtgatggagagcgtggtgccctcggaccgcggcacatacacctgcctggtagagaacgctgtgggcagcatccgctataactacctgctagatgtgctggagcggtccccgcaccggcccatcctgcaggccgggctcccggccaacaccacagccgtggtgggcagcgacgtggagctgctgtgcaaggtgtacagcgatgcccagccccacatccagtggctgaagcacatcgtcatcaacggcagcagcttcggagccgacggtttcccctatgtgcaagtcctaaagactgcagacatcaatagctcagaggtggaggtcctgtacctgcggaacgtgtcagccgaggacgcaggcgagtacacctgcctcgcaggcaattccatcggcctctcctaccagtctgcctggctcacggtgctgccagaggaggaccccacatggaccgcagcagcgcccgaggccaggtatacggacatcatcctgtacgcgtcgggctccctggccttggctgtgctcctgctgctggccgggctgtatcgagggcaggcgctccacggccggcacccccgcccgcccgccactgtgcagaagctctcccgcttccctctggcccgacagttctccctggagtcaggctcttccggcaagtcaagctcatccctggtacgaggcgtgcgtctctcctccagcggccccgccttgctcgccggcctcgtgagtctagatctacctctcgacccactatgggagttcccccgggacaggctggtgcttgggaagcccctaggcgagggctgctttggccaggtagtacgtgcagaggcctttggcatggaccctgcccggcctgaccaagccagcactgtggccgtcaagatgctcaaagacaacgcctctgacaaggacctggccgacctggtctcggagatggaggtgatgaagctgatcggccgacacaagaacatcatcaacctgcttggtgtctgcacccaggaagggcccctgtacgtgatcgtggagtgcgccgccaagggaaacctgcgggagttcctgcgggcccggcgccccccaggccccgacctcagccccgacggtcctcggagcagtgaggggccgctctccttcccagtcctggtctcctgcgcctaccaggtggcccgaggcatgcagtatctggagtcccggaagtgtatccaccgggacctggctgcccgcaatgtgctggtgactgaggacaatgtgatgaagattgctgactttgggctggcccgcggcgtccaccacattgactactataagaaaaccagcaacggccgcctgcctgtgaagtggatggcgcccgaggccttgtttgaccgggtgtacacacaccagagtgacgtgtggtcttttgggatcctgctatgggagatcttcaccctcgggggctccccgtatcctggcatcccggtggaggagctgttctcgctgctgcgggagggacatcggatggaccgacccccacactgccccccagagctgtacgggctgatgcgtgagtgctggcacgcagcgccctcccagaggcctaccttcaagcagctggtggaggcgctggacaaggtcctgctggccgtctctgaggagtacctcgacctccgcctgaccttcggaccctattccccctctggtggggacgccagcagcacctgctcctccagcgattctgtcttcagccacgaccccctgccattgggatccagctccttccccttcgggtctggggtgcagacatga

SEQ ID NO: 62 - protein, 802 aa, Homo sapiens
MRLLLALLGVLLSVPGPPVLSLEASEEVELEPCLAPSLEQQEQELTVALGQPVRLCCGRAERGGHWYKEGSRLAPAGRVRGWRGRLEIASFLPEDAGRYLCLARGSMIVLQNLTLITGDSLTSSNDDEDPKSHRDPSNRHSYPQQAPYWTHPQRMEKKLHAVPAGNTVKFRCPAAGNPTPTIRWLKDGQAFHGENRIGGIRLRHQHWSLVMESVVPSDRGTYTCLVENAVGSIRYNYLLDVLERSPHRPILQAGLPANTTAVVGSDVELLCKVYSDAQPHIQWLKHIVINGSSFGADGFPYVQVLKTADINSSEVEVLYLRNVSAEDAGEYTCLAGNSIGLSYQSAWLTVLPEEDPTWTAAAPEARYTDIILYASGSLALAVLLLLAGLYRGQALHGRHPRPPATVQKLSRFPLARQFSLESGSSGKSSSSLVRGVRLSSSGPALLAGLVSLDLPLDPLWEFPRDRLVLGKPLGEGCFGQVVRAEAFGMDPARPDQASTVAVKMLKDNASDKDLADLVSEMEVMKLIGRHKNIINLLGVCTQEGPLYVIVECAAKGNLREFLRARRPPGPDLSPDGPRSSEGPLSFPVLVSCAYQVARGMQYLESRKCIHRDLAARNVLVTEDNVMKIADFGLARGVHHIDYYKKTSNGRLPVKWMAPEALFDRVYTHQSDVWSFGILLWEIFTLGGSPYPGIPVEELFSLLREGHRMDRPPHCPPELYGLMRECWHAAPSQRPTFKQLVEALDKVLLAVSEEYLDLRLTFGPYSPSGGDASSTCSSSDSVFSHDPLPLGSSSFPFGSGVQT

SEQ ID NO: 63 - DNA, 2064 bp, Homo sapiens
atggtcagctactgggacaccggggtcctgctgtgcgcgctgctcagctgtctgcttctcacaggatctagttcaggttcaaaattaaaagatcctgaactgagtttaaaaggcacccagcacatcatgcaagcaggccagacactgcatctccaatgcaggggggaagcagcccataaatggtctttgcctgaaatggtgagtaaggaaagcgaaaggctgagcataactaaatctgcctgtggaagaaatggcaaacaattctgcagtactttaaccttgaacacagctcaagcaaaccacactggcttctacagctgcaaatatctagctgtacctacttcaaagaagaaggaaacagaatctgcaatctatatatttattagtgatacaggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtccaaataagcacaccacgcccagtcaaattacttagaggccatactcttgtcctcaattgtactgctaccactcccttgaacacgagagttcaaatgacctggagttaccctgatgaaaaaaataagagagcttccgtaaggcgacgaattgaccaaagcaattcccatgccaacatattctacagtgttcttactattgacaaaatgcagaacaaagacaaaggactttatacttgtcgtgtaaggagtggaccatcattcaaatctgttaacacctcagtgcatatatatgataaagcattcatcactgtgaaacatcgaaaacagcaggtgcttgaaaccgtagctggcaagcggtcttaccggctctctatgaaagtgaaggcatttccctcgccggaagttgtatggttaaaagatgggttacctgcgactgagaaatctgctcgctatttgactcgtggctactcgttaattatcaaggacgtaactgaagaggatgcagggaattatacaatcttgctgagcataaaacagtcaaatgtgtttaaaaacctcactgccactctaattgtcaatgtgaaaccccagatttacgaaaaggccgtgtcatcgtttccagacccggctctctacccactgggcagcagacaaatcctgacttgtaccgcatatggtatccctcaacctacaatcaagtggttctggcacccctgtaaccataatcattccgaagcaaggtgtgacttttgttccaataatgaagagtcctttatcctggatgctgacagcaacatgggaaacagaattgagagcatcactcagcgcatggcaataatagaaggaaagaataagatggctagcaccttggttgtggctgactctagaatttctggaatctacatttgcatagcttccaataaagttgggactgtgggaagaaacataagcttttatatcacagatgtgccaaatgggtttcatgttaacttggaaaaaatgccgacggaaggagaggacctgaaactgtcttgcacagttaacaagttcttatacagagacgttacttggattttactgcggacagttaataacagaacaatgcactacagtattagcaagcaaaaaatggccatcactaaggagcactccatcactcttaatcttaccatcatgaatgtttccctgcaagattcaggcacctatgcctgcagagccaggaatgtatacacaggggaagaaatcctccagaagaaagaaattacaatcagaggtgagcactgcaacaaaaaggctgttttctctcggatctccaaatttaaaagcacaaggaatgattgtaccacacaaagtaatgtaaaacattaa

SEQ ID NO: 64 - protein, 687 aa, Homo sapiens
MVSYWDTGVLLCALLSCLLLTGSSSGSKLKDPELSLKGTQHIMQAGQTLHLQCRGEAAHKWSLPEMVSKESERLSITKSACGRNGKQFCSTLTLNTAQANHTGFYSCKYLAVPTSKKKETESAIYIFISDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKAFITVKHRKQQVLETVAGKRSYRLSMKVKAFPSPEVVWLKDGLPATEKSARYLTRGYSLIIKDVTEEDAGNYTILLSIKQSNVFKNLTATLIVNVKPQIYEKAVSSFPDPALYPLGSRQILTCTAYGIPQPTIKWFWHPCNHNHSEARCDFCSNNEESFILDADSNMGNRIESITQRMAIIEGKNKMASTLVVADSRISGIYICIASNKVGTVGRNISFYITDVPNGFHVNLEKMPTEGEDLKLSCTVNKFLYRDVTWILLRTVNNRTMHYSISKQKMAITKEHSITLNLTIMNVSLQDSGTYACRARNVYTGEEILQKKEITIRGEHCNKKAVFSRISKFKSTRNDCTTQSNVKH

(19) United States
(12) Patent Application Publication
(10) Pub. No.: US 2014/0148483 A1
(43) Pub. Date: May 29, 2014

(54) Method For Predicting Effectiveness Of Angiogenesis Inhibitor

(71) Applicant: EISAI R&D MANAGEMENT CO., LTD., Tokyo (JP)

(72) Inventors: Taro Semba, Tsukuba-shi (JP); Yusuke Narita, Tsukuba-shi (JP); Yukinori Minoshima, Tsukuba-shi (JP); Atsumi Yamaguchi, Tsukuba-shl (JP); Yusuke Adachi, Tsukuba-shi (JP); Kazuhiko Yamada, Tsukuba-shi (JP); Junji Matsui, Tsukuba-shi (JP); Tadashi Kadowaki, Tsukuba-shi (JP); Kentaro Takahashi, Tsukuba-shi (JP); Yasuhiro Funahashi, Tsukuba-shi (JP)

(73) Assignee: EISAI R&D MANAGEMENT CO., LTD., Tokyo (JP)

(21) Appl. No.: 14/117,276

(22) PCT Filed: May 16, 2012
(86) PCT No.: PCT/JP2012/062509

§ 371 Date: Feb. 4, 2014

(30) Foreign Application Priority Data
May 17, 2011 (JP) 2011-110884

(51) Int. Cl. C12Q 1/68; A61K 31/47

(52) U.S. Cl. CPC C12Q 1/6886; A61K 31/47
USPC 514/312; 435/6.11; 546/153

(57) ABSTRACT

The purpose of the present invention is to provide a method for predicting the effectiveness of an angiogenesis inhibitor in a subject suffering from a tumor. Provided is a method comprising a step of testing for the presence or absence of an a mutation or loss of expression of B-Raf and PTEN in a sample of tumor tissue from the subject. By using the presence or absence of or a mutation or loss of expression of B-Raf and PTEN as an indicator, this method enables the antitumor effectiveness of the angiogenesis inhibitor to be predicted without administering the angiogenesis inhibitor to the subject.

### TECHNICAL FIELD

The present invention relates to a novel method for predicting the responsiveness of a subject suffering from a cancer to an angiogenesis inhibitor.

### BACKGROUND ART

Many kinase inhibitors have been developed as anticancer agents. Particularly, a group of substances having an inhibitory activity against a receptor tyrosine kinase such as Vascular Endothelial Growth factor (hereinafter also referred to as “VEGF”) receptor have characteristics of inhibiting angiogenesis associated with growth of cancer and draw attention as a new generation of anticancer agents.

However, an anticancer agent effective for all types of cancer has not yet been approved. Particularly, advanced malignant melanoma is highly metastatic and its prognosis is extremely poor. Due to this, it is difficult to develop an anticancer agent for malignant melanoma.

In the meantime, therapy with an anticancer agent generally entails side effects such as severe nausea and general malaise. Thus, administration of an anticancer agent to a subject, on which the agent is not expected to exert a therapeutic effect, should be avoided. Therefore, it has been desired to develop a biomarker by which a therapeutic effect on a subject can be predicted before an anticancer agent is administered in order to avoid administration of an ineffective medicinal drug and reduce side effects.

Incidentally, 4-(3-chloro-4-(cyclopropylaminocarbonyl)aminophenoxy)-7-methoxy-6-quinolinecarboxamide has been used as a multi-kinase inhibitor having an inhibitory activity against receptor tyrosine kinases such as VEGF receptor, Fibroblast Growth Factor (hereinafter also referred to as “FGF”) receptor, Platelet-Derived Growth Factor (hereinafter also referred to as “PDGF”) receptor, RET kinase and KIT kinase, and exhibits an excellent angiogenesis inhibition effect and an anti-growth effect (Patent Literature 1; Patent Literature 2; Non Patent Literature 1).

Furthermore, B-Raf, a kind of serine/threonine kinase, is known to serve as a cell-proliferation signal, if activated, to activate the MAP kinase pathway important as a cell-proliferation signal pathway. In addition, B-Raf has been reported to activate various types of cancer due to its mutation (Non Patent Literature 2).

Furthermore, a cancer repressor gene, PTEN (phosphatase and tensin homolog deleted on chromosome 10), encodes a lipid phosphatase which mainly utilizes PIP3 as a substrate and negatively controls the signal. PTEN has a function of inhibiting activation of Akt kinase, thereby inducing apoptosis to suppress cell-proliferation; however, a mutation and loss of expression of PTEN are known to induce excessive activation of Akt kinase, causing growth of cancer (Non Patent Literature 3).

Nevertheless, no reports have been made on association of the presence or absence of a mutation of B-Raf and the presence or absence of a mutation or loss of expression of PTEN with the anti-tumor effect of an angiogenesis inhibitor.

### CITATION LIST

### Patent Literature

Patent Literature 1: WO02/032872
Patent Literature 2: WO2007/136103

### Non Patent Literature

Non Patent Literature 1: Matsui et at, Clinical Cancer Research, 2008, 14 (17), p. 5459-5465.
Non Patent Literature 2: Davies et al., Nature, 2002, 417, p. 949-954.
Non Patent Literature 3: Besson et al., European Journal of Biochemistry, 1999, 263, p. 605-611.

### SUMMARY OF INVENTION

### Technical Problem

The present invention was made in the aforementioned circumstances. A problem to be solved by the invention is finding a method of predicting the responsiveness of a subject suffering from a cancer to an angiogenesis inhibitor, particularly, to a VEGF receptor inhibitor, an FGF receptor inhibitor, a RET kinase inhibitor or a KIT kinase inhibitor.

Another problem to be solved by the invention is selecting a subject suffering from a cancer by the above prediction method and treating the subject by administering an angiogenesis inhibitor.

### Solution to Problem

The present inventors made a great effort to solve the aforementioned problems and surprisingly found that simultaneous occurrence of a mutation of B-Raf and a mutation or loss of expression of PTEN correlates with the responsiveness of cancer cells to an angiogenesis inhibitor.

More specifically, the present inventors investigated the responsiveness of melanoma cells to an angiogenesis inhibitor. As a result, the present inventors elucidated that the case where (a1) B-Raf and PTEN are wild type or (a2) B-Raf and PTEN have a mutation or loss of expression exhibits high responsiveness to an angiogenesis inhibitor.

Additionally, the present inventors found that the presence or absence of a mutation or loss of expression in B-Raf and PTEN in melanoma cells correlates with the expression levels of angiopoietin-1 (ANG1) and angiopoietin-2 (ANG2). To describe it more specifically, it was elucidated that, in a case where (b1) the expression levels of ANG1 and ANG2 in a sample are low compared to a control value, (b2) the expression level of ANG2 in a sample is high compared to a control value or (63) the ratio of expression levels of ANG1 and ANG2 is low compared to a control value, the responsiveness of a subject suffering from a tumor to an angiogenesis inhibitor is high.

Accordingly, by use of the presence or absence of a mutation or loss of expression in B-Raf and PTEN, expression levels of ANG1 and ANG2 or the ratio of expression levels of ANG1 and ANG2 in a sample derived from a subject, as an indicator, the responsiveness of the subject to an angiogenesis inhibitor can be predicted without administration of an angiogenesis inhibitor to the subject.

In addition, the present inventors found that the anti-tumor effect pattern of an angiogenesis inhibitor, which is fluctuated with the presence or absence of a mutation or loss of expression in B-Raf and PTEN in melanoma cells, also correlates with the expression levels of SHC1, IL6, CXCR4, COL4A3, NRP2, MEIS1, ARHGAP22, SCG2, FGF9, PML, FGFR3, FGFR2, FGFR1, FGFR4 and VEGFR1. To describe it more specifically, they elucidated that the responsiveness of a subject suffering from a tumor to an angiogenesis inhibitor is high in a case where (c1) the expression level of SHC1 is low compared to a control value, (c2) the expression level of IL6 is high compared to a control value, (c3) the expression level of CXCR4 is high compared to a control value, (c4) the expression level of COL4A3 is high compared to a control value, (c5) the expression level of NRP2 is low compared to a control value, (c6) the expression level of MEIS1 is high compared to a control value, (c7) the expression level of ARHGAP22 is low compared to the a control value, (c8) the expression level of SCG2 is low compared to a control value, (c9) the expression level of FGF9 is high compared to a control value, (c10) the expression level of PML is low compared to a control value, (c11) the expression level of FGFR3 is high compared to a control value, (c12) the expression level of FGFR2 is high compared to a control value, (c13) the expression level of FGFR1 is high compared to a control value, (c14) the expression level of FGFR4 is high compared to a control value, or (c15) the expression level of VEGFR1 is high compared to a control value.

Specifically, the present invention relates to the following.

(1) A method for predicting the responsiveness of a subject suffering from a tumor to an angiogenesis inhibitor, comprising

(a) detecting the presence or absence of a mutation or loss of expression of B-Raf and the presence or absence of a mutation or loss of expression of PTEN in a sample derived from a tumor tissue of the subject, wherein in the detection step, a case where

(a1) B-Raf is wild type and PTEN is wild type, or

(a2) B-Raf has at least one mutation selected from Table 1 or loss of expression and PTEN has at least one mutation selected from Table 2 or loss of expression is indicative of the high responsiveness of the subject to the angiogenesis inhibitor.

(2) The method according to (1), wherein, in the detection step (a), a case where B-Raf is wild type and PTEN is wild type is indicative of the high responsiveness of the subject to the angiogenesis inhibitor.

(3) The method according to (1), wherein, in the detection step (a), a case where B-Raf has at least one mutation selected from Table 1 or loss of expression and PTEN has at least one mutation selected from Table 2 or loss of expression is indicative of the high responsiveness of the subject to the angiogenesis inhibitor.

(4) The method according to (1) or (3), wherein the mutation of B-Raf is a V600E mutation in an amino acid sequence or a mutation in a nucleotide sequence corresponding to the mutation.

(5) The method according to (1) or (3), wherein the mutation of PTEN is at least one mutation in a nucleotide sequence selected from the group consisting of A499G, T202C and T335A or at least one mutation in an amino acid sequence selected from the group consisting of T167A, Y68H and L112Q.

(6) The method according to any one of (1) to (5), wherein the angiogenesis inhibitor is 4-(3-chloro-4-(cyclopropylaminocarbonyl)aminophenoxy)-7-methoxy-6-quinolinecarboxamide or a pharmacologically acceptable salt thereof.

(7) The method according to (6), wherein the angiogenesis inhibitor is a mesylate salt of 4-(3-chloro-4-(cyclopropylaminocarbonyl)aminophenoxy)-7-methoxy-6-quinolinecarboxamide.

(8) The method according to any one of (1) to (7), wherein the tumor is a tumor having a V600E mutation in B-Raf.

(9) The method according to any one of (1) to (8), wherein the tumor is melanoma, thyroid cancer, colorectal cancer, ovarian cancer, liver cancer, lung cancer, endometrial cancer or glioma.

(10) The method according to any one of (1) to (9), wherein, in the step (a), the high responsiveness of the subject to the angiogenesis inhibitor is predicted; and the method further comprises a step (b) of quantifying expression levels of ANG1 and ANG2 in the sample derived from the tumor tissue of the subject, wherein, in the quantification step, a case where

(b1) the expression level of ANG1 is low compared to a control value

(b2) the expression level of ANG2 is high compared to a control value, or

(b3) the ratio of the expression levels of ANG1 and ANG2 is low compared to a control value

is indicative of the high responsiveness of the subject to the angiogenesis inhibitor.

(11) The method according to any one of (1) to (9), wherein, in the step (a), the high responsiveness of the subject to the angiogenesis inhibitor is predicted; and the method further comprises a step (c) of quantifying an expression level of at least one selected from the group consisting of SHC1, IL6, CXCR4, COL4A3, NRP2, MEIS1, ARHGAP22, SCG2, FGF9, PML, FGFR3, FGFR2, FGFR1, FGFR4 and VEGFR1 in the sample derived from the tumor tissue of the subject, wherein, in the quantification step, a case where

(c1) the expression level of SHC1 is low compared to a control value,

(c2) the expression level of NRP2 is low compared to a control value,

(c3) the expression level of ARHGAP22 is low compared to a control value,

(c4) the expression level of SCG2 is low compared to a control value,

(c5) the expression level of PML, is low compared to a control value,

(c6) the expression level of IL6 is high compared to a control value,

(c7) the expression level of CXCR4 is high compared to a control value,

(c8) the expression level of COL4A3 is high compared to a control value,

(c9) the expression level of MEIS1 is high compared to a control value,

(c10) the expression level of FGF9 is high compared to a control value,

(c11) the expression level of FGFR3 is high compared to a control value,

(c12) the expression level of FGFR2 is high compared to a control value,

(c13) the expression level of FGFR1 is high compared to a control value,

(c14) the expression level of FGFR4 is high compared to a control value, or

(c15) the expression level of VEGFR1 is high compared to a control value is indicative of the high responsiveness of the subject to the angiogenesis inhibitor.

(12) A method for predicting the responsiveness of a subject suffering from a tumor to an angiogenesis inhibitor, comprising

(b) quantifying expression levels of ANG1 and ANG2 in a sample derived from a tumor tissue of the subject, wherein, in the quantification step, a case where

(b1) the expression level of ANG1 is low compared to a control value

(b2) the expression level of ANG2 is high compared to a control value, or

(b3) the ratio of expression level of ANG1 and ANG2 is low compared to a control value

is indicative of the high responsiveness of the subject to the angiogenesis inhibitor.

(13) The method according to (12), wherein, in the step (b), the high responsiveness of the subject to the angiogenesis inhibitor is predicted, and the method further comprises a step (c) of quantifying an expression level of at least one selected from the group consisting of SHC1, IL6, CXCR4, COL4A3, NRP2, MEIS1, ARHGAP22, SCG2, FGF9, PML, FGFR3, FGFR2, FGFR1, FGFR4 and VEGFR1 in the sample derived from the tumor tissue of the subject, wherein, in the quantification step, a case where

(c1) the expression level of SHC1 is low compared to a control value,

(c2) the expression level of NRP2 is low compared to a control value,

(c3) the expression level of ARHGAP22 is low compared to a control value,

(c4) the expression level of SCG2 is low compared to a control value,

(c5) the expression level of PML is low compared to a control value,

(c6) the expression level of IL6 is high compared to a control value,

(c7) the expression level of CXCR4 is high compared to a control value,

(c8) the expression level of COL4A3 is high compared to a control value,

(c9) the expression level of MEIS1 is high compared to a control value,

(c10) the expression level of FGF9 is high compared to a control value,

(c11) the expression level of FGFR3 is high compared to a control value,

(c12) the expression level of FGFR2 is high compared to a control value,

(c13) the expression level of FGFR1 is high compared to a control value,

(c14) the expression level of FGFR4 is high compared to a control value, or

(c15) the expression level of VEGFR1 is high compared to a control value is indicative of the high responsiveness of the subject to the angiogenesis inhibitor.

(14) The method according to any one of (1) to (13), wherein the step (a) to (c) comprise a step of bringing the sample derived from the tumor tissue of the subject into contort with probes of B-Raf and PTEN. Particularly, the probes are preferably a nucleic acid probe, a specific antibody or a combination thereof.

(15) A method for treating a subject suffering from a tumor by administration of an angiogenesis inhibitor, wherein the subject has been predicted to be highly responsive to the angiogenesis inhibitor by the method according to any one of (1) to (14).

(16) The method according to (15), wherein the angiogenesis inhibitor is 4-(3-chloro-4-(cyclopropylaminocarbonyl)aminophenoxy)-7-methoxy-6-quinolinecarboxamide or a pharmacologically acceptable salt thereof.

(17) Use of an angiogenesis inhibitor in the manufacture of a medicament to be used for administration to a subject suffering from a tumor, wherein the subject has been predicted to be highly responsive to the angiogenesis inhibitor by the method according to any one of (1) to (14).

(18) The use according to (17), wherein the angiogenesis inhibitor is 4-(3-chloro-4-(cyclopropylaminocarbonyl)aminophenoxy)-7-methoxy-6-quinolinecarboxamide or a pharmacologically acceptable salt thereof.

(19) A pharmaceutical composition comprising an angiogenesis inhibitor for treating a subject suffering from a tumor, wherein the subject has been predicted to be highly responsive to the angiogenesis inhibitor by the method according to any one of (1) to (14).

(20) The pharmaceutical composition according to (19), wherein the angiogenesis inhibitor is 4-(3-chloro-4-(cyclopropylaminocarbonyl)aminophenoxy)-7-methoxy-6-quinolinecarboxamide or a pharmacologically acceptable salt thereof.

(21) An angiogenesis inhibitor for treating a subject suffering from a tumor, wherein the subject has been predicted to be highly responsive to the angiogenesis inhibitor by the method according to any one of (1) to (14) by a doctor or another medical practitioner who administer the therapy.

As the angiogenesis inhibitor, 4-(3-chloro-4-(cyclopropylaminocarbonyl)aminophenoxy)-7-methoxy-6-quinolinecarboxamide or a pharmacologically acceptable salt thereof and is preferable and a mesylate salt of 4-(3-chloro-4-(cyclopropylaminocarbonyl)aminophenoxy)-7-methoxy-6-quinolinecarboxamide is particularly preferable.

(22) A kit for predicting the responsiveness of a subject suffering from a tumor to an angiogenesis inhibitor, comprising probes of B-Raf and PTEN or probes of ANG1 and ANG2, wherein the responsiveness of the subject suffering from the tumor to the angiogenesis inhibitor is predicted by the method according to any one of (1) to (14).

As the angiogenesis inhibitor, 4-(3-chloro-4-(cyclopropylaminocarbonyl)aminophenoxy)-7-methoxy-6-quinolinecarboxamide or a pharmacologically acceptable salt thereof and is preferable and a mesylate salt of 4-(3-chloro-4-(cyclopropylaminocarbonyl)aminophenoxy)-7-methoxy-6-quinolinecarboxamide is particularly preferable.

### Advantageous Effects of Invention

The present invention enables to predict the responsiveness of a subject suffering from a cancer to an angiogenesis inhibitor, and in particular, predict the responsiveness to a VEGF receptor inhibitor, an FGF receptor inhibitor, a RET kinase inhibitor or a KIT kinase inhibitor.

As a result, whether administration of an angiogenesis inhibitor to a subject suffering from a cancer is effective or not is determined, and thereafter, the angiogenesis inhibitor can be administered to the subject. Therefore, cancer patients, for which administration of the angiogenesis inhibitor is effective, are selected, and then, the angiogenesis inhibitor can be administered, In this manner, cancer can be treated while reducing the risk of a side effect.

### BRIEF DESCRIPTION OF DRAWINGS

FIG. 1 is a graph showing the anti-tumor effect of E7080 on each of melanoma cells, which are classified into groups based on the presence or absence of a mutation or loss of expression in BRAF and PTEN. In BRAF, “+” indicates the presence of a mutation or loss of expression; whereas “−” indicates the absence of a mutation or loss of expression. In PTEN, “+” indicates the presence of a mutation or a loss of expression; whereas “−” indicates the absence of a mutation or loss of expression.

FIG. 2 is a graph showing the ratio of pericyte-covered blood vessels in each of the tumors of melanoma cells, which are classified into groups based on the presence or absence of a mutation or loss of expression in BRAF and PTEN. In BRAF, “+” indicates the presence of a mutation or loss of expression; whereas “−” indicates the absence of a mutation or loss of expression. In PTEN, “+” indicates the presence of a mutation or loss of expression; whereas “−” indicates the absence of a mutation or loss of expression.

FIG. 3 is a graph showing the correlation between the anti-tumor effect of E7080 on melanoma cells and the ratio of pericyte-covered blood vessels in the tumor.

FIG. 4 is a graph showing (a) the expression level of ANG1 protein and (b) the expression level of ANG1 mRNA in melanoma cells, which are classified into groups based on the presence or absence of a V600E mutation in BRAF. The symbol “+” indicates the presence of a mutation or loss of expression; whereas “−” indicates the absence of a mutation or loss of expression.

FIG. 5 is a graph showing (a) the expression level of ANG2 protein and (b) the expression level of ANG2 mRNA, in melanoma cells, which are classified into groups based on the presence or absence of a mutation or loss of expression in PTEN. The symbol “+” indicates the presence of a mutation or loss of expression; whereas “−” indicates the absence of a mutation or loss of expression.

FIG. 6 is a graph showing the ratio of expression levels of ANG1 and ANG2 in melanoma cells, which are classified into groups based on the presence or absence of a mutation or loss of expression in BRAF and PTEN. In BRAF, “+” indicates the presence of a mutation or loss of expression; whereas “−” indicates the absence of a mutation or loss of expression. In PTEN, “+” indicates the presence of a mutation or loss of expression; whereas “−” indicates the absence of a mutation or loss of expression.

FIG. 7 is a graph showing the ratio of expression levels of FGFR2 and FGFR3 in melanoma cells, which are classified into groups based on the presence or absence of a mutation or loss of expression in BRAF and PTEN. The term “BRAF wt/PTEN wt” indicates a case where BRAF and PTEN are wild type; “BRAF mu/PTEN mu” indicates a case where BRAF and PTEN have a mutation or loss of expression; and “BRAF mu/PTEN wt” indicates a case where BRAF has a mutation or loss of expression and PTEN is wild type.

### DESCRIPTION OF EMBODIMENTS

Embodiments of the present invention will be described below. The following embodiments are examples for explaining the present invention and should not be construed as limiting the present invention. The present invention can be carried out in various ways as long as they do not depart from the spirit of the invention.

Note that literatures and publications of patent applications laid-open, patent gazettes and other patent literatures are incorporated in the specification as references.

The present invention relates to a method for predicting the responsiveness of a subject to an angiogenesis inhibitor.

The method of the present invention comprises a step of detecting the presence or absence of a mutation or loss of expression of B-Raf and the presence or absence of a mutation or loss of expression of PTEN in a sample derived from a tumor tissue of a subject.

In the detection step, the following case of (a1) or (a2) serves as an indicator that the responsiveness of the subject to an angiogenesis inhibitor is high.

(a1) B-Raf is wild type and PTEN is wild type.

(a2) B-Raf has at least one mutation selected from Table 1 or loss of expression mutation and PTEN has at least one mutation selected from Table 2 or loss of expression mutation.

Furthermore, the method of the present invention comprises a step of quantifying expression levels of ANG1 and ANG2 in a sample derived from a tumor tissue of a subject. In the quantification step, these quantification results in the following (b1), (b2) or (b3) serve as an indicator that the responsiveness of the subject to an angiogenesis inhibitor is high.

(b1) the expression level of ANG1 is low compared to a control value.

(b2) the expression level of ANG2 is high compared to a control value.

(b3) the ratio of the expression levels of ANG1 and ANG2 is low compared to a control value.

Furthermore, the present invention comprises a step of quantifying the expression level of at least one selected from the group consisting of SHC1, IL6, CXCR4, COL4A3, NRP2, MEIS1, ARHGAP22, SCG2, FGF9, PML, FGFR3, FGFR2, FGFR1, FGFR4 and VEGFR1 in a sample derived from a tumor tissue of the subject. In the quantification step, if a case corresponds to the following (c1) to (c15), these quantification results serve as an indicator that the responsiveness of the subject to an angiogenesis inhibitor is high.

(c1) the expression level of SHC1 is low compared to a control value.

(c2) the expression level of NRP2 is low compared to a control value.

(c3) the expression level of ARHGAP22 is low compared to a control value.

(c4) the expression level of SCG2 is low compared to a control value.

(c5) the expression level of PML is low compared to a control value.

(c6) the expression level of IL6 is high compared to a control value.

(c7) the expression level of CXCR4 is high compared to a control value.

(c8) the expression level of COL4A3 is high compared to a control value.

(c9) the expression level of MEIS1 is high compared to a control value.

(c10) the expression level of FGF9 is high compared to a control value.

(c11) the expression level of FGFR3 is high compared to a control value.

(c12) the expression level of FGFR2 is high compared to a control value.

(c13) the expression level of FGFR1 is high compared to a control value.

(c14) the expression level of FGFR4 is high compared to a control value.

(c15) the expression level of VEGFR1 is high compared to a control value.

Furthermore, the present invention comprises a step of detecting the presence or absence of a mutation or loss of expression in B-Raf and PTEN and the expression levels of FGFR3 or FGFR2 in a sample derived from a tumor tissue of the subject. In the detection step, the following case of (d1) or (d2) serves as an indicator that the responsiveness of the subject to an angiogenesis inhibitor is high.

(d1) B-Raf and PTEN each are wild type and FGFR3 or FGFR2 is expressed.

(d2) B-Raf has at least one mutation selected from Table 1 or loss of expression, and PTEN has at least one mutation selected from Table 2 or loss of expression, and FGFR3 or FGFR2 is expressed.

Furthermore, the method of the present invention relates to a method for predicting the responsiveness to an angiogenesis inhibitor by use of the above indicators.

More specifically, the method of the present invention comprises a step of detecting the presence or absence of a mutation or loss of expression mutation of B-Raf and PTEN; expression levels of ANG1 and ANG2; or the ratio of expression levels of ANG1 and ANG2, and associating these detection results used as an indicator with the responsiveness to an angiogenesis inhibitor. The method of the present invention also comprises a step of quantifying the expression level of SHC1, IL6, CXCR4, COL4A3, NRP2, MEIS1, ARHGAP22, SCG2, FGF9, PML, FGFR3, FGFR2, FGFR1, FGFR4 or VEGFR1 and associating these detection results used as an indicator with the responsiveness to an angiogenesis inhibitor.

In the present invention, the detection step may comprise a step of determining expression level or the ratio of expression levels or a step of analyzing the determination results obtained; and the quantification step may comprise a step of determining expression level or the ratio of expression levels, or a step of analyzing the determination results obtained.

The above detection results and quantification results obtained by the method of the present invention are provided as information for determining whether or not the subject is highly responsible to an angiogenesis inhibitor. These pieces of information are mainly used by medical practitioner.

When it is determined that the responsiveness to an angiogenesis inhibitor is high by the method of the present invention, it can be expected that the angiogenesis inhibitor effectively works (has an anti-tumor effect). Thus, the method of the present invention can be used as an indicator for a cancer therapy.

The angiogenesis inhibitor, which is a target of the method of the present invention, is a substance having an angiogenesis inhibitory activity. Examples of the angiogenesis inhibitor include a VEGF receptor inhibitor, an FGF receptor inhibitor, a PDGF receptor inhibitor, a RET kinase inhibitor, a KIT kinase inhibitor, an epithelial growth factor (EGF) inhibitor, an integrin inhibitor, a matrix metalloprotease inhibitor and an endogenous inhibitor.

“VEGF receptor” belongs to a group of receptor tyrosine kinases. In the present invention, VEGFR-1 (also referred to as Flt-1), VEGFR-2 (also referred to as KDR/Flk-1) and VEGFR-3 (also referred to as Flt-4) are collectively referred to as a VEGF receptor. Furthermore, a substance, which has a homology with the amino acid sequence of any one of VEGFR-1, VEGFR-2 and VEGFR-3 and has a VEGF receptor activity (including a receptor whose function presently remains unknown but will be classified in the same family in future), is also included in VEGF receptor. The VEGF receptor activity can be determined by detecting phosphorylation of the receptor by means of ELISA or Western blotting using a specific antibody.

In the present invention, the “VEGF receptor inhibitor” refers to an inhibitor having an inhibitory activity against VEGF receptor. The VEGF receptor inhibitor may have inhibitory activities against other receptor tyrosine kinases and other biological molecules as long as it has an inhibitory activity against VEGF receptor.

“FGF receptor” belongs to a group of receptor tyrosine kinases. In the present invention, FGFR1, FGFR2, FGFR3, FGFR4 and FGFR5 are collectively referred to as an FGF receptor. Furthermore, a substance, which has a homology with the amino acid sequence of any one of FGFR1, FGFR2, FGFR3, FGFR4 and FGFR5 and has an FGF receptor activity (including a receptor whose function presently remains unknown but will be classified in the same family in future), is also included in the FGF receptor. The FGF receptor activity can be determined by detecting phosphorylation of the receptor by means of ELISA or Western blotting.

In the present invention, the “FGF′ receptor inhibitor” refers to an inhibitor having an inhibitory activity against an FGF receptor. The FGF receptor inhibitor may have inhibitory activities against other receptor tyrosine kinases and other biological molecules as long as it has an inhibitory activity against an FGF receptor.

“PDGF receptor” belongs to a group of receptor tyrosine kinases. In the present invention, PDGFR-α and PDGFR-β are collectively referred to as PDGF receptor. Furthermore, a substance, which has a homology with the amino acid sequence of any one of PDGFR-α and PDGFR-β and has a PDGF receptor activity (including a receptor whose function presently remains unknown but will be classified in the same family in future), is also included in PDGF receptor. The PDGF receptor activity can be determined by detecting phosphorylation activity of the receptor by means of ELISA or Western blotting.

In the present invention, the “PDGF receptor inhibitor” refers to an inhibitor having an inhibitory activity against PDGF receptor. The PDGF receptor inhibitor may have inhibitory activities against other receptor tyrosine kinases and other biological molecules as long as it has an inhibitory activity against PDGF receptor.

“RET kinase”, which belongs to a group of receptor tyrosine kinases, is a functional receptor for a ligand of Glia cell-line Derived Neurotropic Factor (GDNF) family. In the present invention, furthermore, a substance, which has a homology with the amino acid sequence of RET kinase and has a RET kinase activity (including a receptor whose function presently remains unknown but will be classified in the same family in future), is also included in RET kinase. The RET kinase activity can be determined by detecting phosphorylation activity of the receptor by means of ELISA or Western blotting.

In the present invention, the “RET kinase inhibitor” refers to an inhibitor having an inhibitory activity against RET kinase. The RET kinase inhibitor may have inhibitory activities against other receptor tyrosine kinases and other biological molecules as long as it has an inhibitory activity against RET kinase.

“KIT kinase”, which is also referred to as c-Kit or an SCF receptor, belongs to a group of receptor tyrosine kinases. In the present invention, furthermore, a substance, which has a homology with the amino acid sequence of the KIT kinase and has a KIT kinase activity (including a substance whose function presently remains unknown but will be classified in the same family in future), is also included in KIT kinase.

In the present invention, the “KIT kinase inhibitor” refers to an inhibitor having an inhibitory activity against KIT kinase. The KIT kinase inhibitor may have inhibitory activities against other receptor tyrosine kinases and other biological molecules as long as it has an inhibitory activity against KIT kinase. The KIT kinase activity can be determined by detecting phosphorylation activity of the receptor by means of ELISA or Western blotting method.

“EGF” refers to Epithelial Growth Factor and the “EGF inhibitor” refers to an inhibitor having inhibitory activity against signaling induced by binding of EGF to its receptor. The EGF inhibitor may have inhibitory activities against other biological molecules as long as it has an inhibitory activity against signaling induced by EGF.

“Integrin” is one of cell surface proteins mainly serving as a cell adhesion molecule. The structure is a heterodimer consisting of an α chain and a β chain. Up to present, 22 types of integrins consisting of different α chains and β chains in combination have found and form an integrin family. The “integrin inhibitor” refers to an inhibitor having an inhibitory activity against signaling induced by binding of integrin to its receptor. The integrin inhibitor may have inhibitory activities against other biological molecules as long as it has an inhibitory activity against signaling induced by integrin.

“Matrix metalloprotease” belongs to a group of zinc ion (Zn²⁺)-dependent proteases involved in degradation of extracellular matrix. The matrix metalloprotease is known to degrade the basal membrane around blood vessels, thereby enhancing angiogenesis. The “matrix metalloprotease inhibitor” refers to an inhibitor having an inhibitory activity against matrix metalloprotease. The matrix metalloprotease inhibitor may have inhibitory activities against other biological molecules as long as it has an inhibitory activity against matrix metalloprotease.

The “endogenous inhibitor” refers to a biological substance having an angiogenesis inhibitory activity endogenously expressed by cells and includes thrombospondin, prolactin, interferon α/β, interleukin-12, platelet factor 4, angiostatin, endostatin, or degradation products thereof.

In the present invention, the “responsiveness” to an angiogenesis inhibitor refers to a nature of cancer cells, the growth of which is suppressed by administration of an angiogenesis inhibitor, used as an indicator for sensitivity to an angiogenesis inhibitor.

The “tumor” herein is classified into a benign tumor and a malignant tumor and each is further classified into an epithelial tumor and a non-epithelial tumor. In the present invention, “cancer” is, in some cases, referred to as an epithelial malignant tumor.

The “high responsiveness” of a subject to an angiogenesis inhibitor can refer to a nature of tumor cells, the growth of which is strongly suppressed by administration of the angiogenesis inhibitor, and, for example, means that growth of tumor cells, for example, in terms of growth rate or growth yield of tumor cells, relative to a control value, is ½ or less, preferably ⅕ or less and further preferably 1/10 or less; or that the colony forming activity of tumor cells relative to a control value is ½ or less, preferably ⅕ or less and further preferably 1/10 or less.

Alternatively, in a clinical scene, the “high responsiveness” can mean that an increase of lesions is suppressed, for example, within 20% compared to a control value by administration of an angiogenesis inhibitory substance; and preferably means that the sum of the longest diameter of target lesions decreases by 30% or more compared to that before administration, and further preferably means that all target lesions disappear; however, the “high responsiveness” is not limited to these examples.

In the present invention, “a sample derived from a tumor tissue of a subject” refers to a tumor tissue taken from a subject, tumor cells dissociated from a tumor tissue such as circulating tumor cells, or DNA, RNA (for example, mRNA, miRNA, tRNA, rRNA, ncRNA, dsRNA, snRNA, snoRNA), other nucleic acids or proteins derived from tumor cells; or preparations made from these into the forms suitable for carrying out the present invention. The tumor tissue or tumor cells taken from a subject may be a body fluid or blood. Note that a person who takes samples and makes preparations may be same or different from a medical practitioner performing the steps of the present invention.

In the present invention, the “medical practitioner” refers to doctors, dentists, laboratory technicians (including experts for performing testing in testing service providers), nurses and workers of other medical institutions.

In the present invention, examples of the type of tumor, the responsiveness of which to an angiogenesis inhibitor is a target to be predicted or the type of tumor that a subject has, include, but not particularly limited to, brain tumors (including pituitary adenoma, glioma), head and neck cancer, neck cancer, chin cancer, upper jaw cancer, submaxillary gland cancer, oral cavity cancer (including tongue cancer, floor of mouth cancer, gingival cancer, buccal mucosa cancer, hard palate cancer), saliva gland cancer, sublingual gland cancer, parotoid cancer, nasal cavity cancer, paranasal cancer (including maxillary sinus cancer, frontal sinus cancer, ethmoid sinus cancer, sphenoid sinus cancer), laryngeal cancer (including supraglottic cancer, glottic cancer, subglottic cancer), esophagus cancer, lung cancer (including primitive bronchial cancer, non-small-cell lung cancer (including pulmonary adenocarcinoma, squamous cancer, large-cell lung cancer), small-cell lung cancer (including oat cell cancer (lymphoidcyte type), intermediary cell type), mixed small cell/large cell lung cancer), breast cancer, pancreatic cancer (including pancreatic ductal cancer), stomach cancer (including scirrhous stomach cancer, undifferentiated stomach cancer (including low-differentiated glandular cancer, signet ring cell cancer, mucinous carcinoma)), biliary cancer (including bile duct cancer, gallbladder cancer), small intestinal cancer or duodenal cancer, large bowel cancer (including colon cancer, rectal cancer, colorectal cancer, cecal cancer, sigmoid colon cancer, ascending colon cancer, transverse colon cancer, descending colon cancer), bladder cancer, kidney cancer (including renal cell cancer), liver cancer (including hepatocellular carcinoma, intrahepatic bile duct cancer), prostate cancer, uterine cancer (including uterine cervix cancer, uterine body cancer), ovarian cancer, thyroid cancer, pharyngeal cancer (including nasopharyngeal carcinoma, mesopharyngeal carcinoma, hypopharyngeal carcinoma), sarcoma (for example, osteosarcoma, chondrosarcoma, Kaposi sarcoma, myosarcoma, angiosarcoma, fibrosarcoma), malignant lymphoma (including Hodgkin's lymphoma, non-Hodgkin's lymphoma), leukemia (including for example, chronic myelogenous leukemia (CML), acute myelogenous leukemia (AML), chronic lymphocytic leukemia (CLL) and acute lymphoblastic leukemia (ALL), lymphoma, multiple myeloma (MM), myelodysplastic syndrome) and skin cancer (including basal cell carcinoma, prickle cell cancer, melanoma, mycosis fungoides, Sezary syndrome, solar keratosis, Bowen's disease, Paget's disease) and preferably include tumors having a V600E mutation in B-Raf. Examples of the tumors having a V600E mutation in B-Raf include melanoma, thyroid cancer, ovarian cancer, biliary tract cancer, large bowel cancer, liver cancer, pancreatic cancer, breast cancer, lung cancer, glioma, myelogenous leukemia and endometrial cancer (Schubbert et al., Nature Reviews Cancer, 2007, 7, p. 295-309). More preferably, melanoma, thyroid cancer, large bowel cancer, ovarian cancer, liver cancer, lung cancer, endometrial cancer and glioma are mentioned and further preferably, melanoma is mentioned. Whether a tumor is one having a V600E mutation in B-Raf can be checked by a detection method (described later) for a mutation or a loss of expression in B-Raf.

The subject in the present invention includes a subject suffering from at least one type of tumor selected from the aforementioned ones. As long as a subject is suffering from at least one type of tumor selected from the aforementioned ones, the subject may be suffering from other diseases.

In the present invention, “B-Raf” (v-raf murine sarcoma viral oncogene homolog B1) (also referred to as “BRAF”), which is a serine/threonine protein kinase belonging to a raf/mil family, refers to the gene (SEQ ID NO: 1) represented by the polynucleotide sequence under GenBank Accession No. NM_{—}004333.4 which is determined from its mRNA, and refers to the protein (SEQ ID NO: 2) under GenBank Accession No. NP_{—}004324.2, which is translated from the gene. The protein has a function of controlling the MAP kinase/ERKs signaling pathway.

In the present invention, “PTEN” (phosphatase and tensin homolog deleted on chromosome 10) refers to the gene (SEQ ID NO: 3) represented by the polynucleotide sequence under GenBank Accession No. NM_{—}000314.4 which is determined from its mRNA, and refers to the protein (SEQ ID NO: 4) under GenBank Accession No. NP_{—}000305.3, which is translated from the gene.

In the present invention, a “mutation” of B-Raf or PTEN refers to a variation of a single or a plurality of nucleotides in the polynucleotide sequence and/or a single or a plurality of amino acids in the amino acid sequence of B-Raf or PTEN, caused by substitution, deletion, insertion and/or addition. Therefore, if the state in which a substitution, deletion, insertion and/or addition of one or a plurality of nucleotides in the polynucleotide sequence and/or one or a plurality of amino acids in the amino acid sequence of B-Raf or PTEN is detected, it is determined that B-Raf or PTEN has a mutation.

In the present invention, a mutation of B-Raf is, e.g., a mutation of the amino acid sequence selected from the mutations shown in the following Table 1 or a mutation of the nucleotide sequence corresponding to the ma nation of the amino acid sequence.

TABLE 1
Mutation of B-Raf
Amino acid

| | | |
|---|---|---|
| D587A | G596R | R444Q |
| D587E | | R444W |
| D594E | G615E | R462I |
| D594V | | |
| D594G | | |
| E586K | I463S | S605F |
| | | S605N |
| F468C | I592M | T599_V600insTT |
| | I592V | T599I |
| F595L | K601del | V471F |
| F595S | K601E | |
| | K601N | |
| G464R | L597Q | V600A |
| G464V | L597V | V600D |
| G464E | L597S | V600E |
| | L597R | V600K |
| | | V600M |
| | | V600R |
| | | V600L |
| G466R | N581S | A145V |
| G466V | | |
| G469S | R443T | |
| G469E | | |
| G469A | | |

In Table 1, the numeric character sandwiched between alphabets indicates the position in the amino acid sequence (SEQ ID NO: 2) of B-Raf; and the alphabet before the numeric character is an amino acid of wild type and the alphabet after the numeric character is an amino acid of mutant.

To explain more specifically, a mutation D587A in the amino acid sequence means that, in the amino acid sequence (SEQ ID NO: 2) encoded by the B-Raf gene (SEQ ID NO: 1), aspartic acid at position 587 is mutated to alanine or refers to a mutation of the polynucleotide sequence corresponding to the mutation of the amino acid sequence.

A mutation V600E in the amino acid sequence means that, in the amino acid sequence (SEQ ID NO: 2) encoded by the B-Raf gene (SEQ ID NO: 1), valine at position 600 is mutated to glutamic acid, or refers to a mutation of the corresponding polynucleotide sequence; for example, in “gtg” corresponding to the positions from 1798 to 1800 of the nucleotide sequence, the nucleotide at position 1799 is mutated from thymine to adenine.

A mutation K601del in the amino acid sequence means that in the amino acid sequence (SEQ ID NO: 2) encoded by the B-Raf gene (SEQ ID NO: 1), lysine at position 601 is deleted or refers to a mutation of the corresponding polynucleotide sequence.

A mutation T599_V600insTT in the amino acid sequence means that, in the amino acid sequence (SEQ ID NO: 2) encoded by the B-Raf gene (SEQ ED NO: 1), two threonine residues are inserted between 599th threonine and 600th valine or refers to a mutation of the corresponding nucleotide sequence.

In the present invention, the “loss of expression” or “loss of expression mutation” of B-Raf means that B-Raf protein is not expressed by deletion of the B-Raf gene or a mutation of a polynucleotide sequence of the B-Raf gene (including an intron). Therefore, if the detection level of polynucleotide sequence and/or amino acid sequence of B-Raf in a sample derived from a tumor tissue of a subject is statistically significantly low compared to a control value or less than a previously determined cutoff value, or if B-Raf is a detection limit or less, it is determined that the expression of B-Raf is lost.

In the present invention, the “wild type” of B-Raf refers to the state where if the presence or absence of at least one of the mutation sites shown in Table 1 is checked, neither mutation nor loss of expression is detected. Furthermore, B-Raf being “wild type” is referred also to B-Raf “under normal”.

In the present invention, the mutation of PTEN is one selected from those shown in the following Table 2.

TABLE 2
Mutation of PTEN

| Nucleotide | Amino acid |
|---|---|
| T170G | L57W |
| T202C | Y68H |
| T228G | Y76stop |
| T335A | L112Q |
| C367T | H123Y |
| T370A | C124S |
| G371C | |
| G385C | G129R |
| G385A | |
| G493A | G165R |
| A499G | T167A |
| A499C | T167P |

In Table 2, the numeric character sandwiched between alphabets indicates the position of the polynucleotide sequence (SEQ ID NO: 3) or the amino acid sequence (SEQ ID NO: 4) of PTEN; the alphabet before the numeric character is the nucleotide sequence or amino acid sequence of wild type; and the alphabet after the numeric character is the nucleotide sequence or amino acid sequence of mutant. To explain more specifically, the nucleotide mutation T170G means that, the nucleotide of position 170 in the protein coding region (SEQ ID NO: 3) of PTEN gene is mutated from thymine to guanine. The amino acid mutation L57W means that the 57th leucine in the corresponding amino acid sequence (SEQ ID NO: 4) of the protein is mutated to tryptophan. Y76stop means that the 76th tyrosine codon of the amino acid sequence of PTEN varies to a stop codon, by which translation is terminated.

In the present invention, the “loss of expression” or “loss of expression mutation” of PTEN means the state where PTEN protein is not expressed by deletion of the PTEN gene or a mutation of a polynucleotide sequence of the PTEN gene (including an intron). Therefore, if the detection level of the polynucleotide sequence and/or amino acid sequence of PTEN in a sample derived from a tumor tissue of a subject is statistically significantly low compared to a control value or less than a previously determined cutoff value, or if PTEN is a detection limit or less, it is determined that the expression of PTEN is lost.

In the present invention, the “wild type” of PTEN refers to the state where if the presence or absence of at least one of the mutation sites shown in Table 2 is checked, neither mutation nor loss of expression is detected. Furthermore, PTEN being “wild type” is referred also to PTEN “under normal”.

In the present invention, “ANG1” and “ANG2”, which are angiopoietin-1 and angiopoietin-2, respectively, refer to the genes (ANG1: SEQ ID NO: 45, ANG2: SEQ ID NO: 47) represented by the polynucleotide sequences under GenBank Accession No. NM_{—}001146.3, and NM_{—}00111888.1, which are determined from their mRNA, respectively, and refer to the proteins (ANG1: SEQ ID NO: 46, GenBank Accession No. NP_{—}001137.2 and ANG2: SEQ ID NO: 48, GenBank Accession No. NP_{—}001112360.1), which are translated from the genes, respectively.

In the present invention, “SHC1” (src homology2 domain containing transforming protein 1) refers to the gene (SEQ ID NO: 5) represented by the polynucleotide sequence under GenBank Accession No. NM_{—}003029.4, which is determined from its mRNA, and refers to the protein (SEQ ID NO: 6) under GenBank Accession No. NP_{—}0030202, which is translated from the gene. The protein has an apoptosis-associated function.

In the present invention, “IL6” (interleukin 6), which is a cytokine playing an important role in hemogenesis and inflammation reactions, refers to the gene (SEQ ID NO: 7) represented by the polynucleotide sequence under GenBank Accession No. NM_{—}000600.3, which is determined from its mRNA, and refers to the protein (SEQ ID NO: 8) under GenBank Accession No. NP_{—}000591.1, which is translated from the gene. The protein has a function of controlling the JAK/STAT signaling pathway and the MAP kinase/ERKs signaling pathway.

In the present invention, “CXCR4” (CXC chemokine receptor 4, (also referred to as fusin)), which is an α-chemokine receptor specific to stroma-derived factor 1, refers to the gene (SEQ ID NO: 9) represented by the polynucleotide sequence under GenBank Accession No. NM_{—}001008540.1, which is determined from its mRNA, and refers to the protein (SEQ ID NO: 10) under GenBank Accession No. NP_{—}001008540.1, which is translated from the gene. The protein has a function of enhancing cell migration.

In the present invention, “COL4A3” (collagen, type IV, alpha 3), which is a component constituting extracellular matrix, refers to the gene (SEQ ID NO: 11) represented by the polynucleotide sequence under GenBank Accession No. NM_{—}000091.4, which is determined from its mRNA, and refers to the protein (SEQ ID NO: 12) under GenBank Accession No. NP_{—}000082.2, which is translated from the gene. The protein has a function of forming cytoskelton.

In the present invention, “NRP2” (neuropilin-2), which is a transmembrane receptor protein, refers to the gene (SEQ ID NO: 13) represented by the polynucleotide sequence under GenBank Accession No. NM_{—}003872.2, which is determined from its mRNA, and refers to the protein (SEQ ID NO: 14) under GenBank Accession No. NP_{—}003863.2, which is translated from the gene. The protein has a function of enhancing angiogenesis in a development stage and a tumorigenesis stage.

In the present invention, “MEIS1” (Meis homeobox 1), which is one of HOX genes, refers to the gene (SEQ ID NO: 15) represented by the polynucleotide sequence under GenBank Accession No. NM_{—}002398.2, which is determined from its mRNA, and refers to the protein (SEQ ID NO: 16) under GenBank Accession No. NP_{—}002389.1, which is translated from the gene. The protein has a function of controlling induced differentiation.

In the present invention, “ARHGAP22” (Rho GTPase activating protein 22), which is a molecule involved in intracellular signal transmission, refers to the gene (SEQ ID NO: 17) represented by the polynucleotide sequence under GenBank Accession No. NM_{—}021226.2, which is determined from its mRNA, and refers to the protein (SEQ ID NO: 18) under GenBank Accession No. NP_{—}067049.2, which is translated from the gene. The protein has a function of controlling remodeling of cytoskelton.

In the present invention, “SCG2” (secretogranin 2) refers to the gene (SEQ ID NO: 49) represented by the polynucleotide sequence under GenBank Accession No. NM_{—}003469.4, which is determined from its mRNA, and refers to the protein (SEQ ID NO: 50) under GenBank Accession No. NP_{—}003460.2, which is translated from the gene. The protein is a secretory protein having a function of enhancing cell migration.

In the present invention, “FGF9” (fibroblast growth factor 9), which is a secretory protein playing an important role in cell differentiation and functional maintenance, refers to the gene (SEQ ID NO: 51) represented by the polynucleotide sequence under GenBank Accession No. NM_{—}002010.2, which is determined from its mRNA, and refers to the protein (SEQ ID NO: 52) under GenBank Accession No. NP_{—}002001.1, which is translated from the gene. The protein has a function of interacting with FGFR3 (described later).

In the present invention, “PML” (promyelocytic leukemia), which is a type of transcription factor, refers to the gene (SEQ ID NO: 53) represented by the polynucleotide sequence under GenBank Accession No. NM_{—}002675.3, which is determined from its mRNA, and refers to the protein (SEQ ID NO: 54) under GenBank Accession No. NP_{—}002666.1, which is translated from the gene. The protein has a function of controlling cell-proliferation as a tumor suppressor.

In the present invention, “FGFR3” (fibroblast growth factor receptor 3), which is a protein having a function of enhancing cell-proliferation and differentiation, refers to the gene (SEQ ID NO: 55) represented by the polynucleotide sequence under GenBank Accession No. NM_{—}000142.3, which is determined from its mRNA, and refers to the protein (SEQ ID NO: 56) under GenBank Accession No. NP_{—}000133.1, which is translated from the gene. FGFR3 is known to have two isoforms, i.e., FGFR3b and FGFR3c.

In the present invention, “FGFR2” (fibroblast growth factor receptor 2), which is a protein having a function of enhancing cell-proliferation and differentiation, refers to the gene (SEQ ID NO: 57) represented by the polynucleotide sequence under GenBank Accession No. NM_{—}001144918.1, which is determined from its mRNA, and refers to the protein (SEQ ID NO: 58) under GenBank Accession No. NP_{—}001138390.1, which is translated from the gene.

In the present invention, “FGFR1” (fibroblast growth factor receptor 1), which is a protein having a function of enhancing cell-proliferation and differentiation, refers to the gene (SEQ ID NO: 59) represented by the polynucleotide sequence under GenBank Accession No. NM_{—}001174063.1, which is determined from its mRNA, and refers to the protein (SEQ ID NO: 60) under GenBank Accession No. NP_{—}001167534.1, which is translated from the gene.

In the present invention, “FGFR4” (fibroblast growth factor receptor 4) is a protein having a function of enhancing cell-proliferation and differentiation, refers to the gene (SEQ ID NO: 61) represented by the polynucleotide sequence under GenBank Accession No. NM_{—}002011.3, which is determined from its mRNA, and refers to the protein (SEQ ID NO: 62) under GenBank Accession No. NP_{—}002002.3, which is translated from the gene.

In the present invention, “VEGFR1” (vascular endothelial growth factor receptor 1), which is a protein having a function of enhancing cell-proliferation and differentiation and angiogenesis, refers to the gene (SEQ ID NO: 63) represented by the polynucleotide sequence under GenBank Accession No. NM_{—}001159920.1, which is determined from its mRNA, and refers to the protein (SEQ ID NO: 64) under GenBank Accession No. NP_{—}001153392.1, which is translated from the gene.

In the present invention, the “inhibitor” refers to a substance having an inhibitory activity against the function of a target molecule such as a compound, an antibody, an anti-sense oligonucleotide (“Antisense Drug Technology: Principles, Strategies, and Applications (Second Edition)”, CRC Press, 2007), an RNAi oligonucleotide (“RNA Methodologies (Third Edition)”, Elsevier, 2005, Chapter 24), a peptide nucleic acid (Kaihatsu et al., Chemistry & Biology 2004, 11(6), p. 749-758) and a peptidic antagonist (Ladner et al., Drug Discovery Today, 2004, 9, p. 525-529).

In the present invention, the “angiogenesis inhibitor” refers to a substance having an inhibitory activity against angiogenesis. The type of substance is not particularly limited as long as it has such an activity. Examples thereof include, but not limited to, a VEGF receptor inhibitor, an FGF receptor inhibitor, a PDGF receptor inhibitor, a RET kinase inhibitor, a KIT kinase inhibitor, an EGF inhibitor, an integrin inhibitor, a matrix metalloprotease inhibitor and an endogenous inhibitory substance; preferably include a VEGF receptor inhibitor, an FGF receptor inhibitor, a PDGF receptor inhibitor, an RET kinase inhibitor and a KIT kinase inhibitor; more preferably include a VEGF receptor-kinase inhibitor and an FGF receptor inhibitor; and most preferably, a VEGF receptor-kinase inhibitor.

If the angiogenesis inhibitor to be used in the present invention is a compound, it may form pharmacologically acceptable salts with acids or bases. The angiogenesis inhibitor of the present invention includes these pharmacologically acceptable salts. Examples of the salts with acids include, but not limited to, inorganic acid salts such as a hydrochloride, a hydrobromide, a sulfate and a phosphate; and organic acid salts such as formic acid, acetic acid, lactic acid, succinic acid, fumaric acid, maleic acid, malic acid, citric acid, tartaric acid, tosic acid, stearic acid, benzoic acid, mesyl acid, benzene sulfonic acid, p-toluene sulfonic acid and trifluoroacetic acid. Furthermore, examples of the salts with bases include, but not limited to, alkali metal salt such as a sodium salt and a potassium salt; alkaline earth metal salts such as a calcium salt and a magnesium salt, organic base salts such as trimethylamine, triethylamine, pyridine, picoline, dicyclohexyl amine, N,N-dibenzylethylenediamine, arginine, and lysine; and ammonium salts.

Furthermore, if the angiogenesis inhibitor to be used in the present invention is a compound, which has solvates and optical isomers, these solvates and optical isomers are included. As the solvates, e.g., hydrates and nonhydrates and preferably hydrates can be mentioned, but are not limited to these. Examples of solvents include, but not limited to, water, alcohol (for example, methanol, ethanol, n-propanol) and dimethylformamide.

Furthermore, in the present invention, if the angiogenesis inhibitor is a compound, the compound may be a crystal or amorphous. Furthermore, if there are crystal polymorphisms, a crystal form of any one of them and a mixture thereof may be used.

Furthermore, the angiogenesis inhibitor of the present invention includes an angiogenesis inhibitor, which is metabolized in a living body by oxidation, reduction, hydrolysis and/or conjugation. Furthermore, the angiogenesis inhibitor of the present invention also includes a compound, which is metabolized in a living body by oxidation, reduction, or hydrolysis to produce an angiogenesis inhibitor.

If the angiogenesis inhibitor to be used in the present invention is an antibody, examples of the antibody include a polyclonal antibody, a monoclonal antibody (Kohler et al., Nature, 1975, 256, p. 495-497), a chimeric antibody (Morrison et al., Proceedings of the National Academy of Sciences USA, 1984, 81, p. 6851-6855), single chain antibody (scFV) (Huston et al., Proceedings of the National Academy of Sciences USA, 1988, 85, p. 5879-5883.; Rosenburg et al. (Ed.), “The Pharmacology of Monoclonal Antibody, vol. 113”, Springer Verlag, 1994, p. 269-315), a humanized antibody (Jones et al., Nature, 1986, 321, p. 522-525), a polyspecific antibody (Millstein et al., Nature, 1983, 305, p. 537-539; Paulus, Behring Institute Mitteilungen, 1985, 78, p. 118-132; van Dijk et al., International Journal of Cancer 1989, 43, p. 344-349), a fully human antibody (McCafferty et al., Nature, 1990, 348, p. 552-554.; Lonberg et al., Nature, 1994, 368, p. 856-859.; Green et al., Nature Genetics, 1994, 7, p. 13-21) and antibody fragments such as Fab, Fab′, F(ab′)2, Fc, and Fv. Preferably, a monoclonal antibody is mentioned. Furthermore, the antibody of the present invention may be modified with e.g., polyethylene glycol (PEG), if necessary. Other than this, the antibody of the present invention can be produced as a fusion protein with e.g., β-galactosidase, MBP, GST or GFP such that the antibody can be detected without using a secondary antibody in e.g., ELISA. Furthermore, the antibody of the present invention may be modified such that it can be recovered by using e.g., avidin or streptoavidin by labeling the antibody with e.g., biotin.

The antibody of the present invention can be produced by using a target protein or a partial fragment thereof or using cells expressing it as a sensitizing antigen in accordance with a conventional method (“Current Protocols in Molecular Biology”, John Wiley & Sons, 2010, Chapter 11). In this case, the target protein or a partial fragment thereof may be a fusion protein with e.g., Fc region, GST, MBP, GFP and AP.

The target protein of the antibody of the present invention may be a biological molecule involved in angiogenesis or a receptor thereof. For example, a VEGF receptor inhibitor may be an anti-VEGF antibody and an anti-VEGF receptor antibody.

The polyclonal antibody and monoclonal antibody can be prepared by a method known to those skilled in the art (E. Harlow et al. (Ed.), “Antibodies: A Laboratory Manual”, Cold Spring Harbor Laboratory, 1988).

The polyclonal antibody can be obtained, for example, by administering an antigen to a mammal such as a mouse, a rabbit and a rat, taking blood from the mammal, separating and purifying an antibody from the taken blood. A method for immune sensitization, which is known to those skilled in the art, is carried out, for example, by administering an antigen once or more. Furthermore, an antigen (or a partial fragment thereof) can be used by dissolving it in an appropriate buffer solution, for example, an appropriate buffer solution containing an adjuvant generally used such as complete Freund adjuvant or aluminum hydroxide; however, an adjuvant may not be used depending upon the administration route, conditions and others.

In one to two months after the final immune sensitization, blood is taken from the mammal. The blood is subjected to a separation and purification process by a conventional method such as centrifugation, precipitation by ammonium sulfate or polyethylene glycol and/or various types of chromatographic methods. In this manner, a polyclonal antibody can be obtained as polyclonal antisera.

As a method for producing a monoclonal antibody, a hybridoma method may be mentioned. In the hybridoma method, first, a mammal is immunized in the same manner as in the production of a polyclonal antibody. Appropriate days after immunization, blood is partially collected and the titer of the antibody is preferably determined by a conventional method such as ELISA.

Next, the spleen is taken out from the immunized animal to obtain B cells. Subsequently, the B cells are fused with myeloma cells in accordance with a conventional method to prepare an antibody-producing hybridoma. The myeloma cells to be used are not particularly limited and myeloma cells conventionally known can be used. As the cell fusion method to be used, a conventional method in the art such as a Sendai virus method, a polyethylene glycol method and a protoplast method can be arbitrarily selected. The obtained hybridoma cells are cultured in HAT medium (medium containing hypoxanthine, aminopterin and thymidine) for an appropriate period in accordance with a conventional method to select hybridoma cells. Subsequently, desired antibody-producing hybridoma cells are screened and cloned.

As the screening method, a known antibody detection method such as ELISA and radioimmunoassay can be used. Furthermore, as the cloning method, a conventional method in the art can be used. For example, the limiting dilution method and FACS can be used. The obtained hybridoma cells are cultured in an appropriate culture solution or injected, for example, in a mouse abdominal cavity having compatibility with the hybridoma cells. From the culture solution or ascitic fluid, a desired monoclonal antibody can be isolated and purified by e.g., salting out, ion exchange chromatography, gel filtration and/or affinity-chromatography. Furthermore, the isotype of the antibody of the present invention is not particularly limited.

The antibody of the present invention is preferably a neutralizing antibody capable of inhibiting vascular endothelial growth activity of a target protein by recognizing and binding to the target protein or a partial fragment thereof.

Specific examples of the angiogenesis inhibitor preferably used in the present invention are as follows. They can be produced or obtained in accordance with the method described in respective literatures.

### 4-(3-Chloro-4-(cyclopropylaminocarbonyl)aminophenoxy)-7-methoxy-6-quinolinecarboxamide (Formula (IV))

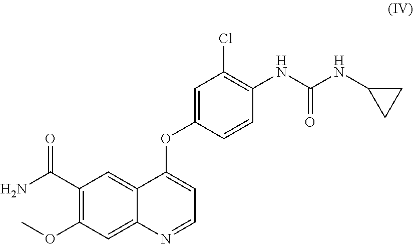 [SMILES: COc1cc2nccc(Oc3ccc(NC(=O)NC4CC4)c(Cl)c3)c2cc1C(N)=O]

The compound can be produced by the method described in WO02/032872. The form of the compound is preferably a methanesulfonate but not limited to this. The compound in the form of a mesylate is referred also to as “E7080”. The compound is known to have an inhibitory activity against a receptor tyrosine kinase such as VEGF receptor, FGF receptor, RET kinase and KIT kinase (WO2007/136103, Matsui et al., Clinical Cancer Research, 2008, 14 (17), p. 5459-5465).

### N-(4-Bromo-2-fluorophenyl)-6-methoxy-7-[2-(1H-1,2,3-triazol-1-yl)ethoxy]quinazoline-4-amine

The compound, which is referred also to as “ZD4190”, can be produced by the method described in Hennequin et al., Journal of Medicinal Chemistry, 1999, 42, p. 5369-5389. The compound is known to have a VEGF receptor inhibitory activity (Wedge et at, Cancer Research, 2000, 60, p. 970-975).

### N-(4-Bromo-2-fluorophenyl)-6-methoxy-7-[(1-methylpiperidin-4-yl)methoxy]quinazoline-4-amine (Formula (3))

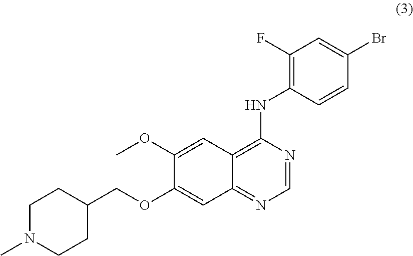 [SMILES: COc1cc2c(Nc3ccc(Br)cc3F)ncnc2cc1OCC1CCN(C)CC1]

The compound, which is referred also to as “ZD6474” or “vandetanib”, can be produced by the method described in Hennequin et al, Journal of Medicinal Chemistry, 2002, 45, p. 1300-1312. Furthermore, the compound is known to have a VEGF receptor inhibitory activity

### 3-[(2,4-Dimethylpyrrol-5-yl) methylene]-2-indolinone (Formula (4))

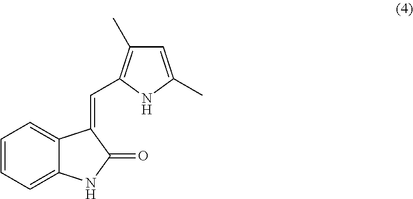 [SMILES: Cc1cc(C)c(/C=C2\C(=O)Nc3ccccc32)[nH]1]

The compound, which is referred also to as “SU5416” or “semaxanib” can be produced by the method described in Sun et at, Journal of Medicinal. Chemistry, 1998, 41, p. 2588-2603., U.S. Pat. No. 5,792,783. The compound is known to have a VEGF receptor inhibitory activity (Fong et at, Cancer Research., 1999, 59, p. 99-106).

### (Z)-3-[(2,4-dimethyl-5-(2-oxo-1,2-dihydroindol-3-ylidenemethyl)-1H-pyrrol-3-yl)-propionic acid (Formula (5))

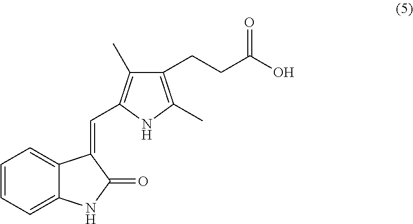 [SMILES: Cc1[nH]c(/C=C2\C(=O)Nc3ccccc32)c(C)c1CCC(=O)O]

The compound, which is referred also to as “SU6668”, can be produced by the method described in Sun et al., Journal of Medicinal Chemistry, 1999, 42, p. 5120-5130. The compound is known to have inhibitory activities against VEGF receptor, FGF receptor and PDGF receptor (Laird et al., Cancer Research, 2000, 60, p. 4152-4160).

### 5-(5-Fluoro-2-oxo-1,2-dihydroindol-3-ylidenemethyl)-2,4-dimethyl-1H-pyrrole-3-carboxylic acid (2-diethylaminoethyl)amide (Formula (6))

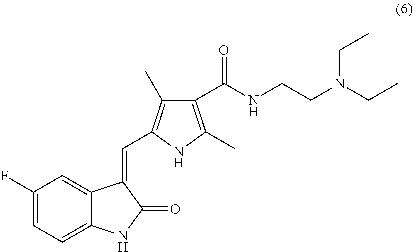 [SMILES: CCN(CC)CCNC(=O)c1c(C)[nH]c(/C=C2\C(=O)Nc3ccc(F)cc32)c1C]

The compound, which is referred also to as “SU11248”, or “sunitinib” can be produced by the method described in Sun et al., Journal of Medicinal Chemistry, 2003, 46, p. 1116-1119. The form to be taken by the compound is preferably a malate; but not limited to this. The compound is known to have inhibitory activities against VEGF receptor, PDGF receptor, KIT kinase and FLT3 kinase (Mendel et al., Clinical Cancer Research, 2003, 9, p. 327-337). Furthermore, the compound has been approved as a therapeutic agent for gastrointestinal stromal tumor (GIST) and renal cell cancer under the name of Sutent (registered trade mark) and can be obtained from the commercially available product

### N,N-Dimethylglycine 3-{5,6,7,13-tetrahydro-9-[(1-methylethoxy)methyl]-5-oxo-12H-indeno(2,1-a)pyrrolo(3,4-c)carbazol-12-yl}propyl ester (Formula (7))

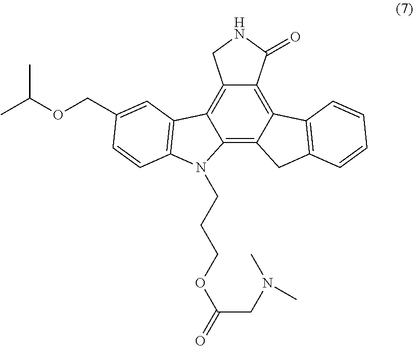 [SMILES: CC(C)OCc1ccc2c(c1)c1c3c(c4c(c1n2CCCOC(=O)CN(C)C)Cc1ccccc1-4)C(=O)NC3]

The compound, which is referred also to as “CEP-7055”, can be produced by the method described in Gingrich et al., Journal of Medicinal Chemistry 2003, 46, p. 5375-5388. The compound is known to have a VEGF receptor inhibitory activity

### 3-(4-Bromo-2,6-difluoro-benzyloxy)-5-[3-(4-pyrrolidin-1-yl-butyl)-ureido]-isothiazole-4-carboamide (Formula (8))

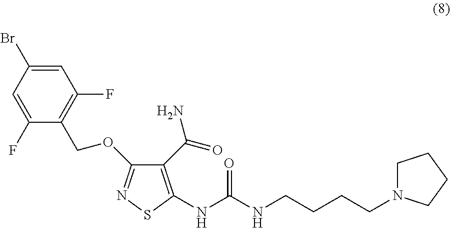 [SMILES: NC(=O)c1c(OCc2c(F)cc(Br)cc2F)nsc1NC(=O)NCCCCN1CCCC1]

The compound, which is referred also to as “CP-547,632”, can be produced by the method described in WO99/62890. The compound is known to have a VEGF receptor inhibitory activity (Beebe et. al., Cancer Research, 2003, 63, p. 3301-7309).

### N-{2-Chloro-4-[(6,7-dimethoxy-4-quinazolinyl)oxy]phenyl}-N′-propylurea (Formula (9))

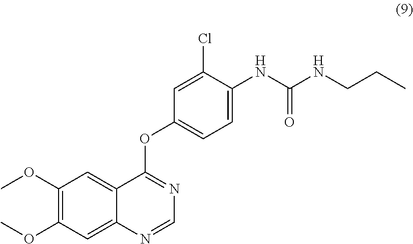 [SMILES: CCCNC(=O)Nc1ccc(Oc2ncnc3cc(OC)c(OC)cc23)cc1Cl]

The compound, which is referred also to as “KRN633”, can be produced by the method described in WO00/43366. The compound is known to have a VEGF receptor inhibitory activity (Nakamura et. al., Molecular Cancer Therapeutics, 2004, 3, p. 1639-1649).

### 1-(4-Chloroanilino)-4-(4-pyridylmethyl)phthalazine (Formula (10))

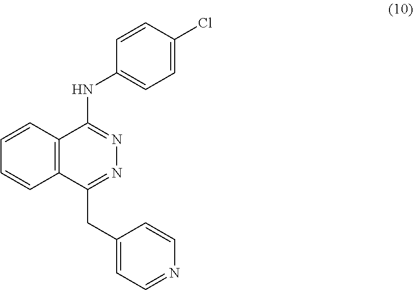 [SMILES: Clc1ccc(Nc2nnc(Cc3ccncc3)c3ccccc23)cc1]

The compound, which is referred also to as “PTK787/ZK 222584” or “vatalanib”, can be produced by the method described in WO98/35958. The compound is known to have a VEGF receptor inhibitory activity (Wood et al., Cancer Research, 2000, 60, p. 2178-2189).

### N-{2-chloro-4-[(6,7-dimethoxy-4-quinolyl)oxy]phenyl}-N′-(5-methyl-3-isoxazolyl)urea (Formula (11))

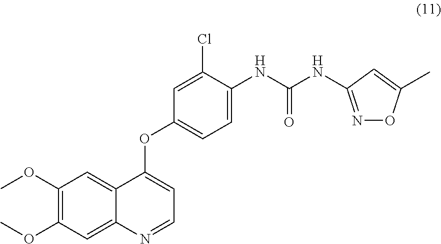

The compound, which is referred also to as “KRN951”, can be produced by the method described in WO02/088110. The compound is known to have inhibitory activities against VEGF receptor, PDGF receptor, and KIT kinase (Nakamura et al., Cancer Research, 2006, 66, p. 9134-9142).

### 4-[(4-Fluoro-2-methylindol-5-yl)oxy]-6-methoxy-7-[3-(pyrrolidin-1-yl) propoxy]quinazoline (Formula (12))

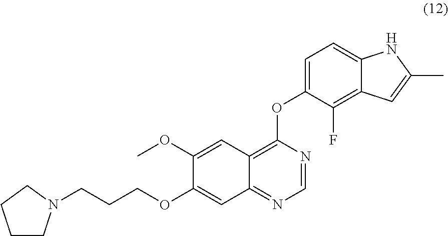 [SMILES: COc1cc2c(Oc3ccc4[nH]c(C)cc4c3F)ncnc2cc1OCCCN1CCCC1]

The compound, which is referred also to as “AZD2171” or “cediranib”, can be produced by the method described in WO00/47212. The compound is known to have a VEGF receptor inhibitory activity (Cancer Research, 2005, 65, p. 4389-4400).

### N-Methyl-2-[[3-[(E)-2-(2-pyridyl)ethenyl]-1H-indazol-6-yl]thio]benzamide (Formula (13))

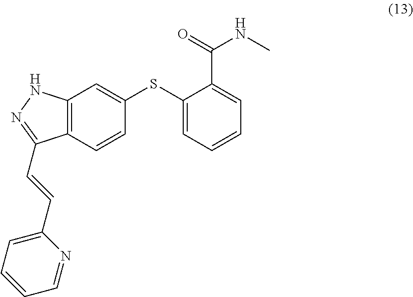 [SMILES: CNC(=O)c1ccccc1Sc1ccc2c(/C=C/c3ccccn3)n[nH]c2c1]

The compound, which is referred also to as “AG-013736” or “axitinib”, can be produced by the method described in WO01/02369. The compound is known to have a VEGF receptor inhibitory activity (Kelly et al., Targeted Oncology, 2009, 4, p. 297-305).

### 5-((Z)-(5-Fluoro-2-oxo-1,2-dihydro-3H-indol-3-ylidene)methyl)-N-((2S)-2-hydroxy-3-morpholin-4-ylpropyl)-2,4-dimethyl-1H-pyrrole-3-carboxamide (Formula (14))

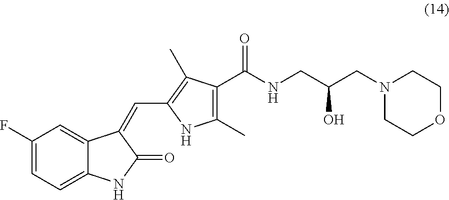 [SMILES: Cc1[nH]c(/C=C2\C(=O)Nc3ccc(F)cc32)c(C)c1C(=O)NC[C@H](O)CN1CCOCC1]

The compound, which is referred also to as “SU14813”, can be produced by the method described in U.S. Pat. No. 6,653,308. The compound is known to have inhibitory activities against VEGF receptor, PDGF receptor, KIT kinase and FLT kinase (Patyna et al., Molecular Cancer Therapy 2006, 5, p. 1774-1782).

### 3-((Quinolin-4-ylmethyl)amino)-N-(4-(trifluoromethoxy)phenyl)thiophene-2-carboxamide (Formula (15))

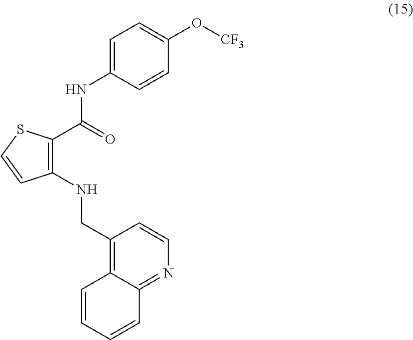 [SMILES: O=C(Nc1ccc(OC(F)(F)F)cc1)c1sccc1NCc1ccnc2ccccc12]

The compound, which is referred also to as “OSI-930”, can be produced by the method described in WO2004/063330. The compound is known to have inhibitory activities against VEGF receptor, PDGF receptor, and KIT kinase (Petti et al., Molecular Cancer Therapeutics, 2005, 4, p. 1186-1197).

### 6-(2,6-dichlorophenyl)-8-methyl-2-phenylamino-8H-pyrid[2,3-d]pyrimidine-7-one (Formula (16))

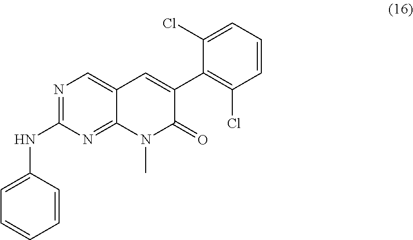 [SMILES: Cn1c(=O)c(-c2c(Cl)cccc2Cl)cc2cnc(Nc3ccccc3)nc21]

The compound, which is referred also to as “TKI-28”, is known to have inhibitory activities against VEGF receptor, EGF receptor, PDGF receptor, KIT kinase, ErbB-2 and Src kinase (Guo et al., Cancer Biology & Therapy., 2005, 4, p. 1119-1126).

### 2-((1,6-Dihydro-6-oxo-pyridin-3-ylmethyl)amino)-N-(3-(trifluoromethyl)phenyl)-3-pyridine-carboxamide (Formula (17))

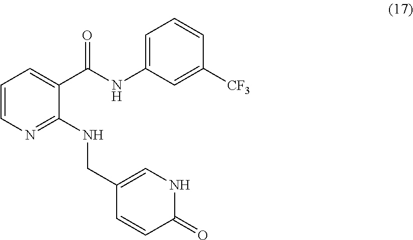 [SMILES: O=C(Nc1cccc(C(F)(F)F)c1)c1cccnc1NCc1ccc(=O)[nH]c1]

The compound, which is referred also to as “ABP309”, can be produced by the method described in WO01/55114. The compound is known to have inhibitory activities against e.g., VEGF receptor, PDGF receptor and KIT kinase (Brueggen et al., EJC Supplements, 2004, 2, p. 8 (Abs 172)).

### 4-(4-(4-Chloro-phenylamino)-furo[2,3-d]pyridazin-7-yloxymethyl)-pyridine-2-carboamide (Formula (18))

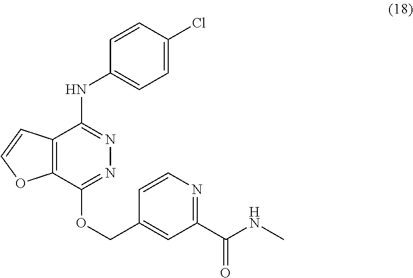 [SMILES: CNC(=O)c1cc(COc2nnc(Nc3ccc(Cl)cc3)c3ccoc23)ccn1]

The compound, which is referred also to as “BAY 57-9352” or “telatinib”, can be produced by the method described in WO01/23375. The compound is known to have inhibitory activities against VEGF receptor, PDGF receptor, and KIT kinase (Eskers et al., Journal of Clinical Oncology, 2009, 27, p. 4169-4176).

### N-(4-chloro-3-(trifluoromethyl)phenyl)-N′-(4-(2-(N-methylcarbamoyl)-4-pyridyloxy)phenylurea (Formula (19))

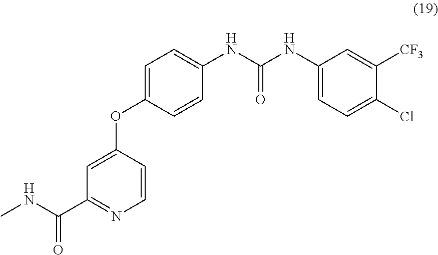 [SMILES: CNC(=O)c1cc(Oc2ccc(NC(=O)Nc3ccc(Cl)c(C(F)(F)F)c3)cc2)ccn1]

The compound, which is referred also to as “BAY 43-9006” or “sorafenib”, can be produced by the method described in U.S. Pat. No. 7,235,576. The form to be taken by the compound is preferably a tosylate, but not limited to this. The compound is known to have inhibitory activities against VEGF receptor, PDGF receptor, KIT kinase and FLT3 kinase (Wilhelm et al., Cancer Research, 2004, 64, p. 7099-7109). Furthermore, the compound has been approved under the name of Nexavar (registered trade mark) as a therapeutic agent for liver cell cancer and renal cell cancer, and can be obtained from the commercially available product.

### 4-Amino-5-fluoro-3-(6-(4-methyl-piperazin-1-yl)-1H-benzimidazol-2-yl)-1H-quinolin-2-one (Formula (20))

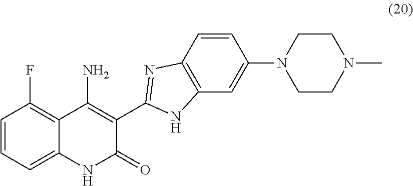 [SMILES: CN1CCN(c2ccc3nc(-c4c(N)c5c(F)cccc5[nH]c4=O)[nH]c3c2)CC1]

The compound, which is referred also to as “CHIR-258” or “dovitinib”, can be produced by the method described in WO02/22598. The compound is known to have inhibitory activities against VEGF receptor, FGF receptor, PDGF receptor, CSF-1 receptor, KIT kinase, and FLT3 kinase (Trudel et al., Blood, 2005, 105, p. 2941-2948).

### 4-(4-(1-Amino-1-methyl-ethyl)-phenyl)-2-(4-(2-morpholin-4-yl-ethyl)-phenylamino)-pyrimidine-5-carbonitrile (Formula (21))

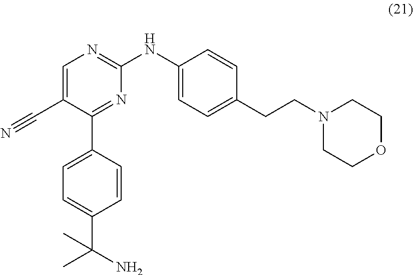 [SMILES: CC(C)(N)c1ccc(-c2nc(Nc3ccc(CCN4CCOCC4)cc3)ncc2C#N)cc1]

The compound, which is referred also to as “JNJ17029259”, can be produced by the method described in Reuman et al., Journal of Organic Chemistry, 2008, 73, p. 1121-1123. The compound is known to have inhibitory activities against VEGF receptor, FGF receptor, PDGF receptor, and FLT3 kinase (Emanuel et al., Molecular Pharmacology, 2004, 66, p. 635-647).

### [6-[4-[(4-Ethylpiperain-1-yl)methyl]phenyl]-7H-pyrrolo[2,3-d]pyrimidin-4-yl]-((R)-1-phenylethyl)amine (Formula (22))

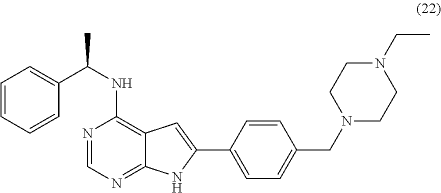 [SMILES: CCN1CCN(Cc2ccc(-c3cc4c(N[C@H](C)c5ccccc5)ncnc4[nH]3)cc2)CC1]

The compound, which is referred also to as “AEE-788”, can be produced by the method described in WO03/013541. The compound is known to have inhibitory activities against VEGF receptor, FGF receptor, and EGF receptor (Traxler et al., Cancer Research, 2004, 64, p. 4931-4941).

### 9-(1-Methylethoxy)methyl-12-(3-hydroxypropyl)-6H,7H,13H-indeno[2,1-a]pyrrolo[3,4-c]carbazol-5-one (Formula (23))

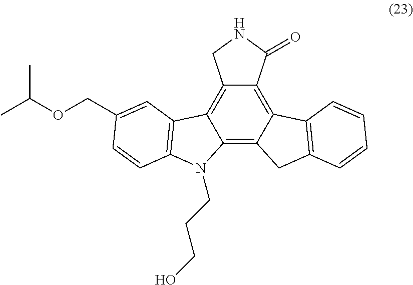 [SMILES: CC(C)OCc1ccc2c(c1)c1c3c(c4c(c1n2CCCO)Cc1ccccc1-4)C(=O)NC3]

The compound, which is referred also to as “CEP-5214”, can be produced by the method described in WO02/17914. The compound is known to have a VEGF receptor inhibitory activity (Ruggeri et al., Cancer Research, 2003, 63, p. 5978-5991).

### N-(2,4-Difluorophenyl)-N′-{4-[(6,7-dimethoxy-4-quinolyl)-oxy]-2-fluorophenyl}urea (Formula (24))

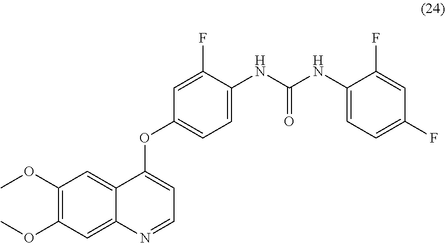 [SMILES: COc1cc2nccc(Oc3ccc(NC(=O)Nc4ccc(F)cc4F)c(F)c3)c2cc1OC]

The compound, which is referred also to as “Ki8751”, can be produced by the method described in WO00/43366. The compound is known to have inhibitory activities against VEGF receptor, PDGF receptor, and KIT kinase (Kubo et al., Journal of Medicinal Chemistry, 2005, 48, p. 1359-1366).

### N-[4-(3-Amino-1H-indazol-4-yl)phenyl]-N′-(2-fluoro-5-methylphenyl)urea (Formula (25))

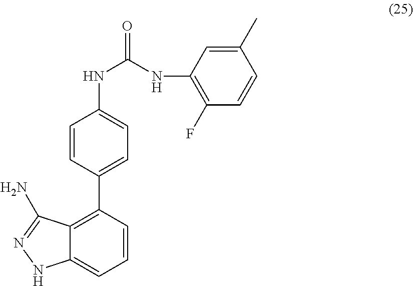 [SMILES: Cc1ccc(F)c(NC(=O)Nc2ccc(-c3cccc4[nH]nc(N)c34)cc2)c1]

The compound, which is referred also to as “ABT-869” or “linifanib”, can be produced by the method described in WO2004/113304. The compound is known to have inhibitory activities against VEGF receptor, PDGF receptor, CSF-1 receptor, KIT kinase, and FLT3 kinase (Guo et al., Molecular Cancer Therapy, 2006, 5, p. 1007-1013).

### 2-Methyl-6-[2-(1-methyl-1H-imidazol-2-yl)-thieno[3,2-b]pyridin-7-yloxy]-benzo[b]thiophene-3-carboxylic acid methyl amide (Formula (26))

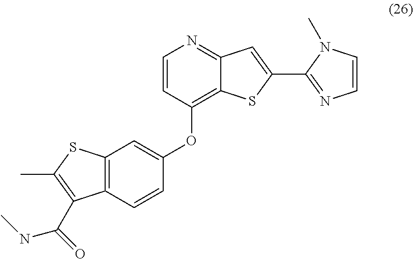 [SMILES: CNC(=O)c1c(C)sc2cc(Oc3ccnc4cc(-c5nccn5C)sc34)ccc12]

The compound, which is referred also to as “AG-028262”, can be produced by the method described in WO03/106462. The compound is known to have a VEGF receptor inhibitory activity (Mancuso et al., Journal of Clinical Investigation, 2006, 116, p. 2610-2621).

### (R)-1-(4-(4-Fluoro-2-methyl-1H-indol-5-yloxy)-5-methylpyrrolo[1,2-f][1,2,4]triazin-6-yloxy)propan-2-ol (Formula (27))

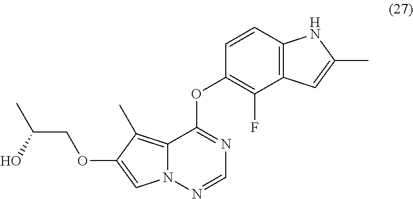 [SMILES: Cc1cc2c(F)c(Oc3ncnn4cc(OC[C@@H](C)O)c(C)c34)ccc2[nH]1]

The compound, which is referred also to as “BMS-540215” or “brivanib”, can be produced by the method described in WO2004/009601. The compound is known to have a VEGF receptor inhibitory activity (Bhide et al., Journal of Medicinal Chemistry, 2006, 49, p. 2143-2146).

### (S)—((R)-1-(4-(4-Fluoro-2-methyl-1H-indol-5-yloxy)-5-methylpyrrolo[1,2-f][1,2,4]triazin-6-yloxy)propan-2-ol)2-aminopropanoate (Formula (28))

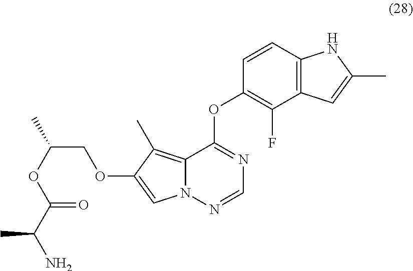 [SMILES: Cc1cc2c(F)c(Oc3ncnn4cc(OC[C@@H](C)OC(=O)[C@H](C)N)c(C)c34)ccc2[nH]1]

The compound, which is referred also to as “BMS-582664” or “brivanib alaninate”, can be produced by the method described in WO2004/009601. The compound is known to have a VEGF receptor inhibitory activity (Bhide et al., Journal of Medicinal Chemistry, 2006, 49, p. 2143-2146).

### 3-[(4-Morpholin-4-yl-phenylamino)-methylene]-1,3-dihydroindol-2-one (Formula (29))

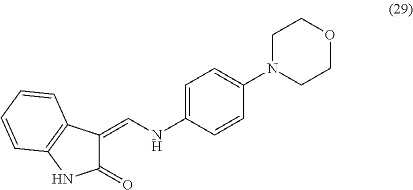 [SMILES: O=C1Nc2ccccc2/C1=C/Nc1ccc(N2CCOCC2)cc1]

The compound, which is referred also to as “AGN-199659”, can be produced by the method described in WO03/027102. The compound is known to have a VEGF receptor inhibitory activity

### 5-[[4-[(2,3-Dimethyl-2H-indazol-6-yl)methylamino]pyrimidin-2-yl]amino]-2-methylbenzenesulfonamide (Formula (30))

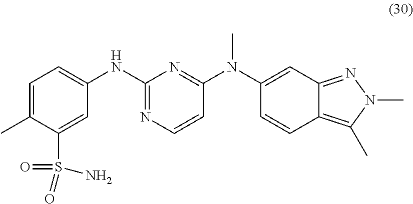

The compound, which is referred also to as “GW-786034” or “pazopanib”, can be produced by the method described in WO02/059110. The compound is known to have inhibitory activities against VEGF receptor, PDGF receptor, and KIT kinase (Bukowski et al., Nature Reviews Drug Discovery, 2010, 9, p. 17-18).

### (3Z)-3-[6-(2-Morpholin-4-ylethoxy)quinolin-2(1H)-ylidene]-1,3-dihydro-2H-indol-2-one (Formula (31))

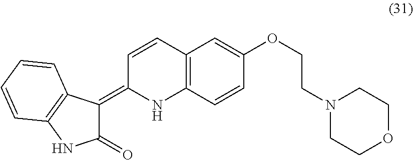 [SMILES: O=C1Nc2ccccc2/C1=C1\C=Cc2cc(OCCN3CCOCC3)ccc2N1]

The compound, which is referred also to as “YM-231146”, is known to have a VEGF receptor inhibitory activity (Amino et al., Biological and Pharmaceutical Bulletin, 2005, 28, p. 2096-2101).

### 2-((2-((4-(4-(4-(tert-Butyl)anilino)phenoxy)-6-methoxy-7-quinolyl)oxy)ethyl)amino)-1-ethanol (Formula (32))

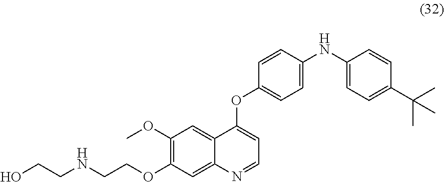 [SMILES: COc1cc2c(Oc3ccc(Nc4ccc(C(C)(C)C)cc4)cc3)ccnc2cc1OCCNCCO]

The compound, which is referred also to as “Ki23057”, can be produced by the method described in WO03/033472. The compound is known to have inhibitory activities against VEGF receptor, FGF receptor, PDGF receptor, and KIT kinase (Shimizu et at, Bioorganic & Medicinal Chemistry Letters, 2004, 14, p. 875-879).

“Bevacizumab”

Bevacizumab is an anti-VEGF humanized monoclonal antibody and binds to VEGF to inhibit the binding of VEGF to VEGF receptor. The antibody can be produced by the method described in WO94/10202. The antibody has been approved under the name of Avastin (registered trade mark) as a therapeutic agent for colorectal cancer, non-small-cell lung cancer, breast cancer, glioblastoma and renal cell cancer and can be obtained from the commercially available product.

### 1-[2-Amino-6-(3,5-dimethoxyphenyl)-pyrido(2,3-d)pyrimidin-7-yl]-3-tert-butyl urea (Formula (33)

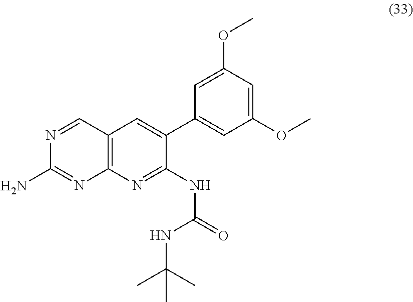 [SMILES: COc1cc(OC)cc(-c2cc3cnc(N)nc3nc2NC(=O)NC(C)(C)C)c1]

The compound, which is referred also to as “PD 166866”, can be produced by the method described in Hamby et al., Journal of Medicinal Chemistry, 1997, 40, p. 2296-2303. The compound is known to have an FGF receptor inhibitory activity

### 1-tert-Butyl-3-[2-(4-diethylamino)butylamino-6-(3,5-dimethoxyphenyl)-pyrido(2,3-d)pyrimidin-7-yl]urea (Formula (34))

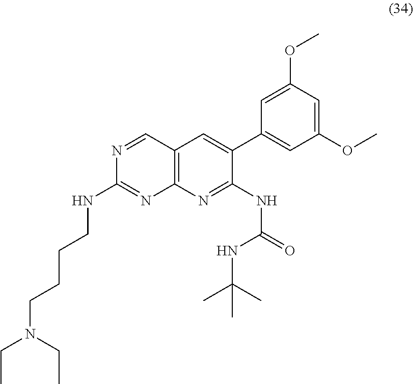 [SMILES: CCN(CC)CCCCNc1ncc2cc(-c3cc(OC)cc(OC)c3)c(NC(=O)NC(C)(C)C)nc2n1]

The compound, which is referred also to as “PD173074”, can be produced by the method described in U.S. Pat. No. 5,733,913. The compound is known to have an FGF receptor inhibitory activity (Mohammadi et al., EMBO J., 1998, 17, p. 5896-5904).

### 4-[4-[N-(4-Nitrophenyl)carbarnoyl]-1-piperazinyl]-6,7-dimethoxyquinazoline (Formula (35))

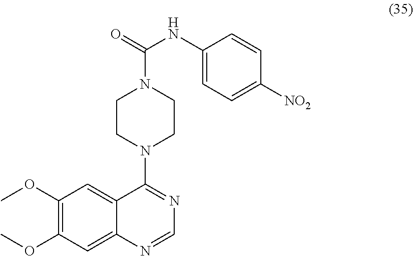 [SMILES: COc1cc2ncnc(N3CCN(C(=O)Nc4ccc([N+](=O)[O-])cc4)CC3)c2cc1OC]

The compound, which is referred also to as “CT52923”, can be produced by the method described in WO98/14437. The compound is known to have inhibitory activities against PDGF receptor, and KIT kinase (Yu et al., Journal of Pharmacology and Experimental Therapeutics, 2001, 298, p. 1172-1178).

### 3-Z-[1-(4-(N-((4-Methylpipemzine-1-yl)methylcarbonyl)-N-methylamino)anilino)-1-phenylmethylene]-6-methoxycarbonyl-2-indolinone (Formula (36))

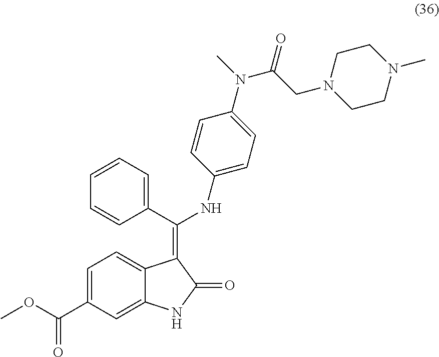

The compound, which is referred also to as “BIBF 1120” or “intedanib”, can be produced by the method described in WO01/27081. The compound is known to have inhibitory activities against e.g., VEGF receptor, FGF receptor, PDGF receptor, KIT kinase, FLT3 kinase and Lck (Hillberg et al., Cancer Research, 2008, 68, p. 4774-4782).

### N-(3,3-dimethylindolin-6-yl){2-[(4-pyridylmethyl)amino](3-pyridyl)}carboxamide (Formula (37))

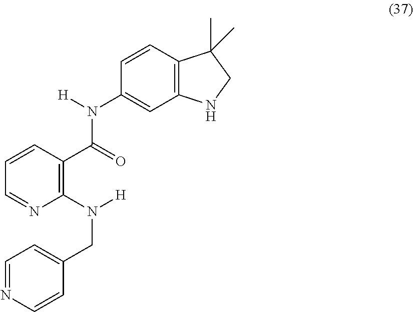 [SMILES: CC1(C)CNc2cc(NC(=O)c3cccnc3NCc3ccncc3)ccc21]

The compound, which is referred also to as “AMG706” or “motesanib”, can be produced by the method described in U.S. Pat. No. 6,878,714. The compound is known to have inhibitory activities against VEGF receptor, PDGF receptor, RET kinase and KIT kinase (Polverino et al., Cancer Research, 2006, 66, p. 8715-8721).

Other examples of the angiogenesis inhibitor in the present invention include “PI-88” (referred also to as “muparfostat”. WO96/33726; McKenzie et al., British Journal of Pharmacology, 2007, 151, p. 1-14), VEGF trap (referred also to as “AVE-0005” or “aflibercept”. WO00/75319; Tew et al., Clinical Cancer Research, 2010, 16, p. 358-366), “RPI-4610” (referred also to as “Angiozyme (registered trade mark)”. U.S. Pat. No. 5,180,818; U.S. Pat. No. 6,346,398), 2-(8-hydroxy-6-methoxy-1-oxo-1H-2-benzopyran-3-yl)propionic acid (referred also to as “NM-3”. WO97/48693; Agata et al., Cancer Chemotherapy & Pharmacology, 2005, 56, p. 610-614), “IMC-1121b” (referred also to as “ramucirumab”. U.S. Pat. No. 6,811,779; Journal of Clinical Oncology, 2010, 28, p. 780-787.) and “IMC-18F1” (WO95/21868; Wu et al., Clinical Cancer Research, 2006, 12, p. 6573-6584).

Examples of the angiogenesis inhibitor of the present invention include preferably 4-(3-chloro-4-(cyclopropylaminocarbonyl)aminophenoxy)-7-methoxy-6-quinolinecarboxamide, 5-(5-fluoro-2-oxo-1,2-dihydroindol-3-ylidenemethyl)-2,4-dimethyl-1H-pyrrole-3-carboxylic acid (2-diethylaminoethyl)amide, 4-[(4-fluoro-2-methylindol-5-yl)oxyl-6-methoxy-7-[3-(pyrrolidin-1-yl)propoxy]quinazoline, N-methyl-2-[[3-[(E)-2-(2-pyridyl)ethenyl]-1H-indazol-6-yl]thio]benzamide, N-(4-chloro-3-(trifluoromethyl)phenyl)-N′-(4-(2-(N-methylcarbamoyl)-4-pyridyloxy)phenylurea, 5-[[4-[(2,3-dimethyl-2H-indazol-6-yl)methylamino]pyrimidin-2-yl]amino]-2-methylbenzenesulfonamide and bevacizumab or a pharmacologically acceptable salt thereof; and particularly preferably, 4-(3-chloro-4-(cyclopropylaminocarbonyl)aminophenoxy)-7-methoxy-6-quinolinecarboxamide or a pharmacologically acceptable salt thereof. As a pharmacologically acceptable salt of 4-(3-chloro-4-(cyclopropylaminocarbonyl)aminophenoxy)-7-methoxy-6-quinolinecarboxamide, preferably mesylate of 4-(3-chloro-4-(cyclopropylaminocarbonyl)aminophenoxy)-7-methoxy-6-quinolinecarboxamide is mentioned.

A method for determining the presence or absence of a mutation or loss of expression in B-Raf and PTEN in a sample derived from a tumor tissue of a subject will be described below.

A tumor tissue can be obtained by excising out from a subject, for example, by a surgical procedure (for example, biopsy). The size of the tumor tissue taken from a subject is not limited as long as a mutation or loss of expression of B-Raf and PTEN in the tumor tissue can be determined. For example, in the case of solid cancer, the size of the tumor tissue taken by biopsy (for example, 2 to 3 mm) is acceptable, and the size of a tissue piece cut by scalpel is acceptable. The size is not limited.

Furthermore, the tumor tissue may be specific cells further excised from the tissue piece taken out, by e.g., a method such as a laser capture micro-dissection method (Murray et al. (Ed.), “Laser Capture Microdissection; Methods and Protocols”, Humana Press, 2004).

Furthermore, blood is taken from a subject. Cancer cells circulating through the peripheral blood are isolated by the method of e.g., Kitago et al. From the cancer cell, a mutation or loss of expression of B-Raf and PTEN can be detected (Kitago et al., Clinical Chemistry, 2009, 55 (4), p. 757-764).

Furthermore, by use of a highly sensitivity nucleic acid detection method such as a gap-ligase chain reaction (GLCR) method, a mutation or loss of expression of B-Raf and PTEN can be directly detected from DNA circulating through the blood (Chiang et al., Head and Neck, 2010, 32, p. 229-234).

A mutation or loss of expression of B-Raf and PTEN can be detected by a conventional method such as a method of determining a nucleic acid sequence, a method using a nucleic acid or a specific antibody as a probe and a method using mass spectrometry; however a method of bringing a sample derived from a tumor tissue of a subject into contact with a probe is preferable. As a probe for detecting a mutation or loss of expression of B-Raf and PTEN, a nucleic acid probe or specific antibody to B-Raf or PTEN is mentioned. The “bringing into contact with” means that a sample derived from a tumor tissue of a subject and a probe are allowed to be present under conditions at which the sample derived from a tumor tissue of a subject and the probe can react with each other, for example, by mixing a sample and a probe and hybridizing a sample with a probe, although the method is not limited to these.

In detecting a mutation and loss of expression by determining a nucleotide sequence, the mutation or loss of expression of B-Raf and PTEN can be detected by subjecting a high-molecular DNA product (referred also to as an extracted high-molecular DNA), which is extracted from a sample or a product obtained by amplifying it by a polymerase chain reaction (PCR), to a direct nucleotide sequence determination method, Southern blot method, Northern blot method, a PCR-strand conformation polymorphism (PCR-SSCP) method, an allelic gene specific oligonucleotide probe (ASO) method, a direct gel assay method, Amplification Refractory Mutation System (ARMS) method, a dot blot analysis method using a mutation specific oligomer or an analogous method thereof. Furthermore, the mutation or loss of expression of B-Raf and PTEN can be detected also by a next generation sequencer such as Applied Biosystems 3730 DNA Analyzer (Applied Biosystems), Roche 454 Genome Sequencer FLX System (Roche), Genome Analyzer II (Illumina), Applied Biosystems SOLiD System (Applied Biosystems) and HeliScope Single Molecule Sequencer (Helicos) (“Current Protocols in Molecular Biology”, John Wiley & Sons, 2010, Chapter 7). As the nucleic acid probe to be used for detecting the mutation or loss of expression of B-Raf, for example, primers (SEQ ID NOs: 39 to 44) used in Example 1 are mentioned but not limited to these. As the nucleic acid probe to be used for detecting a mutation or loss of expression of PTEN, for example, primers (SEQ ID NOs: 19 to 38) used in Example 1 are mentioned but not limited to these.

In detecting a mutation and loss of expression by use of the Sanger method, genomic DNA is extracted from a sample derived from a subject in accordance with a conventional method and the obtained genomic DNA is subjected to PCR to amplify exon regions of B-Raf and PTEN. The amplified DNA is subjected to 1% agarose gel electrophoresis. After it is confirmed that the amplified DNA has a desired length, the PCR product is recovered from the gel and purified. The purified product is sequenced by a sequencer. In this manner, information such as gene mutation can be obtained.

The case where detection of a mutation and loss of expression is performed by a next generation sequencer will be described below.

For example, when Genome Analyzer II manufactured by Illumina is used as a next generation sequencer, RNA is extracted from a sample derived from a subject in accordance with a conventional method and cDNA is prepared based on the extracted RNA. The extracted RNA can be quantified by a technique such as Northern blot analysis, DNA microarray, RT-PCR and quantitative PCR As a preferable quantitative method for RNA, DNA microarray and quantitative PCR are mentioned; however, the quantitative method is not limited to the above methods.

The cDNA prepared is cut into fragments of about 200 bp suitable for analysis by a next generation sequencer and an adaptor sequence is added to prepare a cDNA library. The library prepared is allowed to bind onto a flow cell via the adaptor sequence to form a cluster. To the cluster, a sequence primer is added and a step of detecting fluorescence is repeatedly performed. In this manner, acquisition of data and analysis for a single base extension are performed to obtain information such as gene mutation.

When a mutation and loss of expression are detected by using a specific antibody as a probe, if a partial peptide having the amino acid sequence of a wild type and a partial peptide having the amino acid sequence of a mutant with respect to each of the mutation sites of B-Raf and PETN, are respectively used as antigens, antibodies specific to the mutation sites can be prepared by a conventional method. When a loss of expression is detected, if a partial peptide having a wild-type amino acid sequence and a partial peptide having the amino acid sequence leading to loss of expression with respect to each of loss of expression sites of B-Raf and PETN, are respectively used as antigens, antibodies specific to the loss of expression sites can be prepared by a conventional method. In preparing an antibody specific to a loss of expression site of PTEN, the loss of expression site can be detected if no detection is made by the antibody recognizing a wild type. When a mutation or loss of expression is detected, mutation-site specific antibodies may be used alone or in combination of two or more types.

When a mutation and loss of expression are detected by mass spectrometry, detection can be made, for example, by MassARRAY system manufactured by Sequenom in accordance with the method of Gabriel et al., (Gabriel et al., “Current Protocols in Human Genomics”, John Wiley & Sons, 2009, Unit 2.12).

A mutation or loss of expression of B-Raf and PTEN can be detected by any one of the aforementioned methods or in combination of them.

In the above detection, if the result that (a1) B-Raf is wild type and PTEN is wild type, or (a2) B-Raf has at least one mutation selected from Table 1 or loss of expression and PTEN has at least one mutation selected from Table 2 or loss of expression, is obtained, the result serves as an indicator that the responsiveness to the angiogenesis inhibitor in the subject is high. The forms of B-Raf and PTEN, which serve as an indicator that the responsiveness of the subject to the angiogenesis inhibitor is high, are shown in Table 3.

TABLE 3

| | B-Raf | PTEN | Responsiveness |
|---|---|---|---|
| a1 | Wild type | Wild type | High |
| a2 | At least one mutation | At least one mutation | High |
| | selected from Table 1 | selected from Table 2 | |
| | or loss of expression | or loss of expression | |
| | is present | is present | |

In (a2) of Table 2, the forms of B-Raf and PTEN, which serve as an indicator that the responsiveness of the subject to the angiogenesis inhibitor is particularly high, are obtained when the result that B-Raf has a V600E mutation and PTEN has a T167A, Y68H or L112Q mutation, is obtained.

A method for quantifying the expression levels of ANG1 or ANG2 in a sample derived from a tumor tissue of a subject will be described below.

A sample derived from a tumor tissue of a subject is taken by the aforementioned method.

The expression levels of ANG1 or ANG2 in the sample can be obtained by quantifying the amount of mRNA or protein by a conventional method.

In quantifying the amount of mRNA, a conventional technique such as Northern blot analysis, DNA microarray, RT-PCR, and/or quantitative PCR can be used; however, DNA microarray or quantitative PCR are preferably used.

As the probe for use in quantifying the expression levels of ANG1 or ANG2, a nucleic acid probe or antibody against ANG1 or ANG2 is mentioned. The nucleic acid probe can be purchased, for example, through ASSAYS-ON-DEMAND of Applied Biosystems (assay IDs of ANG1 and ANG2 are Hs 00181613 and Hs 00169867, respectively). The expression levels may be quantified in accordance with the manual attached to the probes. Alternatively, the nucleic acid probe can be appropriately set and prepared based on the nucleotide sequence of ANG1 or ANG2 by use of Primer Express of Perkin-Elmer Applied Biosystems or a software equivalent to it

A plurality of test substances are compared by correcting quantitative value based on mRNA level of a house keeping gene (transcription amount is not so much fluctuated), preferably β-actin of each test subject. Note that when mRNA is used for RT-PCR, a primer is used in detecting with a fluorescent dye, SYBR Green (intercalator); whereas, in a detection method using a Master mix, not only a primer but also a probe is required. Either one of them can be designed by use of a software. When a probe is used, a commercially available probe designed by Applied Biosystems may be used.

Furthermore, the expression level of a protein can be determined by a commercially available ELISA kit or by Western blotting, an antibody array, mass spectrometry or immunohistostaining. A specific antibody to ANG1 or ANG2 can be prepared also by the method described in the above paragraph of the angiogenesis inhibitor. When the serum or the plasma is used as a sample, quantification using ELISA and a multiplex beads technique can be used.

The expression level of ANG1 or ANG2 can be quantified by any one of the aforementioned methods or in combination of them.

In the present invention, to quantify the expression level of ANG1 or ANG2, a sample derived from a tumor tissue of a subject is preferably brought into contact with a probe. The meaning of “brought into contact with” is the same as defined above.

In the aforementioned detection, if the result that (b1) the expression level of ANG1 is low compared to a control value; (b2) the expression level of ANG2 is high compared to a control value; or (b3) the ratio of the expression levels of ANG1 and ANG2 is low compared to a control value is obtained, the result serves as an indicator that the responsiveness of the subject to an angiogenesis inhibitor is high. The forms of the expression levels of ANG1 and ANG2, which serve as an indicator that the responsiveness of a subject to an angiogenesis inhibitor is high, are shown in Table 4.

TABLE 4

| | Expression level | Expression level | |
|---|---|---|---|
| | of ANG1 | of ANG2 | Responsiveness |
| b1 | Lower than control | — | High |
| | value | | |
| b2 | — | Higher than control | High |
| | | value | |

| | | |
|---|---|---|
| b3 | Ratio of expression levels of ANG1 and | High |
| | ANG2 is lower than control value | |

In the above b1, herein, the “control” includes a sample obtained in the past. The sample is to be used as a reference for future comparison with a test sample derived from a subject who was predicted to have therapeutic responsiveness. More specifically, the control value means a cutoff value, which is obtained by administering a specific angiogenesis inhibitor to patients who are suffering from the same type of tumor, and analyzing expression levels of ANG1 in patients who are evaluated as being resistant and patients who are evaluated as being sensitive. The cutoff value can be easily determined. For example, a control value may be determined as a cutoff value, which is the expression level of ANG1 in a sample derived from a tumor tissue of patients who have been predicted to have therapeutic responsiveness by administration of an angiogenesis inhibitor.

Alternatively, the control value may mean a cutoff value determined based on the presence or absence of a mutation or loss of expression in B-Raf and PTEN. In this case, the control value refers to the expression level of ANG1 in a patient suffering from a tumor having wild-type B-Raf. In this case, a preferable control value is the expression level of ANG1 in a patient suffering from a tumor having wild-type B-Raf and wild-type PTEN.

In this case, the form of expression level of ANG1, which serves as an indicator that the responsiveness of a subject to the angiogenesis inhibitor is high, is obtained when the result of being equal to or less than the expression level of ANG1 observed in a patient suffering from a tumor having wild-type B-Raf, and particularly preferably, being equal to or less than the expression level of ANG1 observed in a patient suffering from a tumor having wild-type B-Raf and wild type PTEN, is obtained.

In the above b2, the “control” includes a sample obtained in the past. The sample is to be used as a reference for future comparison with a test sample derived from a subject who was predicted to have therapeutic responsiveness. More specifically, the control value means a cutoff value, which is obtained by administering a specific angiogenesis inhibitor to patients who are suffering from the same type of tumor and analyzing expression levels of ANG2 in patients who are evaluated as being resistant and patients who are evaluated as being sensitive. The cutoff value can be easily determined. For example, a control value may be determined as a cutoff value, which is the expression level of ANG2 in a sample derived from a tumor tissue of patients who have been predicted to have therapeutic responsiveness by administration of an angiogenesis inhibitor.

Alternatively, the control value may mean a cutoff value determined based on expression levels of ANG1 and ANG2. In this case, the phrase that the expression level of ANG2 is higher than a control value means that the expression level of ANG2 in terms of absolute value is higher than the expression level of ANG1. In this case, a preferable control value refers to the expression levels of ANG1 and ANG2 in a patient suffering from a tumor in which B-Raf has at least one mutation from Table 1 or loss of expression selected and PTEN has at least one mutation selected from Table 2 or loss of expression.

In this case, particularly, the form of expression level of ANG2, which serves as an indicator that the responsiveness of a subject to the angiogenesis inhibitor is high, is obtained when the result of being higher than the expression level of ANG1 observed in a patient suffering from a tumor in which B-Raf has at least one mutation selected from Table 1 or loss of expression and PTEN has at least one mutation selected from Table 2 or loss of expression, is obtained.

The expression levels of ANG1 and ANG2 in a patient suffering from a tumor in which B-Raf has at least one mutation selected from Table 1 or loss of expression and PTEN has at least one mutation selected from Table 2 or loss of expression are preferably the expression levels of ANG1 and ANG2 in a patient suffering from a tumor in which B-Raf has V600E mutation and PTEN has T167A, Y68H or L112Q mutation.

In the above b3, the “control” includes a sample obtained in the past. The sample is to be used as a reference for future comparison with a test sample derived from a subject who was predicted to have therapeutic responsiveness. More specifically, the control value means a cutoff value, which is obtained by administering a specific angiogenesis inhibitor to patients who are suffering from the same type of tumor and analyzing the ratio of expression levels of ANG1 and ANG2 in patients who are evaluated as being resistant and patients who are evaluated as being sensitive. The cutoff value can be easily determined. For example, a control value may be determined as a cutoff value, which is the ratio of expression levels of ANG1 and ANG2 (ANG1 expression level/ANG2 expression level) in a sample derived from a tumor tissue of patients who have been predicted to have therapeutic responsiveness by administration of an angiogenesis inhibitor.

Alternatively, the control value may be a cutoff value, which is determined based on the presence or absence of a mutation or loss of expression in B-Raf and PTEN in place of the analysis of the ratio of expression levels of ANG1 and ANG2.

In this case, the preferable control value is a cutoff value, which is the ratio of expression levels of ANG1 and ANG2 in patients in which B-Raf has a mutation or loss of expression and PTEN is a wild type, preferably, the ratio of expression levels of ANG1 and ANG2 in a patient suffering from a tumor in which B-Raf has at least one mutation selected from Table 1 or loss of expression and PTEN is wild type, and more preferably, the ratio of expression levels of ANG1 and ANG2 in a patient suffering from a tumor in which B-Raf has, for example, V600E mutation or A145V mutation and PTEN is wild type.

In this case, particularly, the form of the ratio of expression levels of ANG1 and ANG2, which serves as an indicator that the responsiveness of a subject to the angiogenesis inhibitor is high, is obtained when the result of 1) being equal to or less than the ratio of expression levels of ANG1 and ANG2 in a patient suffering from a tumor in which B-Raf is wild type and PTEN is wild type; 2) bring equal to or less than the ratio of expression levels of ANG1 and ANG2 in a patient suffering from a tumor in which B-Raf has at least one mutation selected from Table 1 or loss of expression and PTEN has at least one mutation selected from Table 2 or loss of expression; or 3) being lower than the ratio of expression levels of ANG1 and ANG2 in a patient suffering from a tumor in which B-Raf has at least one mutation selected from Table 1 or loss of expression and PTEN is wild type, is obtained.

A preferable aspect of the ratio of expression levels of ANG1 and ANG2 in a patient suffering from a tumor in which B-Raf has at least one mutation selected from Table 1 or loss of expression and PTEN has at least one mutation selected from Table 2 or loss of expression is the ratio of expression levels of ANG1 and ANG2 in a patient suffering from a tumor in which B-Raf has V600E mutation and PTEN has T167A, Y68H or L112Q mutation. A preferable aspect of the ratio of expression levels of ANG1 and ANG2 in a patient suffering from a tumor in which B-Raf has at least one mutation selected from Table 1 or loss of expression and PTEN is wild type is the ratio of expression levels of ANG1 and ANG2 in a patient suffering from a tumor in which B-Raf has V600E or A145V mutation and PTEN is wild type.

A method for quantifying the expression levels of SHC1, IL6, CXCR4, COL4A3, NRP2, MEIS1, ARHGAP22, SCG2, FGF9, PML, FGFR3, FGFR2, FGFR1, FGFR4 and VEGFR1 in a sample derived from a tumor tissue of a subject will be described below.

A method of quantifying the expression levels of SHC1, IL6, CXCR4, COL4A3, NRP2, MEIS1, ARHGAP22, SCG2, FGF9, PML, FGFR3, FGFR2, FGFR1, FGFR4 and VEGFR1 in a sample derived from a tumor tissue of a subject is the same as in the aforementioned method for quantifying the expression levels of ANG1 and ANG2 in a sample derived from a tumor tissue of a subject except that an object to be quantified is changed to mRNA or protein of SHC1, IL6, CXCR4, COL4A3, NRP2, MEIS1, ARHGAP22, SCG2, FGF9, PML, FGFR3, FGFR2, FGFR1, FGFR4 or VEGFR1.

Alternatively, the expression level of SHC1, IL6, CXCR4, COL4A3, NRP2, MEIS1, ARHGAP22, SCG2, FGF9, PML, FGFR3, FGFR2, FGFR1, FGFR4 or VEGFR1 in a sample derived from a tumor tissue of a subject can be quantified by analyzing gene expression by a DNA microarray.

In the aforementioned detection, as the probe for quantifying the expression level of SHC1, NRP2, ARHGAP22, SCG2, PML, IL6, CXCR4, COL4A3, MEIS1, FGF9, FGFR3, FGFR2, FGFR1, FGFR4 or VEGFR1, a commercially available product (for example, if a nucleic acid probe is used, it can be purchased through ASSAYS-ON-DEMAND of Applied Biosystems) can be appropriately used.

In the aforementioned detection, since the expression level of SHC1, NRP2, ARHGAP22, SCG2, PML, IL6, CXCR4, COL4A3, MEIS1, FGF9, FGFR3, FGFR2, FGFR1, FGFR4 or VEGFR1 is correlated with the presence or absence of a mutation or loss of expression in B-Raf and PTEN, the expression levels of these genes are correlated with anti-tumor effect determined by the presence or absence of a mutation or loss of expression in B-Raf and PTEN. The expression levels of SHC1, NRP2, ARHGAP22, SCG2 and PML each exhibit the same behavior as the fluctuation pattern of the anti-tumor effect determined by the presence or absence of a mutation or loss of expression in B-Raf and PTEN; whereas the expression levels of IL6, CXCR4, COL4A3, MEIS1, FGF9, FGFR3, FGFR2, FGFR1, FGFR4 and VEGFR1 each exhibit opposite behavior to the fluctuation pattern of the anti-tumor effect determined by the presence or absence of a mutation or loss of expression in B-Raf and PTEN.

Therefore, in the case where the result: (c1) the expression level of SHC1 is low compared to a control value, (c2) the expression level of NRP2 is low compared to a control value, (c3) the expression level of ARHGAP22 is low compared to a control value, (c4) the expression level of SCG2 is low compared to a control value, (c5) the expression level of PML is low compared to a control value, (c6) the expression level of IL6 is high compared to a control value, (c7) the expression level of CXCR4 is high compared to a control value, (c8) the expression level of COL4A3 is high compared to a control value, (c9) the expression level of MEIS1 is high compared to a control value, (c10) the expression level of FGF9 is high compared to a control value, (c11) the expression level of FGFR3 is high compared to a control value, (c12) the expression level of FGFR2 is high compared to a control value, (c13) the expression level of FGFR1 is high compared to a control value, (c14) the expression level of FGFR4 is high compared to a control value, or (c15) the expression level of VEGFR1 is high compared to a control value is obtained, the result serves as an indicator that the responsiveness of a subject to the angiogenesis inhibitor is high. The responsiveness to an angiogenesis inhibitor can be predicted by analyzing one or a plurality of the cases selected from (c1) to (c15) in combination.

In the above (c1) to (c15), the control value refers to the expression level of SHC1, NRP2, ARHGAP22, SCG2, PML, IL6, CXCR4, COL4A3, MEIS1, FGF9, FGFR3, FGFR2, FGFR1, FGFR4 or VEGFR1 in a patient suffering from a tumor in which B-Raf has at least one mutation selected from Table 1 or loss of expression and PTEN is wild type.

In the above (c1) to (c5), particularly, the forms of the expression levels of SHC1, NRP2, ARHGAP22, SCG2 and PML, which serve as an indicator that the responsiveness of a subject to the angiogenesis inhibitor is high, are obtained when the result of being low compared to the expression levels of SHC1, NRP2, ARHGAP22, SCG2 and PML in a patient suffering from a tumor in which B-Raf has at least one mutation selected from Table 1 or loss of expression and PTEN is wild type, is obtained.

In this case, a preferable aspect of each of the expression levels of SHC1, NRP2, ARHGAP22, SCG2 and PML in a patient suffering from a tumor in which B-Raf has at least one mutation selected from Table 1 or loss of expression and PTEN is wild type is the expression level of each of SHC1, NRP2, ARHGAP22, SCG2 and PML in a patient suffering from a tumor in which B-Raf has V600E or A145V mutation and PTEN is wild type.

In the above (c6) to (c15), particularly, the forms of the expression levels of IL6, CXCR4, COL4A3, MEIS1, FGF9, FGFR3, FGFR2, FGFR1, FGFR4 and VEGFR1, which serve as an indicator that the responsiveness of a subject to the angiogenesis inhibitor is high, are obtained when the result of being high compared to the expression levels of IL6, CXCR4, COL4A3, MEIS1, FGF9, FGFR3, FGFR2, FGFR1, FGFR4 and VEGFR1 in a patient suffering from a tumor in which B-Raf has at least one mutation selected from Table 1 or loss of expression and PTEN is wild type, is obtained.

In this case, a preferable aspect of each of the expression levels of IL6, CXCR4, COL4A3, MEIS1, FGF9, FGFR3, FGFR2, FGFR1, FGFR4 and VEGFR1 in a patient suffering from a tumor in which B-Raf has at least one mutation selected from Table 1 or loss of expression and PTEN is wild is the expression level of each of IL6, CXCR4, COL4A3, MEIS 1, FGF9, FGFR3, FGFR2, FGFR1, FGFR4 and VEGFR1 in a patient suffering from a tumor in which B-Raf has V600E or A145V mutation and PTEN is wild type.

The forms of the expression levels of SHC1, NRP2, ARHGAP22, SCG2, PML, IL6, CXCR4, COL4A3, MEIS1, FGF9, FGFR3, FGFR2, FGFR1, FGFR4 and VEGFR1, which serve as an indicator that the responsiveness of a subject to the angiogenesis inhibitor is high, are shown in Table 5.

TABLE 5

| | Target to be | | |
|---|---|---|---|
| | quantified | Expression level | Responsiveness |
| c1 | SHC1 | Lower than control value | High |
| c2 | NRP2 | Lower than control value | High |
| c3 | ARHGAP22 | Lower than control value | High |
| c4 | SCG2 | Lower than control value | High |
| c5 | PML | Lower than control value | High |
| c6 | IL6 | Higher than control value | High |
| c7 | CXCR4 | Higher than control value | High |
| c8 | COL4A3 | Higher than control value | High |
| c9 | MEIS1 | Higher than control value | High |
| c10 | FGF9 | Higher than control value | High |
| c11 | FGFR3 | Higher than control value | High |
| c12 | FGFR2 | Higher than control value | High |
| c13 | FGFR1 | Higher than control value | High |
| c14 | FGFR4 | Higher than control value | High |
| c15 | VEGFR1 | Higher than control value | High |

Another aspect of the present invention is the case where the determination result of the following determination target:

(a) B-Raf and PTEN,

(b) ANG1 and ANG2, or

(c) at least one selected from the group consisting of SHC1, NRP2, ARHGAP22, SCG2, PML, IL6, CXCR4, COL4A3, MEIS1, FGF9, FGFR3, FGFR2, FGFR1, FGFR4 and VEGFR1

determined in a sample taken form a single patient is compared to the control value of each of the targets to thereby associate the responsiveness to an angiogenesis inhibitor, and, in addition, the aforementioned determination targets are quantified or detected in samples derived from a plurality of patients. Accordingly, the presence or absence of mutation or the expression level of each of the aforementioned determination targets is detected or determined in the predetermined number of patients (primary population) and the obtained detection value or measurement value, which is used as basic data, can be compared to measurement data in the sample derived from a single subject or samples derived from a plurality of populations (secondary population).

Alternatively, the measurement data of individual patients are added to values of the primary population and the entire data are processed again. In this manner, the number of target patients or cases of the secondary population can be increased. The prediction accuracy for responsiveness to an angiogenesis inhibitor can be enhanced by increasing the number of cases.

The method according to the present invention can be used for predicting the level of efficacy of an angiogenesis inhibitor in a subject before the angiogenesis inhibitor is administered to the subject. In this way, a subject in which a higher effect of an angiogenesis inhibitor can be expected is selected to treat a disease. As the case where a higher anti-tumor effect can be expected, a case where a higher anti-tumor effect can be expected than an average anti-tumor effect in subjects presenting similar symptoms; a case where a higher anti-tumor effect can be expected than those in other subjects suffering from the same type of cancer; or a case where a higher anti-tumor effect can be expected than that of a subject suffering from another type of cancer, can be mentioned. Therefore, the present invention is clinically very useful.

As another aspect of the present invention, there is provided a method of using data, in administering an angiogenesis inhibitor to a subject suffering from a tumor or in treating the tumor, based on

(a) the presence or absence of a mutation or loss of expression in B-Raf and PTEN,

(b) the expression levels of ANG1 and ANG2, or

(c) the expression level of at least one selected from the group consisting of SHC1, NRP2, ARHGAP22, SCG2, PML, IL6, CXCR4, COL4A3, MEIS1, FGF9, FGFR3 and FGFR2. As described above, a case where (a1) B-Raf is wild type and PTEN is wild type, (a2) B-Raf has at least one mutation selected from Table 1 or loss of expression and PTEN has at least one mutation selected from Table 2 or loss of expression, (b1) the expression level of ANG1 is low compared to a control value (b2) the expression level of ANG1 is equal to and higher compared to a control value and the expression level of ANG2 is sufficient to cancel out the expression of ANG1 (b3) the ratio of the expression levels of ANG1 and ANG2 is low compared to a control value (c1) the expression level of SHC1 is low compared to a control value (c2) the expression level of NRP2 is low compared to a control value (c3) the expression level of ARHGAP22 is low compared to a control value (c4) the expression level of SCG2 is low compared to a control value (c5) the expression level of PML is low compared to a control value (c6) the expression level of IL6 is high compared to a control value (c7) the expression level of CXCR4 is high compared to a control value (c8) the expression level of COL4A3 is high compared to a control value (c9) the expression level of MEIS1 is high compared to a control value (c10) the expression level of FGF9 is high compared to a control value (c11) the expression level of FGFR3 is high compared to a control value (c12) the expression level of FGFR2 is high compared to a control value (c13) the expression level of FGFR1 is high compared to a control value (c14) the expression level of FGFR4 is high compared to a control value or (c15) the expression level of VEGFR1 is high compared to a control value serves as an indicator that it is effective to administer an angiogenesis inhibitor to the subject or treat a tumor. Use of the indicator enables to evaluate how highly a subject responds to an angiogenesis inhibitor and evaluate the possibility of a subject to respond to an angiogenesis inhibitor. The evaluation results are useful data as a reference in determining the right or wrong of administration of an angiogenesis inhibitor to a subject or in selecting e.g., a tumor therapeutic regimen using an angiogenesis inhibitor. However, in the present invention, since an angiogenesis inhibitor basically has an inhibitory action on angiogenesis, even if a subject is determined not to be highly sensitive to an angiogenesis inhibitor, it is not predicted that the angiogenesis inhibitor has no anti-tumor effect.

Note that a person who administers an angiogenesis inhibitor to a subject suffering from a tumor or a person who treats a tumor and a person who performs the measurement of the above (a1) to (c15) may be the same or different.

Another aspect of the present invention, there is provided a method for administering an angiogenesis inhibitor to a subject suffering from a tumor or treating the tumor by using

(a) the presence or absence of a mutation or loss of expression in B-Raf and PTEN,

(b) the expression levels of ANG1 and ANG2, or

(c) the expression level of at least one selected from the group consisting of SHC1, IL6, CXCR4, COL4A3, NRP2, MEIS1, ARHGAP22, SCG2, FGF9, PML, FGFR3, FGFR2, FGFR1, FGFR4 and VEGFR1, as an indicator. As described above, the case where (a1) B-Raf is wild type and PTEN is wild type; (a2) B-Raf has at least one mutation selected from Table 1 or loss of expression and PTEN has at least one mutation selected from Table 2 or loss of expression; (b1) the expression level of ANG1 is low compared to a control value; (b2) the expression level of ANG1 is equal to and higher compared to a control value and the expression level of ANG2 is sufficient to cancel out the expression of ANG1; (b3) the ratio of the expression levels of ANG1 and ANG2 is low compared to a control value; (c1) the expression level of SHC1 is low compared to a control value; (c2) the expression level of NRP2 is low compared to a control value; (c3) the expression level of ARHGAP22 is low compared to a control value; (c4) the expression level of SCG2 is low compared to a control value; (c5) the expression level of PML is low compared to a control value; (c6) the expression level of IL6 is high compared to a control value; (c7) the expression level of CXCR4 is high compared to a control value; (c8) the expression level of COL4A3 is high compared to a control value; (c9) the expression level of MEIS 1 is high compared to a control value; (c10) the expression level of FGF9 is high compared to a control value; (c11) the expression level of FGFR3 is high compared to a control value; (c12) the expression level of FGFR2 is high compared to a control value; (c13) the expression level of FGFR1 is high compared to a control value; (c14) the expression level of FGFR4 is high compared to a control value; or (c15) the expression level of VEGFR1 is high compared to a control value, is an indicator that it is effective to administer the angiogenesis inhibitor to the subject and treat a tumor. In determining right or wrong of administration of an angiogenesis inhibitor to a subject or selecting e.g., a tumor therapeutic regimen using an angiogenesis inhibitor, the subject, to whom administration of the angiogenesis inhibitor or treatment of the tumor is predicted to be effective, can be selected as an administration target for the angiogenesis inhibitor. Therefore, the present invention encompasses a method for treating a subject, who is suffering from a tumor and predicted to be highly responsive to an angiogenesis inhibitor by the prediction method of the present invention, by administering the angiogenesis inhibitor. However, in the present invention, since an angiogenesis inhibitor basically has an inhibitory action on angiogenesis, even if a subject is determined not to be highly sensitive to an angiogenesis inhibitor, it is not predicted that the angiogenesis inhibitor has no anti-tumor effect.

Note that a person who administers an angiogenesis inhibitor to a subject suffering from a tumor or a person who treats a tumor and a person who performs the measurement of the above (a1) to (c15) may be the same or different.

As another aspect of the present invention, there is provided a method for selecting a subject who is highly sensitive to an angiogenesis inhibitor by using

(a) the presence or absence of a mutation or loss of expression in B-Raf and PTEN,

(b) the expression levels of ANG1 and ANG2, or

(c) the expression level of at least one selected from the group consisting of SHC1, IL6, CXCR4, COL4A3, NRP2, MEIS1, ARHGAP22, SCG2, FGF9, PML, FGFR3, FGFR2, FGFR1, FGFR4 and VEGFR1, as an indicator. As described above, the case where (a1) B-Raf is wild type and PTEN is wild type; (a2) B-Raf has at least one mutation selected from Table 1 or loss of expression and PTEN has at least one mutation selected from Table 2 or loss of expression; (b1) the expression level of ANG1 is low compared to a control value; (b2) the expression level of ANG1 is equal to and higher compared to a control value and the expression level of ANG2 is sufficient to cancel out the expression of ANG1; (b3) the ratio of the expression levels of ANG1 and ANG2 is low compared to a control value; (c1) the expression level of SHC1 is low compared to a control value, (c2) the expression level of NRP2 is low compared to a control value; (c3) the expression level of ARHGAP22 is low compared to a control value; (c4) the expression level of SCG2 is low compared to a control value; (c5) the expression level of PML is low compared to a control value; (c6) the expression level of IL6 is high compared to a control value; (c7) the expression level of CXCR4 is high compared to a control value; (c8) the expression level of COL4A3 is high compared to a control value; (c9) the expression level of MEIS1 is high compared to a control value; (c10) the expression level of FGF9 is high compared to a control value; (c11) the expression level of FGFR3 is high compared to a control value; (c12) the expression level of FGFR2 is high compared to a control value; (c13) the expression level of FGFR1 is high compared to a control value; (c14) the expression level of FGF′R4 is high compared to a control value; or (c15) the expression level of VEGFR1 is high compared to a control value, is an indicator that the subject is highly sensitive to an angiogenesis inhibitor. Accordingly, such a subject can be selected as the subject who is highly sensitive to the angiogenesis inhibitor.

Note that a person who selects a subject who is highly sensitive to the angiogenesis inhibitor and a person who performs the measurement of the above (a1) to (c15) may be the same or different.

As another aspect of the present invention, there is provided a pharmaceutical composition comprising an angiogenesis inhibitor. A subject to which the pharmaceutical composition of the present invention is to be administered is a subject, who is suffering from a tumor and has been predicted to be highly responsive to the angiogenesis inhibitor by the method of the present invention. Furthermore, the present invention provides use of an angiogenesis inhibitor for producing a medicinal drug to be administered to a subject suffering from a tumor. The subject is a subject who has been predicted to be highly responsive to the angiogenesis inhibitor by the method of the present invention. Furthermore, the present invention provides an angiogenesis inhibitor for treating a subject suffering from a tumor and the subject is a subject who has been predicted to be highly responsive to the angiogenesis inhibitor by the method of the present invention.

In the pharmaceutical composition and tumor therapy method of the present invention targeting tumor cells having a mutation or loss of expression of B-Raf and PTEN, one or a plurality of other anti-tumor agents may be used in combination. The other anti-tumor agent is not particularly limited as long as it is a preparation having an anti-cancer activity. Examples of the other anti-tumor agent include irinotecan hydrochloride (CPT-11), carboplatin, oxaliplatin, 5-fluorouracil (5-FU), docetaxel (Taxotere (registered trade mark)), paclitaxel, gemcitabine hydrochloride (Gemzar (registered trade mark)), calcium folinate (Leucovorin), bevacizumab (Avastin (registered trade mark)) and everolimus (Certican (registered trade mark) or Afinitor (registered trade mark)). Furthermore, examples of the other anti-tumor agent particularly preferably include dacarbazine or temozolomide when the kind of tumor to be treated by a tumor therapeutic agent is melanoma; irinotecan hydrochloride, oxaliplatin, 5-fluorouracyl, calcium folinate or bevacizumab when it is large bowel cancer; gemcitabine hydrochloride or bevacizumab when it is pancreatic cancer; carboplatin or gemcitabine hydrochloride when it is ovarian cancer, bevacizumab or everolimus when it is kidney cancer; and carboplatin, docetaxel or paclitaxel when it is lung cancer.

The pharmaceutical composition of the present invention can be used as a tumor therapeutic agent. In the present invention, the tumor therapeutic agent includes an anti-tumor agent, a cancer prognosis improving agent, a cancer recurrence preventive and a cancer metastasis inhibitor.

The effect of cancer therapy can be confirmed by observation such as radiograph, CT and histopathological diagnosis such as biopsy or a value of a tumor marker.

When the pharmaceutical composition of the present invention is used, it can be orally or parenterally administered. In using the pharmaceutical composition of the present invention, the dose of an angiogenesis inhibitor varies depending upon e.g., severity of symptom, the age, sex, weight and degree of sensitiveness of a subject, an administration route, an dosing timing, an dosing interval, properties, formulation and type of a pharmaceutical formulation and type of active ingredient. Although it is not particularly limited; the dose is usually 0.1 mg to 10 g per adult (body weight: 60 kg) per day, which is divided into portions and administered at a frequency of usually from one per week to three times per day.

The pharmaceutical composition of the present invention can be formulated into e.g., an oral solid formulation and an injection. Examples of the oral solid formulation include a tablet, a coated tablet, a granule, a fine grain formulation, a powder and an encapsulated formulation. Examples of the injection include an intravenous injection, a subcutaneous injection and an intramuscular injection. If necessary, they can be lyophilized by a conventional method.

In formulating into a formulation, additives conventionally used such as an excipient, a binding agent, a lubricant, a colorant and a flavoring agent can be used and, if necessary, a stabilizer, an emulsifier, an absorption accelerator, a surfactant and others can be used. Generally, components used as raw materials for a pharmaceutical formulation are blended and formulated into a formulation in accordance with a conventional method.

Examples of these components include animal and vegetable oils (e.g., soybean oil, beef tallow, synthetic glyceride), hydrocarbon (e.g., fluid paraffin, squalane, solid paraffin), ester oils (e.g., octyldodecyl myristate, isopropyl myristate), higher alcohols (e.g., cetostearyl alcohol, behenyl alcohol), silicon resin, silicon oil, surfactants (e.g., polyoxyethylene fatty acid ester, sorbitan fatty acid ester, glycerin fatty acid ester, polyoxyethylene sorbitan fatty acid ester, polyoxyethylene hydrogenated castor oil, polyoxyethylene-polyoxypropylene block copolymer), water soluble polymers (e.g., hydroxyethylcellulose, polyacrylic acid, carboxyvinyl polymer polyethylene glycol, polyvinyl pyrrolidone, methylcellulose), alcohols (e.g., ethanol, isopropanol), polyhydric alcohols (e.g., glycerin, propylene glycol, dipropylene glycol, sorbitol), sugars (e.g., glucose, sucrose), inorganic powders (silicic anhydride, magnesium aluminum silicate, aluminum silicate) and purified water. For controlling pH, e.g., an inorganic acid (e.g., hydrochloric acid, phosphoric acid), an alkaline metal salt of an inorganic acid (e.g., sodium phosphate), an inorganic base (e.g., sodium hydroxide), an organic acid (e.g., lower fatty acid, citric acid, lactic acid), an alkali metal salt of an organic acid (e.g., sodium citrate, sodium lactate) and/or an organic base (e.g., arginine, ethanolamine) can be used. If necessary, an antiseptic agent and/or an antioxidant can be added.

As another aspect of the present invention, the present invention provides a kit for predicting responsiveness of a subject suffering from a tumor to an angiogenesis inhibitor, characterized by comprising probes of B-Raf and PTEN or ANG1 and ANG2. Prediction of the responsiveness to an angiogenesis inhibitor can be performed by the method of the present invention. As the angiogenesis inhibitor, preferably, 4-(3-chloro-4-(cyclopropylaminocarbonyl)aminophenoxy)-7-methoxy-6-quinolinecarboxamide or a pharmacologically acceptable salt thereof is mentioned.

The pharmaceutical composition of the present invention and/or kit can be applied to mammals (for example, a human, a rat, a rabbit, a sheep, a pig, a cow, a cat, a dog, a monkey).

The pharmaceutical composition of the present invention and/or kit may comprise, other than the angiogenesis inhibitor or probes, e.g., a package container, an instruction booklet and a package insert. In the package container, instruction booklet and package insert, for example, combination of one or a plurality of other anticancer agents concomitantly used can be described. Furthermore, dosage and administration with respect to the form for administering independent substances in combination or a form as a mixture. The dosage and administration can be described, with reference to the above.

### Examples

The present invention will be more specifically described by way of Examples; however, the present invention is not limited to these examples.

### Example 1

### Detection of Mutation or Loss of Expression of BRAF and PTEN

Human melanoma cell lines, SK-MEL-2, MeWo, CHL-1, HMV-1, HMCB, MDA-MB-435, LOX, G361, FEM, SEKI, SK-MEL-28, A375 and A2058 were each obtained from the manufacturers shown in the column of “distributor” of Table 6 and analyzed by the Sanger method or a next generation sequence method (Bridge PCR method: Solexa/Illumina) to detect a mutation or loss of expression of BRAF and PTEN.

(1) Detection by the Sanger Method

(i) Preparation of genomic DNA from melanoma cell line

Genomic DNA was purified from cells (about 1×10⁶) by use of DNeasy Blood & Tissue Kit (purchased from QIAGEN).

(ii) Amplification of PTEN exon region

The obtained genomic DNA was subjected to PCR to amplify the exon region of PTEN. PCR was performed by PrimeSTAR GXL DNA Polymerase (purchased from Takara Bio Inc.). Genomic DNA (100 ng), 5× PrimeSTAR GXL Buffer (4 μL), a dNTP mixture (2.5 mM) (1.6 μL), a sense primer and an anti-sense primer (5 pmol for each) and PrimeSTAR GXL DNA Polymerase (0.4 μl) were mixed to prepare a solution having a total volume of 20 μL. The solution was subjected to a reaction which was performed by repeating, 40 times, a cycle consisting of a reaction at 95° C. for 10 seconds, a reaction at 55° C. for 15 seconds and a reaction at 68° C. for 30 seconds.

The sequences of primers used in the PCR are shown below.

| | |
|---|---|
| Exon1 Sense Primer: | |
| AGTCGCCTGTCACCATTTC | (SEQ ID NO: 19) |
| Exon1 Antisense Primer: | |
| ACTACGGACATTTTCGCATC | (SEQ ID NO: 20) |
| Exon2 Sense Primer: | |
| GTTTGATTGCTGCATATTTCAG | (SEQ ID NO: 21) |
| Exon2 Antisense Primer: | |
| GGCTTAGAAATCTTTTCTAAATG | (SEQ ID NO: 22) |
| Exon3 Sense Primer: | |
| AATGACATGATTACTACTCTA | (SEQ ID NO: 23) |
| Exon3 Antisense Primer: | |
| TTAATCGGTTTAGGAATACAA | (SEQ ID NO: 24) |
| Exon4 Sense Primer: | |
| CATTATAAAGATTCAGGCAATG | (SEQ ID NO: 25) |
| Exon4 Antisense Primer: | |
| GACAGTAAGATACAGTCTATC | (SEQ ID NO: 26) |
| Exon5 Sense Primer: | |
| ACCTGTTAAGTTTGTATGCAAC | (SEQ ID NO: 27) |
| Exon5 Antisense Primer: | |
| TCCAGGAAGAGGAAAGGAAA | (SEQ ID NO: 28) |
| Exon6 Sense Primer: | |
| CATAGCAATTTAGTGAAATAACT | (SEQ ID NO: 29) |
| Exon6 Antisense Primer: | |
| GATATGGTTAAGAAAACTGTTC | (SEQ ID NO: 30) |
| Exon7 Sense Primer: | |
| TGACAGTTTGACAGTTAAAGG | (SEQ ID NO: 31) |
| Exon7 Antisense Primer: | |
| GGATATTTCTCCCAATGAAAG | (SEQ ID NO: 32) |
| Exon8 Sense Primer: | |
| CTCAGATTGCCTTATAATAGT | (SEQ ID NO: 33) |
| Exon8 Antisense Primer: | |
| TCATGTTACTGCTACGTAAAC | (SEQ ID NO: 34) |
| Exon9 Sense Primer: | |
| AAGGCCTCTTAAAGATCATG | (SEQ ID NO: 35) |
| Exon9 Antisense Primer: | |
| TTTTCATGGTGTTTTATCCCT | (SEQ ID NO: 36) |

(iii) Recovery of PCR Product

The PCR product, which was confirmed to have a desired length by 1% agarose gel electrophoresis, was recovered from the gel and purified by use of Wizard SV Gel and PCR Clean-Up System (purchased from Promega). The purified genomic DNA was subjected to PCR by a commercially available kit to determine the sequence.

(2) Detection by the Next Generation Sequence Method

(i) Preparation of Total RNA from Melanoma 13 Cell Line

Cells were cultured in a 5% CO₂ condition at 37° C. After a predetermined period of time, the cells were lysed with TRIZOL reagent (purchased from GIBCO BRL) in accordance with the operation manual described in the attachment of the reagent.

The method was more specifically performed as follows. TRIZOL reagent was added at a ratio of 1 ml per culture area (10 cm²), pipetted several times and then the liquid containing cell lysis was recovered. The sample thus recovered was centrifuged and the resultant supernatant was allowed to stand at room temperature for 5 minutes. To the sample, chloroform (purchased from Junsei Chemical Co., Ltd.) was added at a ratio of 0.2 ml to the volume of TRIZO reagent (1 ml). This solution was vigorously shaken and stirred for 15 seconds and stirred, allowed to stand at room temperature for 2 to 3 minutes and centrifuged (12000×g, 10 minutes, 4° C.). After centrifugation, an aqueous layer was transferred to a new tube. To this, isopropyl alcohol (purchased from Wako Pure Chemical Industries Ltd.) was added in a ratio of 0.5 ml to TRIZO reagent (1 ml). The mixture was allowed to stand at room temperature for 10 minutes and then centrifuged (12000×g, 10 minutes, 4° C.). Precipitation was obtained, washed with 75% ethanol (purchased from Wako Pure Chemical Industries Ltd.) and dried in air to obtain total RNA, which was subjected to the following operations.

(ii) Amplification of Sequences Encoding BRAF and PTEN Protein

Using the RNA obtained above as a template, cDNA was synthesized in accordance with the method described in the package insert of High capacity cDNA Reverse Transcription kit.

The resultant cDNAs of melanoma 13 cell lines were subjected to PCR to amplify sequences encoding B-Raf and PTEN proteins. PCR was performed with PrimeSTAR GXL DNA Polymerase (purchased from Takara Bio Inc.) or Phusion High-Fidelity DNA Polymerase (purchased from Finnzymes). In the case of using PrimeSTAR GXL DNA Polymerase, cDNA (100 ng), 5× PrimeSTAR GXL Buffer (4 μL), a dNTP mixture (2.5 mM)(1.6 μL), a sense primer and an anti-sense primer (5 pmol for each) (with respect to B-Raf, a reaction was performed by using three types of primers for each) and PrimeSTAR GXL DNA Polymerase (0.4 μl) were mixed to prepare a solution of a total volume of 20 μL and the solution was subjected to a reaction which was performed by repeating, 40 times, a cycle consisting of a reaction at 95° C. for 10 seconds, a reaction at 55° C. for 15 seconds and a reaction at 68° C. for 2 minutes. In the case of using Phusion High-Fidelity DNA Polymerase, cDNA (100 ng), 5× Phusion GC Buffer (4 μL), a dNTP mixture (2.5 mM) (1.6 μL), a sense primer and an anti-sense primer (10 pmol for each) and Phusion High-Fidelity DNA Polymerase (0.2 μL) were mixed to prepare a solution having a total volume of 20 μL and the solution was subjected to a reaction which was performed by repeating, 40 times, a cycle consisting of a reaction at 98° C. for 10 seconds, a reaction at 55° C. for 30 seconds and a reaction at 72° C. for 2 minutes.

The sequences of the primers used in PCR are shown below.

| | |
|---|---|
| PTEN Sense Primer: | |
| TCTGCCATCTCTCTCCTCCTTTT | (SEQ ID NO: 37) |
| PTEN Antisense Primer: | |
| TCTGACACAATGTCCTATTGCCAT | (SEQ ID NO: 38) |
| BRAF Sense Primer 1: | |
| GCCCCGGCTCTCGGTTATAAGATG | (SEQ ID NO: 39) |
| BRAF Antisense Primer 1: | |
| CCGTTCCCCAGAGATTCCAA | (SEQ ID NO: 40) |
| BRAF Sense Primer 2: | |
| TGCCATTCCGGAGGAGGTGT | (SEQ ID NO: 41) |
| BRAF Antisense Primer 2: | |
| GCCCATCAGGAATCTCCCAA | (SEQ ID NO: 42) |
| BRAF Sense Primer 3: | |
| ATCTGGATCATCCCCTTCCGC | (SEQ ID NO: 43) |
| BRAF Antisense Primer 3: | |
| CCCGGAACAGAAAGTAAAGCCTCTAG | (SEQ ID NO: 44) |

(iii) Recovery, Purification, Blunting and Ligation of PCR Product

The PCR product, which was confirmed to have a desired length by 1% agarose gel electrophoresis, was recovered from the gel and purified by use of Wizard SV Gel and PCR Clean-Up System (purchased from Promega). The purified PCR products were collected to a single tube per cell line and a total volume (10 μL) was subjected to a blunting treatment by a DNA Blunting Kit (purchased from Takara Bio Inc.). Thereafter, phenol/chloroform extraction and ethanol precipitation were performed to obtain DNA pellets and a ligation treatment was performed by use of DNA Ligation Kit (purchased from Takara Bio Inc.) in a total volume of 10 μL at 16° C. for 6 hours. The DNA ligated was subjected to phenol/chloroform extraction and ethanol precipitation to obtain DNA pellets.

(iv) Preparation of Library for Analysis by a Next Generation Sequencer

A library was prepared by use of Genomic DNA Sample Prep Kit (purchased from Illumina) in accordance with the operation manual attached. The outline of the method is as follows.

DNA was subjected to nebulization to obtain fragments. The DNA fragments are blunted and 5′ terminals thereof were phosphorylated. After an adaptor was added, a 2% agarose gel electrophoresis was performed. A product of 150 bp to 200 bp in length was recovered from the gel and purified. DNA was subjected to PCR using the purified DNA as a template and purified. Absorbance of the resultant DNA was measured to check the concentration and purity thereof

(v) Acquisition of Data by Next Generation Sequencer

Using GAII DNA Sample Cluster Generation Kit (purchased from Illumina) and 36-Cycle SBS Sequencing Kit (purchased from Illumina), a cluster was formed in accordance with the operation manual attached and data were obtained by Genome Analyzer II (Illumina). A sample (3 pmol) derived from a single cell line was used per lane. The number of cycles performed was 36 or 76.

(vi) Data Analysis

Using IPAR/GAPipeline manufactured by Illumina, TXT-form sequence data were prepared from an image of TIFF-form and converted into FASTQ-form. Thereafter, alignment was performed by use of MAQ with reference to Refseq sequence data of a related gene. To the SNPs data extracted, e.g., amino acid substitution data were added and converted into GFF-form by use of built-in software. Mutation information and depth information (mutation or loss of expression information) were checked by use of Gbrowse. The results are shown in Table 6.

TABLE 6
Presence or absence of mutation or loss of expression
of B-Raf and PTEN in human melanoma cell line

| | | | Mutation or Loss | |
|---|---|---|---|---|
| | | Culture | of expression | |
| cell line | Manufacturer | medicum | B-Raf | PTEN |
| SK-MEL-2 | ATCC | MEM | None | None |
| MeWo | ATCC | MEM | None | None |
| CHL-1 | ATCC | DMEM | None | None |
| HMV-1 | Dainippon Pharma | RPMI1640 | None | None |
| | Co, Ltd. | | | |
| HMCB | ATCC | MEM | None | None |
| MDA-MB-435 | Dr. Mary J; C. | RPM1640 | V600E | None |
| | Hendrix at the | | | |
| | University of | | | |
| | Arizona | | | |
| LOX | Dainippon Pharma | RPMI1640 | V600E | None |
| | Co, Ltd. | | | |
| G361 | Dainippon Pharma | MacCoy's | V600E | None |
| | Co, Ltd. | | | |
| FEM | Dr. Fodstad at the | RPMI1640 | A145V | None |
| | Norw. Rad. Hosp. | | | |
| SEKI | Dainippon Pharma | RPMI1640 | V600E | None |
| | Co., Ltd. | | | |
| SK-MEL-28 | ATCC | MEM | V600E | T167A |
| A375 | Dainippon Pharma | RPMI1640 | V600E | Y68H |
| | Co., Ltd. | | | |
| A2058 | ATCC | DMEM | V600E | L112Q |

### Example 2

### Calculation of Anti-Tumor Effect of Angiogenesis Inhibitor on Mouse Model Grafted with Melanoma Cell Line

The human melanoma cell lines used in Example 1 were respectively cultured in the mediums shown in the column of “Culture medium” (containing 10% FBS) of Table 6 until about 80% confluency was obtained (in an incubator under 5% carbon dioxide gas). After culturing, cells were collected by trypsin-EDTA treatment in accordance with a conventional method. The cells were suspended with a phosphate buffer or a matrigel solution (mixture of phosphate buffer and matrigel in a common ratio of 1:1) to prepare suspension solution of 1×10⁸ cells/mL or 5×10⁷ cells/mL. The cell suspension (0.1 mL) was subcutaneously grafted to the side of the body of each nude mouse. In this manner, human melanoma cell line grafted mouse models were prepared.

After grafting, from the time point when a tumor volume reached about 200 mm³, a mesylate of 4-(3-chloro-4-(cyclopropylaminocarbonyl)aminophenoxy)-7-methoxy-6-quinolinecarboxamide (hereinafter referred to as E7080) (100 mg/kg/day) was orally administered. The major axis and minor axis of the tumor were measured everyday by Digimatic Caliper (Mitsutoyo). The tumor volume and specific tumor volume were calculated in accordance with the following formula and thereby the anti-tumor effect (ΔT/C) of an angiogenesis inhibitor on the mouse model was measured.

Tumor volume(TV)=tumor longest diameter(mm)×tumor short diameter²(mm²)/2

Anti-tumor effect(ΔT/C)=(TV of medicinal drug administered group−TV before initiation of administration)/(TV of control group−TV before initiation of administration)×100

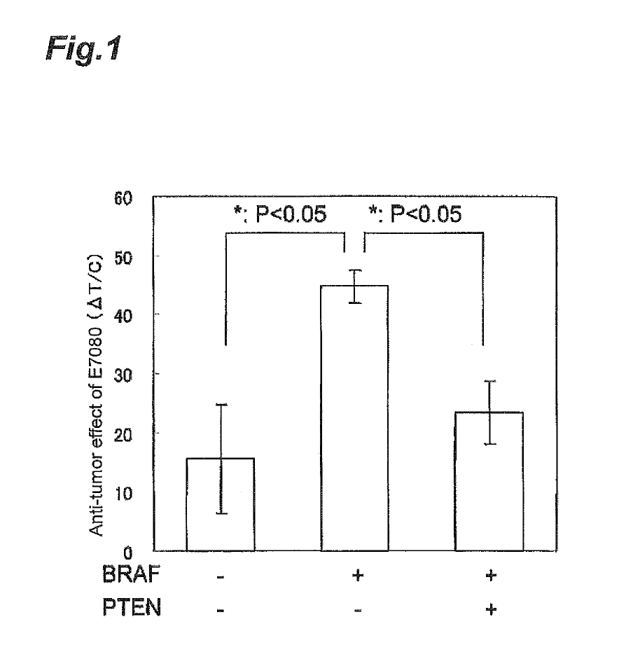

The anti-tumor effect ΔT/C on the 7th day after initiation of administration is shown in FIG. 1. In FIG. 1, the same cell lines used in Example 1 were used.

The anti-tumor effects (ΔT/C) of E7080 on melanoma cells of the case where both of BRAF and PTEN are wild type, the case where both of BRAF and PTEN have a mutation or loss of expression, and the case where BRAF has a mutation and PTEN has no mutation, were 16, 23 and 45%, respectively. The anti-tumor effect of E7080 was observed particularly in the case where both of BRAF and PTEN have a mutation or loss of expression and in the case where BRAF and PTEN do not have a mutation or loss of expression. It is clear that the anti-tumor effect of E7080 varies between melanoma cells which are classified based on the presence or absence of a mutation in BRAF and PTEN (FIG. 1). It was demonstrated that the presence or absence of a mutation in BRAF and PTEN is used as an indicator for predicting the effect of E7080.

### Example 3

### Correlation with the Ratio of Blood Vessels Covered with Periderm Cells (pericytes) Depending Upon the Presence or Absence of a Mutation or Loss of Expression in BRAF and PTEN

As a result of imperfect angiogenesis in a tumor tissue, a phenomenon where blood vessels covered with periderm cells is not formed is observed. In the case where the tumor cells were classified based on the presence or absence of a mutation or loss of expression in BRAF and PTEN, whether the ratio of blood vessels covered with periderm cells changes or not was investigated.

Human melanoma cell line grafted mouse models were prepared using the human melanoma cell lines used in Example 1 in accordance with the method of Example 2. After grafting, at the time point when a tumor volume reached about 100-300 mm³, the mouse was sacrificed with CO₂ and the grafted tumor tissue was excised out by a surgical operation.

Thereafter, from the tumor tissue excised out, a tumor tissue sections were prepared. The sections were stained.

To describe this procedure more specifically, the portion at a distance about 5 mm inside from the periphery of the tumor tissue was cut by knife, the tissue was embedded in OCT Compound. Thereafter, the tissue was frozen with dry ice and a frozen tissue was prepared at −80° C. From the frozen tissue, sections of 8 μm in thickness were prepared, attached to a slide glass, washed with running water and allowed to stand in cold acetone at 4° C. for 10 minutes to prepare samples. Thereafter, the samples were washed three times with a 0.01 M phosphate buffer containing 0.1% Tween20 (hereinafter referred to as washing PBS) and allowed to react with an avidin blocking solution in DAKO biotin blocking kit for 10 minutes at room temperature. After completion of the reaction, the sample was washed three times with washing PBS, and allowed to react with a biotin blocking solution in the kit at room temperature for 10 minutes. Thereafter, the sample was washed three times with washing PBS and allowed to react with normal serum in VECTOR STAIN ABC peroxidase rat IgG kit at room temperature for 20 minutes. After the reaction solution was removed from the samples, a primary antibody, i.e., an anti-CD31 antibody (name of clone: MEC13.3, rat IgG, PharMingen, BD Biosciences), which was diluted 600 times with a 0.1 M phosphate buffer containing a 1% fetal bovine serum, was added and allowed to react overnight at 4° C. Thereafter, the samples were washed, and a secondary antibody labeled with biotin in the kit was added and allowed to react at room temperature for 30 minutes. The samples were washed and then further reacted with an avidin reagent in the kit at room temperature for 30 minutes. Thereafter, the samples were washed three times with a 0.01 M phosphate buffer and color was developed with DAB to stain CD31.

Subsequently, the samples were washed with running water and washed three times with Tris buffer. Thereafter, with the samples, alkali phosphatase-labeled anti-α-SMA antibody (name of clone: 1A4, mouse IgG, SIGMA-ALDRICH) diluted 100 times with Tris buffer was allowed to react at room temperature for one hour. Thereafter, the samples were washed three times with Tris buffer and color was developed with a fuchsine solution in DAKO LSAB kit to stain α-SMA.

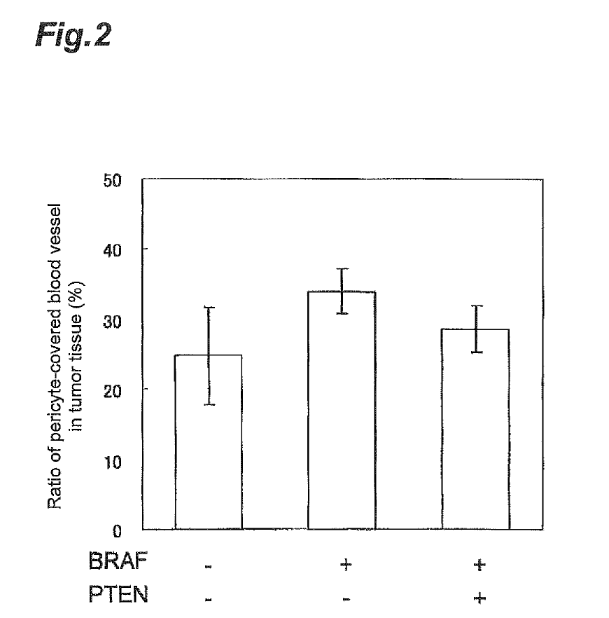

Each of the samples stained was placed under a microscope and the number of blood vessels and the number of blood vessels covered with pericytes were counted by a CCD camera HYPER SCOPE (KEYENCE) in e.g., 5 sites per sample, and averaged. The number of blood vessels or pericytes per unit area was obtained. Furthermore, the ratio of the blood vessels with pericytes in the number of the blood vessels was calculated in each melanoma cell line. The results are shown in FIG. 2. In FIG. 2, the same cell lines used in Example 1 were used.

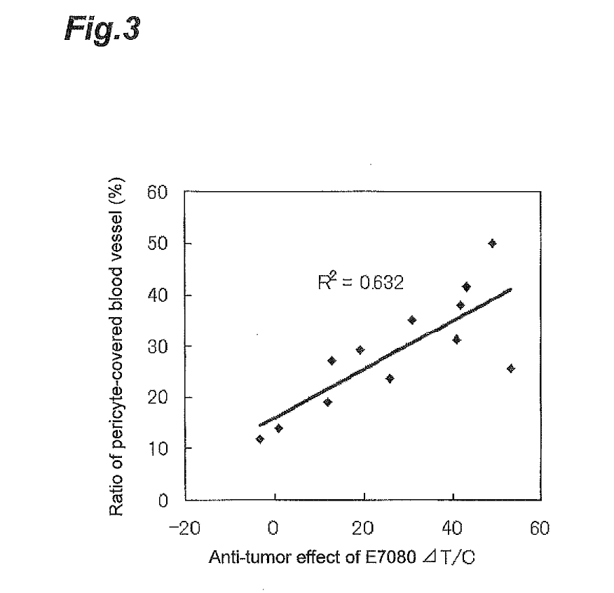

Between the classes of the melanoma cells, which were classified based on the presence or absence of a mutation or loss of expression in BRAF and PTEN, the ratio of the blood vessels covered with pericytes in the tumor tissue tended to differ (FIG. 2). Furthermore, the anti-tumor effect of E7080 and the ratio of the blood vessels covered with pericytes in a tumor tissue were analyzed. As a result, the ratio of the blood vessels covered with pericytes in a tumor tissue exhibited high correlation with the anti-tumor effect of E7080 (FIG. 3). From this, it was suggested that the presence or absence of a mutation or loss of expression in BRAF and PTEN influences the properties of blood vessels in a tumor and varies the ratio of blood vessels covered with pericytes in the tumor. The results suggest that, in melanoma where the ratio of blood vessels covered with pericytes is low, E7080 may possibly tend to have an effect on the blood vessels, and that particularly in cases where both of BRAF and PTEN are wild type and where both of BRAF and PTEN have a mutation or loss of expression, E7080 easily produces an effect.

### Example 4

### Expression Regulation of ANG1 and ANG2 by the Presence or Absence of a Mutation or Loss of Expression in BRAF and PTEN

(1) Correlation Between the Presence or Absence of a Mutation or Loss of Expression in BRAF and PTEN and the Expressions of ANG1 and ANG2

In human melanoma cell lines, the correlation between the presence or absence of a mutation or loss of expression in BRAF and PTEN and the expressions of ANG1 and ANG2 was investigated by using the quantitative RT-PCR and ELISA.

1. Investigation by Quantitative RT-PCR Method

From each of the human melanoma cell lines used in Example 1, total RNA was prepared in the same method as in Example 1 (2) (i) and subjected to the following measurements.

The quantitative RT-PCR method for various types of angiogenesis factors and various types of angiogenesis factor receptors was performed by use of a gene specific probe (TaqMan Gene Expression Assays mixture, purchased through ASSAYS-ON-DEMAND of Applied Biosystems) and gene analysis BioMark™ system (purchased from Fluidigm) based on the operation manual, as follows.

The names of genes of the angiogenesis factors and angiogenesis factor receptors used herein and the assay ID of the probes purchased are shown in Table 7.

The operation consisting of three stages, a reverse transcription reaction, a pre-amplification and PCR, was performed.

In the first stage, i.e., reverse transcription reaction, RNase Free dH2O (6.5 μL) was added to the RNA prepared. To this, 5× PrimeScript buffer (2 μL), PrimeScript RT Enzyme Mix I (0.5 μL), Oligo dT Primer (50 μM) (0.5 μL) and Random 6 mer (100 μM) (0.5 μL) were further added. After allowed to react at 37° C. for 15 minutes, the mixture was heated at 85° C. for 5 seconds to terminate the reaction to obtain a cDNA solution. The obtained cDNA solution was subjected to the second stage, pre-amplification reaction.

In the pre-amplification reaction, a low-TE buffer (58 μL) was added to gene specific Primer/Probe (42 μL). From the solution, an aliquot (56.25 μL) was taken. To this, PreAmp Master Mix (112.5 μL) and the cDNA solution (56.25 μL) were added. The mixture was allowed to react at 95° C. for 10 minutes, and then, a reaction cycle consisting of a reaction at 95° C. for 15 seconds and a reaction at 60° C. for 4 minutes was repeated 14 times. After completion of the reaction, the reaction solution was diluted (1:5) with TE buffer, and used as a pre-amplification solution. The obtained pre-amplification solution was subjected to the third stage, PCR.

A sample solution was prepared by adding Loading Buffer (27.5 μL) to 2×ABI Master Mix (275 μL) and further adding, to 5.5 μL of the solution, 4.5 μL of the pre-amplification solution. Furthermore, an assay solution was prepared by adding 10% Tween to water (4750 μL), taking an aliquot (5 μL) from the solution, and adding 20× assays (5 μL) to the aliquot. The sample solution and the assay solution were separately added to 48.48 Dynamic tray and a sample was loaded by NanoFlex ICF controller and thereafter, measurement was performed by Biomark (Fluidigm Corporation).

TABLE 7
Angiogenesis factor and angiogenesis factor receptor quantified

| No. | Gene Name | Assay ID |
|---|---|---|
| 1 | beta-actin | Hs99999903_m1 |
| 2 | Ang-1 | Hs 00181613 |
| 3 | Ang-2 | Hs 00169867 |

For performing quantitative analysis of each gene from the obtained PCR product, a calibration curve was established by use of a mRNA sample prepared by adding equivalent amounts of all samples. The expression level of a gene in each of the melanoma cell lines was obtained by calculating Ct (stands for a threshold cycle value which is the number of cycles of PCR required for a PCR product to reach a predetermined concentration) from the calibration curve. The expression level of a gene in each melanoma cell line was corrected by β-actin expression level to obtain an expression level ratio of the gene in the melanoma cell line and used for comparison analysis.

2. Validation by ELISA

Human melanoma cell line grafted mouse models were prepared in accordance with the method of Example 2 by using the human melanoma cells used in Example 1. After grafting, in the stage where a tumor volume reached 100 mm³ or more, the mouse was sacrificed and the tumor tissue grafted was recovered. To the tumor tissue, a cell lysis buffer (purchased from Cell Signaling) was added to prepare a cell sap and stored at −80° C. The expression level of ANG1 protein and the expression level of ANG2 protein in the preparation solution and the ratio of them (ANG1/ANG2) were determined by an ELISA Kit (purchased from R&D systems) and quantified based on a calibration curve.

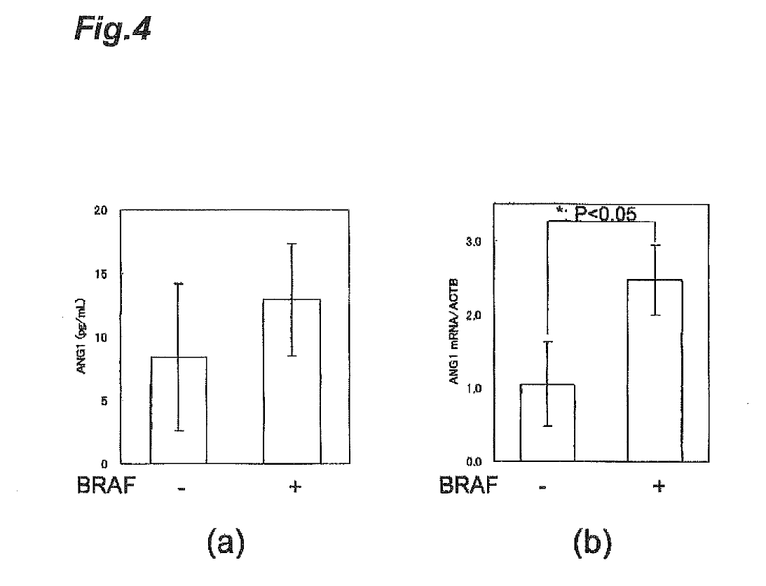

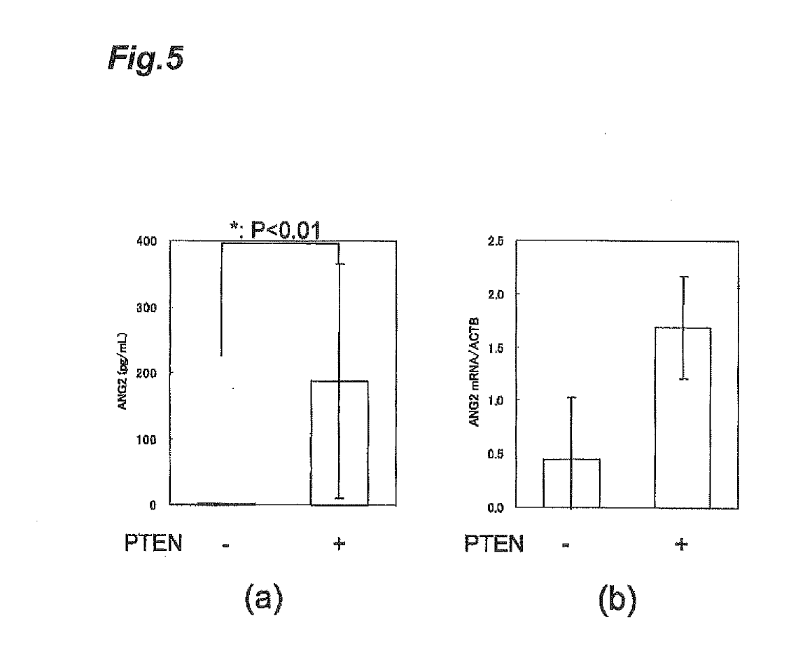

The results are shown in FIGS. 4 and 5. In FIG. 4, the bar indicated by BRAF “−” includes the results of anti-tumor effects of SK-MEL-2, MeWo, CHL-1, HMV-1 and HMCB cell lines shown in Table 6; whereas, the bar of BRAF “+” includes the results of anti-tumor effects of MDA-MB-435, LOX, G361, FEM, SEKI, SK-MEL-28, A375 and A2058 cell lines shown in Table 6. In FIG. 5, the bar indicated by PTEN “−” includes the results of anti-tumor effects of SK-MEL-2, MeWo, CHL-1, HMV-1, HMCB, MDA-MB-435, LOX, G361, FEM and SEKI cell lines shown in Table 6; whereas a bar indicated by PTEN “+” includes the results of anti-tumor effects of SK-MEL-28, A375 and A2058 cell lines shown in Table 6.

It was demonstrated that the mRNA expression level of ANG1 (No. 11 in Table 7) significantly increases in a human melanoma cell line in which BRAF has a mutation (FIG. 4 (b)) and the expression level of ANG1 protein increases in a human melanoma cell line in which BRAF has a mutation (FIG. 4 (a)).

Furthermore, the mRNA expression level of ANG2 (No. 12 in Table 7) increases in a human melanoma cell line in which PTEN has a mutation (FIG. 5 (b)), and the expression level of ANG2 protein (No. 12 in Table 7) significantly increases in a human melanoma cell line in which PTEN has a mutation (FIG. 5 (a)).

(2) Correlation Between the Presence or Absence of a Mutation or Loss of Expression in BRAF and PTEN and the Ratio (ANG1/ANG2) of Expression Levels of ANG1 and ANG2

It is known that ANG1 binds to TIE-2 receptor and the ANG1-TIE-2 signal induces maturation of blood vessels. To TIE-2 receptor, ANG2 and ANG1 competitively bind. If ANG2 binds to the receptor, no signal flows downstream. This state becomes equivalent to the state where ANG1 is not expressed (or expression is low). In other words, if BRAF is normal, it is known that a signal of VEGF contributes to angiogenesis and survival, and maturation of blood vessels does not occur

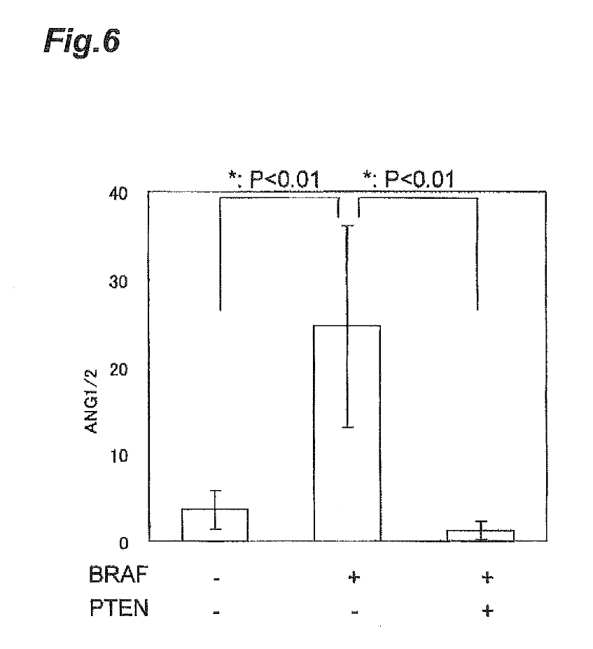

When melanoma cells are classified based on the presence or absence of a mutation of BRAF and PTEN, whether the ANG1-11E-2 signaling is influenced was investigated based on the ratio (ANG1/ANG2) of the expression level of ANG1 protein and the expression level of ANG2 protein. The results are shown in FIG. 6. In FIG. 6, the cell lines used in Example 1 were used.

When melanoma cells are classified based on the presence or absence of a mutation of BRAF and PTEN, the ratio (ANG1/ANG2) of the expression level of ANG1 protein and the expression level of ANG2 protein tended to be low in the case where both of BRAF and PTEN are wild type and hi the case where BRAF and PTEN have a mutation or a loss of expression; whereas, the ratio (ANG1/ANG2) tended to be high in the case where BRAF has a mutation or loss of expression and PTEN is wild type (FIG. 6). Furthermore, compared to the case where BRAF has a mutation or a loss of expression and PTEN is wild type, the ratio of expression levels of ANG1 and ANG2 was shown to be significantly low in the case where both of BRAF and PTEN are wild type and in the case where BRAF and PTEN have a mutation or loss of expression. This means that in a mutation or loss of expression of BRAF and PTEN, a combination of mutations likely responsive to E7080 and ANG1/ANG2 exhibit similar tendencies.

From the above results, it was elucidated that the anti-tumor effect of an angiogenesis inhibitor is defined by the presence or absence of a mutation or loss of expression in BRAF and PTEN. In a subject having no BRAF mutation, the ratio of periderm cells around a blood vessel in a tumor tissue decreases. It was elucidated that expression control of ANG1 and ANG2, which are involved in formation of pericyte-covered blood vessels, is involved in the cause thereof. In the case where ANG1 is not expressed or the expression level of ANG1 is low, in other words, in the state where BRAF is normal, particularly if FGFR3 is expressed, an FGFR kinase inhibitor is expected to directly kill cancer cells. This suggested that E7080 has a potential to not only inhibit angiogenesis but also enhance an anti-tumor effect in a melanoma patient in which BRAF is normal and FGFR3 is expressed.

In contrast, it was suggested that if ANG1 is highly expressed, in other words, in the case where BRAF has a mutation or loss of expression, maturation of blood vessels varies depending upon the ANG2 expression; whereas if ANG2 is highly expressed, in other words, in the case where PTEN has a mutation or loss of expression, no maturation occurs because of competitive inhibition by ANG1 and ANG2; conversely, if the expression of ANG2 is low, in other words, in the case where PTEN is normal, maturation of blood vessels occurs. Therefore, it was suggested that, in the case where ANG1 and ANG2 expressions are high, the anti-tumor effect of E7080 due to angiogenesis inhibition can be expected; whereas, in the case where ANG1 expression is high and ANG2 expression is low and if ANG2 expression is higher than ANG1 expression, the anti-tumor effect of E7080 can be expected.

Accordingly, it became possible that the effect of E7080 can be predicted based on ANG1 and ANG2 expression levels or the ratio of the expression levels.

### Example 5

### Correlation Between the Presence or Absence of a Mutation or Loss of Expression in BRAF and PTEN and the Expressions of SHC1, IL6, CXCR4, COL4A3, NRP2, MEIS1, ARHGAP22, SCG2, FGF9, PML, FGFR1, FGFR4 and VEGFR1

In human melanoma cell lines, the correlation between the presence or absence of a mutation or loss of expression in BRAF and PTEN and the expressions of SHC1, IL6, CXCR4, COL4A3, NRP2, MEIS1, ARHGAP22, SCG2, FGF9, PML, FGFR1, FGFR4 and VEGFR1 was investigated by use of a DNA microarray method.

1. Extraction of Total RNA from Sample

Human melanoma cells used in Example 1 were used to prepare human melanoma cell grafted mouse models in the same manner as in Example 2. After grafting, in the stage where a tumor volume reached 200 mm³ or more, the mouse was sacrificed and the tumor tissue grafted was excised out. Total. RNA was prepared in the same method as in Example 1 (2) (i) and subjected to the following operations.

2. RNA Quantification

1) Quantification by DNA Microarray

cDNA synthesis and biotin labeling were performed based on the method of Schena et al. (Schena et al., Science, 1995, 270, p. 467-470), the method of Lockhart et al. (Lockhart et al., Nature Biotechnology, 1996, 14, p. 1675-1680) or the latest operation manual of GeneChip (registered trade mark) Array Station manufactured by Affimetrix. Thereafter, hybridization with a DNA microarray (Human Genome U133 Plus 2.0 Array) manufactured by Affimetrix and measurement were performed based on the operation manual to obtain data.

3. Data Analysis

Data were statistically analyzed by a trend test using the cumulative chi-square method and genes which showed a significant change in expression level by the presence or absence of a mutation or loss of expression in BRAF and PTEN were extracted.

As a result, it was elucidated that the expression levels of IL6, CXCR4, COL4A3, MEIS1, FGF9, FGFR1, FGFR4 and VEGFR1 significantly decrease from the expression level of a case where BRAF has a mutation and PTEN is wild type; whereas, the expression levels of SHC1, NRP2, ARHGAP22, SCG2 and PML significantly increase from the expression level of a case where BRAF has a mutation and PTEN is wild type. Thus, it was elucidated that the expression levels of SHC1, IL6, CXCR4, COL4A3, NRP2, MEIS1, ARHGAP22, SCG2, FGF9, PML, FGFR1, FGFR4 and VEGFR1 significantly change when melanoma cells were classified based on the presence or absence of a mutation or loss of expression in BRAF and PTEN.

More specifically, if the responsiveness of a subject to an angiogenesis inhibitor is high, it was suggested that

the expression level of SHC1 significantly decreases compared to a control value,

the expression level of IL6 significantly increases compared to a control value,

the expression level of CXCR4 significantly increases compared to a control value,

the expression level of COL4A3 significantly increases compared to a control value,

the expression level of NRP2 significantly decreases compared to a control value,

the expression level of MEIS 1 significantly increases compared to a control value,

the expression level of ARHGAP22 significantly decreases compared to a control value,

the expression level of SCG2 significantly decreases compared to a control value,

the expression level of FGF9 significantly increases compared to a control value,

the expression level of PML significantly decreases compared to a control value,

the expression level of FGFR1 significantly increases compared to a control value,

the expression level of FGFR4 significantly increases compared to a control value, and/or

the expression level of VEGFR1 significantly increases compared to a control value.

### Example 6

### Correlation Between the Presence or Absence of a Mutation or Loss of Expression in BRAF and PTEN and Expressions of FGFR3 and FGFR2

In human melanoma cell lines, the correlation between the presence or absence of a mutation or loss of expression in BRAF and PTEN and the expressions of FGFR3 and FGFR2 was investigated by a quantitative RT-PCR method.

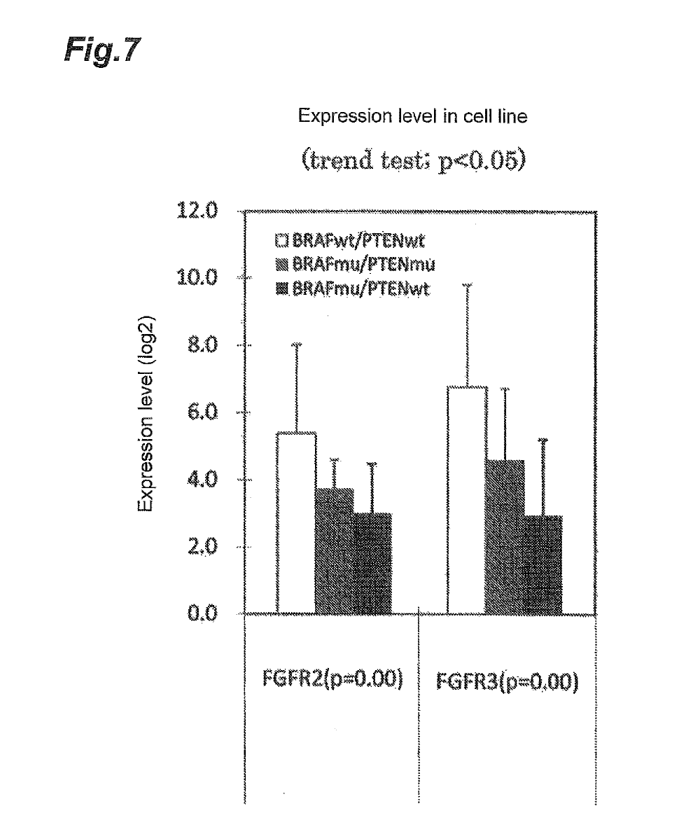

Human melanoma cell lines used in Example 1 were used to prepare human melanoma cell grafted mouse models in the same manner as in Example 2. Total RNA was prepared in the same method as in Example 1 (2) (i) and a quantitative RT-PCR was performed in the same method as in Example 4, Section 1. The name of genes of the angiogenesis factors and angiogenesis factor receptors used herein and the assay ID of the probe purchased are shown in Table 8 and the results are shown in FIG. 7.

TABLE 8
Angiogenesis factor and angiogenesis factor receptor quantified

| No. | Gene Name | Assay ID |
|---|---|---|
| 1 | beta-actin | Hs99999903_m1 |
| 2 | FGFR3 | Hs_00179829 |
| 3 | FGFR2 | Hs_00256527 |

As a result, the expression levels of FGFR3 and FGFR2 significantly decrease from the expression level of a case where BRAF has a mutation and PTEN is wild type. It was thus elucidated that the expression levels of FGFR3 and FGFR2 significantly change when melanoma cells were classified based on the presence or absence of a mutation or loss of expression in BRAF and PTEN (FIG. 7).

More specifically, it was suggested that if the responsiveness of a subject to an angiogenesis inhibitor is high,

the expression level of FGFR2 significantly increases compared to a control value, and

the expression level of FGFR3 significantly increases compared to a control value.

### INDUSTRIAL APPLICABILITY

The present invention provides a method for predicting the responsiveness of a subject to an angiogenesis inhibitor. The prediction results obtained by the method of the present invention can be used as information for selecting an angiogenesis inhibitor for treating a tumor.

### Sequence Listing Free Text

SEQ ID NO: 1: B-Raf Polynucleotide Sequence, GenBank Accession No. NM_{—}004333.4

SEQ ID NO: 2: B-Raf Amino Acid Sequence, GenBank Accession No. NP_{—}004324.2

SEQ ID NO: 3: PTEN Polynucleotide Sequence, GenBank Accession No. NM_{—}000314.4

SEQ ID NO: 4: PTEN Amino Acid Sequence, GenBank Accession No. NP_{—}000305.3

SEQ ID NO: 5: SHC1 Polynucleotide Sequence, GenBank Accession No. NM_{—}003029.4

SEQ ID NO: 6: SHC1 Amino Acid Sequence, GenBank Accession No. NP_{—}003020.2

SEQ ID NO: 7: IL6 Polynucleotide Sequence, GenBank Accession No. NM_{—}000600.3

SEQ ID NO: 8: IL6 Amino Acid Sequence, GenBank Accession No. NP_{—}000591.1

SEQ ID NO: 9: CXCR4 Polynucleotide Sequence, GenBank Accession No. NM_{—}001008540.1

SEQ ID NO: 10: CXCR4 Amino Acid Sequence, GenBank Accession No. NP_{—}001008540.1

SEQ ID NO: 11: COL4A3 Polynucleotide Sequence, GenBank Accession No. NM_{—}000091.4

SEQ ID NO: 12: COL4A3 Amino Acid Sequence, GenBank Accession No. NP_{—}000082.2

SEQ ID NO: 13: NRP2 Polynucleotide Sequence, GenBank Accession No. NM_{—}003872.2

SEQ ID NO: 14: NRP2 Amino Sequence, GenBank Accession No. NP_{—}003863.2

SEQ ID NO: 15: MEIS1 Polynucleotide Sequence, GenBank Accession No. NM_{—}002398.2

SEQ ID NO: 16: MEIS1 Amino Acid Sequence, GenBank Accession No. NP_{—}002389.1

SEQ ID NO: 17: ARHGAP22 Polynucleotide Sequence, GenBank Accession No. NM_{—}021226.2

SEQ ID NO: 18: ARHGAP22 Amino Acid Sequence, GenBank Accession No. NP_{—}067049.2

SEQ ID NO: 19-44: Synthetic DNA

SEQ ID NO: 45: ANG1 Polynucleotide Sequence, GenBank Accession No. NM_{—}001146.3

SEQ ID NO: 46: ANG1 Amino Acid Sequence, GenBank Accession No. NP_{—}001137.2

SEQ ID NO: 47: ANG2 Polynucleotide Sequence, GenBank Accession No. NM_{—}001118888.1

SEQ ID NO: 48: ANG2 Amino Acid Sequence, GenBank Accession No. NP_{—}001112360.1

SEQ ID NO: 49: SCG2 Polynucleotide Sequence, GenBank Accession No. NM_{—}003469.4

SEQ ID NO: 50: SCG2 Amino Acid Sequence, GenBank Accession No. NP_{—}003460.2

SEQ ID NO: 51: FGF9 Polynucleotide Sequence, GenBank Accession No. NM_{—}002010.2

SEQ ID NO: 52: FGF9 Amino Acid Sequence, GenBank Accession No. NP_{—}002001.1

SEQ ID NO: 53: PML Polynucleotide Sequence, GenBank Accession No. NM_{—}002675.3

SEQ ID NO: 54: PML Amino Acid Sequence, GenBank Accession No. NP_{—}002666.1

SEQ ID NO: 55: FGFR3 Polynucleotide Sequence, GenBank Accession No. NM_{—}000142.3

SEQ ID NO: 56: FGFR3 Amino Acid Sequence, GenBank Accession No. NP_{—}000133.1

SEQ ID NO: 57: FGFR2 Polynucleotide Sequence, GenBank Accession No. 7. NM_{—}001144918.1

SEQ ID NO: 58: FGFR2 Amino Acid Sequence, GenBank Accession No. NP_{—}001138390.1

SEQ ID NO: 59: FGFR1 Polynucleotide Sequence, GenBank Accession No. NM_{—}001174063.1

SEQ ID NO: 60: FGFR1 Amino Acid Sequence, GenBank Accession No. NP_{—}001167534.1

SEQ ID NO: 61: FGFR4 Polynucleotide Sequence, GenBank Accession No. NM_{—}002011.3

SEQ ID NO: 62: FGFR4 Amino Acid Sequence, GenBank Accession No. NP_{—}002002.3

SEQ ID NO: 63: VEGFR1 Polynucleotide Sequence, GenBank Accession No. NM_{—}001159920.1

SEQ ID NO: 64: VEGFR1 Amino Acid Sequence, GenBank Accession No. NP_{—}001153392.1

1. A method for predicting the responsiveness of a subject suffering from a tumor to an angiogenesis inhibitor, comprising
(a) detecting the presence or absence of a mutation or loss of expression of B-Raf and the presence or absence of a mutation or loss of expression of PTEN in a sample derived from a tumor tissue of the subject, wherein in the detection step, a case where
(a1) B-Raf is wild type and PTEN is wild type, or
(a2) B-Raf has at least one mutation selected from Table 1 or loss of expression and PTEN has at least one mutation selected from Table 2 or loss of expression
is indicative of the high responsiveness of the subject to the angiogenesis inhibitor, wherein the angiogenesis inhibitor is 4-(3-chloro-4-(cyclopropylaminocarbonyl)aminophenoxy)-7-methoxy-6-quinolinecarboxamide or a pharmacologically acceptable salt thereof.

2. The method according to claim 1, wherein, in the detection step (a), a case where B-Raf is wild type and PTEN is wild type is indicative of the high responsiveness of the subject to the angiogenesis inhibitor.

3. The method according to claim 1, wherein, in the detection step (a), a case where B-Raf has at least one mutation selected from Table 1 or loss of expression and PTEN has at least one mutation selected from Table 2 or loss of expression is indicative of the high responsiveness of the subject to the angiogenesis inhibitor.

4. The method according to claim 1, wherein the mutation of B-Raf is a V600E mutation in an amino acid sequence or a mutation in a nucleotide sequence corresponding to the mutation.

5. The method according to claim 1, wherein the mutation of PTEN is at least one mutation in a nucleotide sequence selected from the group consisting of A499G, T202C and T335A or at least one mutation in an amino acid sequence selected from the group consisting of T167A, Y68H and L112Q.

6. (canceled)

7. The method according to claim 1, wherein the angiogenesis inhibitor is a mesylate salt of 4-(3-chloro-4-(cyclopropylaminocarbonyl)aminophenoxy)-7-methoxy-6-quinolinecarboxamide.

8. The method according to claim 1, wherein the tumor is a tumor having a V600E mutation in B-Raf.

9. The method according to claim 1, wherein the tumor is melanoma, thyroid cancer, colorectal cancer, ovarian cancer, liver cancer, lung cancer, endometrial cancer or glioma.

10. The method according to claim 1, wherein, in the step (a), the high responsiveness of the subject to the angiogenesis inhibitor is predicted; and the method further comprises a step (b) of quantifying expression levels of ANG1 and ANG2 in the sample derived from the tumor tissue of the subject, wherein, in the quantification step, a case where
(b1) the expression level of ANG1 is low compared to a control value
(b2) the expression level of ANG2 is high compared to a control value, or
(b3) the ratio of the expression levels of ANG1 and ANG2 is low compared to a control value
is indicative of the high responsiveness of the subject to the angiogenesis inhibitor.

11. The method according to claim 1, wherein, in the step (a), the high responsiveness of the subject to the angiogenesis inhibitor is predicted; and the method further comprises a step (c) of quantifying an expression level of at least one selected from the group consisting of SHC1, IL6, CXCR4, COL4A3, NRP2, MEIS1, ARHGAP22, SCG2, FGF9, PML, FGFR3, FGFR2, FGFR1, FGFR4 and VEGFR1 in the sample derived from the tumor tissue of the subject, wherein, in the quantification step, a case where
(c1) the expression level of SHC1 is low compared to a control value,
(c2) the expression level of NRP2 is low compared to a control value,
(c3) the expression level of ARHGAP22 is low compared to a control value,
(c4) the expression level of SCG2 is low compared to a control value,
(c5) the expression level of PML is low compared to a control value,
(c6) the expression level of IL6 is high compared to a control value,
(c7) the expression level of CXCR4 is high compared to a control value,
(c8) the expression level of COL4A3 is high compared to a control value,
(c9) the expression level of MEIS1 is high compared to a control value,
(c10) the expression level of FGF9 is high compared to a control value,
(c11) the expression level of FGFR3 is high compared to a control value,
(c12) the expression level of FGFR2 is high compared to a control value,
(c13) the expression level of FGFR1 is high compared to a control value,
(c14) the expression level of FGFR4 is high compared to a control value, or
(c15) the expression level of VEGFR1 is high compared to a control value is indicative of the high responsiveness of the subject to the angiogenesis inhibitor.

12. A method for predicting the responsiveness of a subject suffering from a tumor to an angiogenesis inhibitor, comprising
(b) quantifying expression levels of ANG1 and ANG2 in a sample derived from a tumor tissue of the subject, wherein, in the quantification step, a case where
(b1) the expression level of ANG1 is low compared to a control value
(b2) the expression level of ANG2 is high compared to a control value, or
(b3) the ratio of expression level of ANG1 and ANG2 is low compared to a control value
is indicative of the high responsiveness of the subject to the angiogenesis inhibitor, wherein the angiogenesis inhibitor is 4-(3-chloro-4-(cyclopropylaminocarbonyl)aminophenoxy)-7-methoxy-6-quinolinecarboxamide or a pharmacologically acceptable salt thereof.

13. The method according to claim 12, wherein, in the step (b), high responsiveness of the subject to the angiogenesis inhibitor is predicted, and the method further comprises a step (c) of quantifying an expression level of at least one selected from the group consisting of SHC1, IL6, CXCR4, COL4A3, NRP2, MEIS1, ARHGAP22, SCG2, FGF9, PML, FGFR3, FGFR2, FGFR1, FGFR4 and VEGFR1 in the sample derived from the tumor tissue of the subject, wherein, in the quantification step, a case where
(c1) the expression level of SHC1 is low compared to a control value,
(c2) the expression level of NRP2 is low compared to a control value,
(c3) the expression level of ARHGAP22 is low compared to a control value,
(c4) the expression level of SCG2 is low compared to a control value,
(c5) the expression level of PML is low compared to a control value,
(c6) the expression level of IL6 is high compared to a control value,
(c7) the expression level of CXCR4 is high compared to a control value,
(c8) the expression level of COL4A3 is high compared to a control value,
(c9) the expression level of MEIS1 is high compared to a control value,
(c10) the expression level of FGF9 is high compared to a control value,
(c11) the expression level of FGFR3 is high compared to a control value,
(c12) the expression level of FGFR2 is high compared to a control value,
(c13) the expression level of FGFR1 is high compared to a control value,
(c14) the expression level of FGFR4 is high compared to a control value, or
(c15) the expression level of VEGFR1 is high compared to a control value is indicative of the high responsiveness of the subject to the angiogenesis inhibitor.

14. A method for treating a subject suffering from a tumor by administration of an angiogenesis inhibitor, wherein the subject has been predicted to be highly responsive to the angiogenesis inhibitor by the method according to claim 1, wherein the angiogenesis inhibitor is 4-(3-chloro-4-(cyclopropylaminocarbonyl)aminophenoxy)-7-methoxy-6-quinolinecarboxamide or a pharmacologically acceptable salt thereof.

15. (canceled)

16. The method according to claim 14, wherein the angiogenesis inhibitor is a mesylate salt of 4-(3-chloro-4-(cyclopropylaminocarbonyl)aminophenoxy)-7-methoxy-6-quinolinecarboxamide.

17-19. (canceled)

20. A pharmaceutical composition comprising an angiogenesis inhibitor for treating a subject suffering from a tumor, wherein the subject has been predicted to be highly responsive to the angiogenesis inhibitor by the method according to claim 1, and wherein the angiogenesis inhibitor is 4-(3-chloro-4-(cyclopropylaminocarbonyl)aminophenoxy)-7-methoxy-6-quinolinecarboxamide or a pharmacologically acceptable salt thereof.

21. (canceled)

22. The pharmaceutical composition according to claim 20, wherein the angiogenesis inhibitor is a mesylate salt of 4-(3-chloro-4-(cyclopropylaminocarbonyl)aminophenoxy)-7-methoxy-6-quinolinecarboxamide.

23. A kit for predicting the responsiveness of a subject suffering from a tumor to an angiogenesis inhibitor, comprising probes of B-Raf and PTEN or probes of ANG1 and ANG2, wherein the responsiveness of the subject suffering from the tumor to the angiogenesis inhibitor is predicted by the method according to claim 1, and wherein the angiogenesis inhibitor is 4-(3-chloro-4-(cyclopropylaminocarbonyl)aminophenoxy)-7-methoxy-6-quinolinecarboxamide or a pharmacologically acceptable salt thereof.

24. A method for treating a subject suffering from a tumor by administration of an angiogenesis inhibitor, wherein the subject has been predicted to be highly responsive to the angiogenesis inhibitor by the method according to claim 12, wherein the angiogenesis inhibitor is 4-(3-chloro-4-(cyclopropylaminocarbonyl)aminophenoxy)-7-methoxy-6-quinolinecarboxamide or a pharmacologically acceptable salt thereof.

25. A pharmaceutical composition comprising an angiogenesis inhibitor for treating a subject suffering from a tumor, wherein the subject has been predicted to be highly responsive to the angiogenesis inhibitor by the method according to claim 12, and wherein the angiogenesis inhibitor is 4-(3-chloro-4-(cyclopropylaminocarbonyl)aminophenoxy)-7-methoxy-6-quinolinecarboxamide or a pharmacologically acceptable salt thereof.

26. A kit for predicting the responsiveness of a subject suffering from a tumor to an angiogenesis inhibitor, comprising probes of B-Raf and PTEN or probes of ANG1 and ANG2, wherein the responsiveness of the subject suffering from the tumor to the angiogenesis inhibitor is predicted by the method according to claim 12, and wherein the angiogenesis inhibitor is 4-(3-chloro-4-(cyclopropylaminocarbonyl)aminophenoxy)-7-methoxy-6-quinolinecarboxamide or a pharmacologically acceptable salt thereof.
